(19) United States
(12) Patent Application Publication
(10) Pub. No.: US 2022/0362165 A1
(43) Pub. Date: Nov. 17, 2022

(54) TETRABENAZINE TRANSDERMAL DELIVERY DEVICE

(71) Applicants: Krunal RAVAL, Ahmedabad, Gujarat (IN); Suresh BORSADIA, Plainsboro, NJ (US); Hock S. TAN, East Brunswick, NJ (US); Kalpana PATEL, Est Windsor, NJ (US); SHINKEI THERAPEUTICS LLC, East Princeton, NJ (US)

(72) Inventors: Krunal RAVAL, Ahmedabad, Gujarat (IN); Suresh BORSADIA, Plainsboro, NJ (US); Hock S. TAN, East Brunswick, NJ (US); Kalpana PATEL, Est Windsor, NJ (US)

(21) Appl. No.: 17/770,857

(22) PCT Filed: Oct. 21, 2020
(86) PCT No.: PCT/US2020/056559

§ 371 Date: Apr. 21, 2022

Related U.S. Application Data
(60) Provisional application No. 62/924,325, filed on Oct. 22, 2019.

(51) Int. Cl. A61K 9/70; A61K 31/4745

(52) U.S. Cl. CPC A61K 9/7061; A61K 31/4745

(57) ABSTRACT

Provided herein are compositions, delivery devices, and methods relating to the delivery of tetrabenazine and/or deuterated tetrabenazine, for example, continuous or substantially continuous delivery such as transdermal delivery, for the treatment of a hyperkinetic movement disorder. Also provided are transdermal delivery devices comprising tetrabenazine, a deuterated tetrabenazine, or a combination thereof, pharmaceutical compositions comprising tetrabenazine and/or a deuterated tetrabenazine, methods of preparing the same, and methods of using the same for example, for the treatment of a hyperkinetic movement disorder.

### CROSS REFERENCE TO RELATED APPLICATIONS

This application claims the benefit of U.S. Provisional Application No. 62/924,325, filed on Oct. 22, 2019, the content of which is incorporated herein by reference in its entirety.

### BACKGROUND OF THE INVENTION

### Field of the Invention

In various embodiments, the present invention generally relates to delivery of tetrabenazine and/or a deuterated tetrabenazine, for example, continuous or substantially continuous delivery, such as transdermal delivery. In various embodiments, the present invention also relates to pharmaceutical compositions and delivery devices, such as transdermal delivery devices comprising tetrabenazine and/or a deuterated tetrabenazine, pharmaceutical compositions comprising tetrabenazine and/or a deuterated tetrabenazine, methods of preparing the same, and methods of using the same.

### Background Art

Tetrabenazine is a vesicular monoamine transporter 2 (VMAT2) inhibitor. Tetrabenazine was approved by the U.S. Food and Drug Administration (FDA) as Xenazine® tablet for oral use, indicated for the treatment of chorea associated with Huntington's disease. The active ingredient in Xenazine® tablet is a racemic mixture of (3R,11bR)-1,3,4,6,7,11b-Hexahydro-9,10-dimethoxy-3-(2-methylpropyl)-2H-benzo[a]quinolizin-2-one (hereinafter “R,R-tetrabenazine or R,R-TBZ”) and (3S,11bS)-1,3,4,6,7,11b-hexahydro-9,10-dimethoxy-3-(2-methylpropyl)-2H-benzo[a]quinolizin-2-one (hereinafter “S,S-tetrabenazine or S,S-TBZ”).

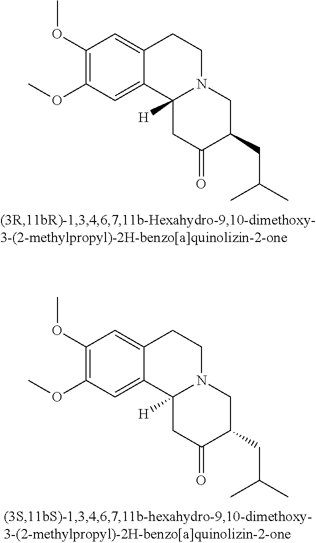

In 2017, the FDA approved a deuterated tetrabenazine, deutetrabenazine, as Austedo™ tablets for oral use, which is also indicated for the treatment of chorea associated with Huntington's disease. The active ingredient in Austedo™ tablet is a selectively deuterium-substituted, stable, non-radioactive isotopic form of tetrabenazine in which the six hydrogen atoms on the two O-linked methyl groups have been replaced with deuterium atoms (i.e. —OCD₃ rather than —OCH₃ moieties). The active ingredient in Austedo™ tablet is also a racemic mixture.

### BRIEF SUMMARY OF THE INVENTION

In various embodiments, the present invention is directed to pharmaceutical compositions, drug delivery devices, methods of preparation, and methods of use such as those relate to delivery of tetrabenazine and/or a deuterated tetrabenazine to a subject bypassing first-pass metabolism, for example, in a continuous or substantially continuous fashion. Without wishing to be bound by theories, the delivery of tetrabenazine and/or a deuterated tetrabenazine as described herein, such as the transdermal delivery described herein, can offer many advantages over traditional oral delivery, such as avoiding first-pass metabolism, providing a pharmacokinetic profile with low peak to trough ratio, providing capacity for multiple-day therapy from a single application, avoiding food effects on absorption, ceasing therapy by removing patch if necessary, and an easier patient compliance, etc. Further, compared to equal doses of oral administration of tetrabenazine or deutetrabenazine, the delivery herein such as the transdermal delivery as described herein can decrease inter-individual variation in plasma levels of tetrabenazine or deuterated tetrabenazine and/or a metabolite thereof, and/or can decrease Cₘₐₓ (e.g., by 10%, 40%, 60%, or more) of tetrabenazine or deuterated tetrabenazine and/or a metabolite thereof, for example, without also reducing therapeutic efficacy. Moreover, the delivery such as the transdermal delivery herein can be similarly administered to subjects who are genotyped based on CYP2D6 expression as poor metabolizer (PM), intermediate metabolizer (IM), or extensive metabolizer (EM). Further, unlike Xenazine® and Austedo™ treatments, the R,R-TBZ transdermal delivery system (TDDS) herein can be administered to subjects without the need to be genotyped before dosing (even at higher dose). As detailed herein, most of the Metabolizer effect is due to off target binding, which would not be expected from administering the R,R-TBZ TDDS herein. These advantages can ultimately lead to simplified dosing regimen for tetrabenazine and/or a deuterated tetrabenazine (e.g., deutetrabenazine), for example, the need to conduct genotype analysis can be minimized or eliminated, and/or reduced dose-related side effects.

In some embodiments, the present disclosure provides a method of treating a hyperkinetic movement disorder in a subject (e.g., a human subject) in need thereof. In some embodiments, the method comprises administering to the subject a pharmaceutical composition comprising an active ingredient comprising R,R-tetrabenazine, wherein the administering bypasses first-pass metabolism and delivers, such as continuously or substantially continuously delivers, to the subject about 0.1 mg/day to about 20 mg/day of R,R-tetrabenazine. In some embodiments, the pharmaceutical composition is administered transdermally, intravenously, subcutaneously, intramuscularly, or via a depot. In some specific embodiments, the administering bypasses first-pass metabolism and transdermally delivers to the subject about 0.1 mg/day to about 20 mg/day of R,R-tetrabenazine. In some embodiments, the administering comprises applying the pharmaceutical composition to the skin of the subject to transdermally deliver to the subject about 0.1 mg/day to about 20 mg/day of R,R-tetrabenazine. In some embodiments, the sole active ingredient in the pharmaceutical composition is a substantially pure R,R-isomer of tetrabenazine. In some embodiments, the administering can provide any of the applicable pharmacokinetic (PK) profile as described herein, for example, the PK profile relating to the plasma concentration profile described herein for R,R-tetrabenazine, R,R,R-dihydrotetrabenazine (HTBZ) and S,R,R-HTBZ. In some embodiments, the administering does not provide any detectable amount of S,S-isomer of tetrabenazine or metabolites thereof, including S,S,S-HTBZ and R,S,S-HTBZ, in the plasma of the subject. The pharmaceutical composition, drug delivery device, dosing regimen, and subject suitable for the method include any of those described herein. For example, in some embodiments, the pharmaceutical composition can be any of the adhesive compositions described herein, which can be included in any of the transdermal delivery device described herein. The pharmaceutical composition can be administered to the subject for as long as needed at any frequency. In some embodiments, each administration of the pharmaceutical composition can deliver, e.g., continuously or substantially continuously, to the subject about 0.1 mg/day to about 20 mg/day of R,R-tetrabenazine for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, the administering can be conducted without regard to the fed status of the subject. The ranges of doses and/or plasma exposures described herein can be suitable for adult patients and pediatric and adolescent patients. However, as understood by those skilled in the art, the desired doses and/or plasma exposures for a particular subject may be adjusted, considering the age and weight of the subject. In some embodiments, the subject is a pediatric and adolescent patient (e.g., 6 through 18 years of age). In some embodiments, the administering can be conducted without regard to the genotype of the subject, for example, the subject can be an extensive metabolizer. In some embodiments, the hyperkinetic movement disorder can be Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.

Some embodiments of the present disclosure are also directed to a method of treating a hyperkinetic movement disorder in a subject in need thereof, which comprises administering to the subject a pharmaceutical composition comprising an active ingredient comprising R,R-tetrabenazine, wherein the administering provides a desired PK profile. In some embodiments, the PK profile is characterized in that the administering provides a therapeutically effective plasma concentration of R,R-tetrabenazine, R,R,R-HTBZ and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the PK profile is characterized in that the administering provides a therapeutically effective plasma concentration of R,R-tetrabenazine, R,R,R-HTBZ and S,R,R-HTBZ, wherein the ratio of the steady state plasma concentration of R,R-tetrabenazine to the combined steady state plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the administering provides no detectable S,S-tetrabenazine, R,S,S-HTBZ, or S,S,S-HTBZ in the plasma of the subject. In some embodiments, the PK profile is characterized in that the plasma concentration of R,R-tetrabenazine rises during a first period of time to reach a maximum concentration for the first period of time of about 150 pg/ml to about 3500 pg/ml at a first time point, wherein the first period of time is from time 0 (the time of administration of the pharmaceutical composition) to about 24 hours thereafter. In some embodiments, the PK profile is characterized in that the average terminal half-life of R,R-tetrabenazine is about 8.5 hours±40% CV. In some embodiments, the PK profile is further characterized in that after the first period of time, the plasma concentration of R,R-tetrabenazine remains substantially constant for a sustained period of time, such as about 24 hours, about 48 hours, about 72 hours, about 96 hours, or more. In some embodiments, the PK profile is characterized in that the administering provides a substantially constant steady state plasma concentration of R,R-tetrabenazine above 150 pg/ml (e.g., about 150 pg/ml to about 3500 pg/ml), for a sustained period of at least 6 hours, or at least 12 hours, preferably, at least 24 hours. Other novel PK profiles are described herein. The pharmaceutical composition, drug delivery device, dosing regimen, and subject suitable for the method include any of those described herein. In some embodiments, the administering bypasses first-pass metabolism and delivers, such as continuously or substantially continuously delivers R,R-tetrabenazine to the subject, e.g., at a substantially constant rate. In some embodiments, the pharmaceutical composition is administered transdermally. In some embodiments, the pharmaceutical composition can be any of the adhesive compositions described herein. In some embodiments, the administering can be conducted without regard to the fed status of the subject. In some embodiments, the subject is a pediatric and adolescent patient (e.g., 6 through 18 years of age). In some embodiments, the administering can be conducted without regard to the genotype of the subject, for example, the subject can be an extensive metabolizer. In some embodiments, the hyperkinetic movement disorder can be Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.

Some embodiments of the present disclosure are directed to delivery, such as continuous or a substantially continuous delivery, of a deuterated tetrabenazine (e.g., R,R-deutetrabenazine), for example, for treating a hyperkinetic movement disorder. In some embodiments, the method comprises administering to the subject a pharmaceutical composition comprising an active ingredient comprising a deuterated R,R-tetrabenazine (e.g., R,R-deutetrabenazine), wherein the administering bypasses first-pass metabolism and delivers, such as continuously or substantially continuously delivers, to the subject about 0.1 mg/day to about 20 mg/day of deuterated R,R-tetrabenazine. In some embodiments, the pharmaceutical composition is administered transdermally, intravenously, subcutaneously, intramuscularly, or via a depot. In some specific embodiments, the administering bypasses first-pass metabolism and transdermally delivers to the subject about 0.1 mg/day to about 20 mg/day of deuterated R,R-tetrabenazine. In some embodiments, the administering comprises applying the pharmaceutical composition to the skin of the subject to transdermally deliver to the subject about 0.1 mg/day to about 20 mg/day of deuterated R,R-tetrabenazine. In some embodiments, the sole active ingredient in the pharmaceutical composition is a substantially pure R,R-isomer of deuterated tetrabenazine such as a substantially pure R,R-deutetrabenazine. In some embodiments, the administering does not provide any detectable amount of S,S-isomer of deuterated tetrabenazine and/or metabolites thereof in the plasma of the subject. In some embodiments, the administering can provide any of the applicable pharmacokinetic (PK) profile as described herein, for example, the PK profile relating to the plasma concentration profile described herein for deuterated R,R-tetrabenazine, deuterated R,R,R-HTBZ and deuterated S,R,R-HTBZ. The pharmaceutical composition, drug delivery device, dosing regimen, and subject suitable for the method include any of those described herein. In some embodiments, the pharmaceutical composition can be any of the adhesive compositions described herein. The pharmaceutical composition can be administered to the subject for as long as needed at any frequency. In some embodiments, each administration of the pharmaceutical composition can deliver, e.g., continuously or substantially continuously, to the subject about 0.1 mg/day to about 20 mg/day of deuterated R,R-tetrabenazine for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, the administering can be conducted without regard to the fed status of the subject. In some embodiments, the subject is a pediatric and adolescent patient (e.g., 6 through 18 years of age). In some embodiments, the administering can be conducted without regard to the genotype of the subject, for example, the subject can be an extensive metabolizer. In some embodiments, the hyperkinetic movement disorder can be Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In any of the embodiments described herein, the deuterated tetrabenazine can be deutetrabenazine, such as R,R-deutetrabenazine.

Some embodiments of the present disclosure are also directed to a method of treating a hyperkinetic movement disorder in a subject in need thereof, which comprises administering to the subject a pharmaceutical composition comprising an active ingredient comprising a deuterated R,R-tetrabenazine, wherein the administering provides a desired PK profile. In some embodiments, the PK profile is characterized in that the administering provides a therapeutically effective plasma concentration of deuterated R,R-tetrabenazine, deuterated R,R,R-HTBZ and deuterated S,R,R-HTBZ, wherein the ratio of the maximum plasma concentration of deuterated R,R-tetrabenazine to the combined maximum plasma concentration of deuterated R,R,R-HTBZ and deuterated S,R,R-HTBZ ranges from about 1:1 to about 1:7.5. In some embodiments, the PK profile is characterized in that the administering provides a therapeutically effective plasma concentration of deuterated R,R-tetrabenazine, deuterated R,R,R-HTBZ and deuterated S,R,R-HTBZ, wherein the ratio of the steady state plasma concentration of deuterated R,R-tetrabenazine to the combined steady state plasma concentration of deuterated R,R,R-HTBZ and deuterated S,R,R-HTBZ ranges from about 1:1 to about 1:7.5. In some embodiments, the administering provides no detectable deuterated S,S-tetrabenazine, deuterated R,S,S-HTBZ, or deuterated S,S,S-HTBZ in the plasma of the subject. In some embodiments, the PK profile is characterized in that the plasma concentration of deuterated R,R-tetrabenazine rises during a first period of time to reach a maximum concentration for the first period of time of about 150 pg/ml to about 3500 pg/ml at a first time point, wherein the first period of time is from time 0 (the time of administration of the pharmaceutical composition) to about 24 hours thereafter. In some embodiments, the PK profile is characterized in that the average terminal half-life of deuterated R,R-tetrabenazine is about 8.5 hr±40% CV. In some embodiments, the PK profile is further characterized in that after the first period of time, the plasma concentration of deuterated R,R-tetrabenazine remains substantially constant for a sustained period of time, such as about 24 hours, about 48 hours, about 72 hours, about 96 hours, or more. In some embodiments, the PK profile is characterized in that the administering provides a substantially constant steady state plasma concentration of deuterated R,R-tetrabenazine above 150 pg/ml (e.g., about 150 pg/ml to about 3500 pg/ml), for a sustained period of at least 6 hours, or at least 12 hours, preferably, at least 24 hours. Other novel PK profiles are described herein. The pharmaceutical composition, drug delivery device, dosing regimen, and subject suitable for the method include any of those described herein. In some embodiments, the administering bypasses first-pass metabolism and delivers, such as continuously or substantially continuously delivers deuterated R,R-tetrabenazine to the subject, e.g., at a substantially constant rate. In some embodiments, the pharmaceutical composition is administered transdermally. In some embodiments, the pharmaceutical composition can be any of the adhesive compositions described herein. In some embodiments, the administering can be conducted without regard to the fed status of the subject. In some embodiments, the subject is a pediatric and adolescent patient (e.g., 6 through 18 years of age). In some embodiments, the administering can be conducted without regard to the genotype of the subject, for example, the subject can be an extensive metabolizer. In some embodiments, the hyperkinetic movement disorder can be Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In any of the embodiments described herein, the deuterated tetrabenazine can be duetetrabenazine, such as R,R-deutetrabenazine.

Some specific embodiments of the present invention are directed to transdermal delivery of tetrabanazine or deuterated tetrabenazine to a subject in need thereof. The transdermal pharmaceutical composition and transdermal delivery device are also novel aspects of the present disclosure.

In some embodiments, the present invention provides a transdermal delivery device comprising tetrabenazine and/or a deuterated tetrabenazine (e.g., deutetrabenazine). In various embodiments, the present invention also provides a pharmaceutical composition (e.g., an adhesive composition) comprising tetrabenazine and/or a deuterated tetrabenazine (e.g., deutetrabenazine). In some embodiments, the present invention further provides a method of preparing or using a transdermal delivery device or a pharmaceutical composition comprising tetrabenazine and/or a deuterated tetrabenazine (e.g., deutetrabenazine).

Typically, the transdermal delivery device comprises a backing layer, a drug layer comprising a drug chosen from tetrabenazine, a deuterated tetrabenazine, or a combination thereof, for example, in an amount of about 2% to about 30% by weight of the drug layer, and an adhesive layer, which defines an active surface area. The transdermal delivery device is generally designed to have certain flux characteristics, for example, any of those defined herein. Typically, the transdermal delivery device can have an active surface area ranging from about 5 cm² to about 300 cm², for example, about 10 cm² to about 100 cm².

The transdermal delivery device herein is not limited to any specific patch designs. For example, the transdermal delivery device herein can be a drug-in-adhesive patch, drug-in-reservoir patch, micro-needle patch, or another patch design that may contain chemical or physical mode of enhancement. In some embodiments, the transdermal delivery device can be a drug-in-adhesive patch, for example, a single layer DIA patch. In some embodiments, the transdermal delivery device can comprise more than one drug layer, for example, two or more drug-in-adhesive layer. In some embodiments, the transdermal delivery device can be a drug-in-reservoir patch, for example, the drug layer is a reservoir comprising tetrabenazine and/or a deuterated tetrabenazine.

The drug layer can comprise tetrabenazine, deuterated tetrabenazine, or a combination thereof. In any of the embodiments described herein, the drug layer can be a drug-in-adhesive layer. In any of the embodiments described herein, the drug layer can comprise tetrabenazine, for example, a substantially pure R,R-tetrabenazine. In some embodiments, tetrabenazine (e.g., a substantially pure R,R-tetrabenazine) is the only active ingredient in the drug layer. In any of the embodiments described herein, the drug layer can comprise deutetrabenazine, for example, a substantially pure R,R-deutetrabenazine. In some embodiments, deutetrabenazine (e.g., a substantially pure R,R-deutetrabenazine) is the only active ingredient in the drug layer. In some embodiments, the drug layer comprises tetrabenazine, deuterated tetrabenazine, or a combination thereof, in an amount of about 2% to about 30% (e.g., about 2%, about 2.5%, about 5%, about 8%, about 10%, about 15%, about 18%, about 20%, about 25%, about 30%, or any ranges in between the recited values) by weight of the drug layer. In some specific embodiments, the drug layer comprises tetrabenazine, deuterated tetrabenazine, or a combination thereof, in the amount of about 2%, about 2.5%, about 5%, about 8%, about 10%, about 15%, or about 20% by weight of the drug layer. In some embodiments, the drug layer can optionally include one or more other ingredients, for example, selected from skin permeation enhancers, humectants, plasticizers, antioxidants, anti-irritants, gel-forming agents, drug release modifiers, solvents, crystallization inhibitors, and additional active ingredients. In some embodiments, the drug layer can have a coat weight of about 0.1 g/cm² to about 0.90 g/cm² (e.g., about 0.1 g/cm² to about 0.5 g/cm²) active surface area.

In some embodiments, the drug layer comprises tetrabenazine, deuterated tetrabenazine, or a combination thereof dispersed (e.g., homogenously dispersed) in an adhesive (e.g., a pressure sensitive adhesive). Suitable pressure sensitive adhesives are described herein. In some embodiments, the pressure sensitive adhesive can include a polyisobutylene (PIB) adhesive, a silicone polymer adhesive (e.g., Bio-7-4202), an acrylate copolymer adhesive (e.g., DuroTak 87-2287), or a combination thereof. In some embodiments, the pressure sensitive adhesive can be a non-reactive acrylate adhesive, for example, an acrylate adhesive that has no functional groups containing reactive hydrogen moieties, or an acrylate adhesive that has no functional groups selected from epoxy, —OH, —COOH, and combinations thereof.

The adhesive layer is typically formulated such that the transdermal delivery device can adhere to the skin of a user for a desired period of time. For example, in some embodiments, the transdermal delivery device is capable of adhering continuously to the skin of a user for about 8 hours, about 12 hours, about 18 hours, about 24 hours, about 2 days, about 3 days, about 4 days, about 5 days, about 6 days, or about 7 days or more.

Certain embodiments of the present invention are also directed to an adhesive composition. In some embodiments, the adhesive composition comprises a drug chosen from tetrabenazine, deuterated tetrabenazine (e.g., deutetrabenazine), and combinations thereof in an adhesive. In some embodiments, the drug is homogenously dispersed in the adhesive (e.g., a pressure sensitive adhesive). In some embodiments, the pressure sensitive adhesive can be a non-reactive acrylate adhesive, for example, an acrylate adhesive that has no functional groups containing reactive hydrogen moieties, or an acrylate adhesive that has no functional groups selected from epoxy, —OH, —COOH, and combinations thereof. In some embodiments, the pressure sensitive adhesive comprises a polyisobutylene (PIB) adhesive, a silicone polymer adhesive, an acrylate copolymer adhesive, or a combination thereof. In some embodiments, the adhesive composition comprises tetrabenazine (e.g., a substantially pure R,R-tetrabenazine) in an amount of about 2% to about 30% by weight of the adhesive composition. In some embodiments, the adhesive composition comprises deutetrabenazine (e.g., a substantially pure R,R-deutetrabenazine) in an amount of about 2% to about 30% by weight of the adhesive composition. In some embodiments, the active ingredient is present in an amount of about 2% to about 7% by weight. In some embodiments, the adhesive composition is free of a permeation enhancer, for example, free of isopropyl myristate. However, in some embodiments, the adhesive composition further comprises a permeation enhancer. In some embodiments, the adhesive composition can comprise an antioxidant, e.g., a gallate antioxidant, such as propyl gallate. In some embodiments, the adhesive composition can comprise a crystallization inhibitor, such as a polyvinylpyrrolidone polymer, a cross-linked polyvinylpyrrolidone polymer, a polyvinylpyrrolidone copolymer, a cellulose based polymer, a polycarboxylic acid polymer, a polymethacrylate, a polyethylene glycol, polyvinyl acetate and polyvinylcaprolactame-based graft copolymer (PVAc-PVCap-PEG), or a combination thereof. In some preferred embodiments, the adhesive composition comprises a crystallization inhibitor which is a copolymer of butyl methacrylate and methyl methacrylate. In some embodiments, the adhesive composition comprises a crystallization inhibitor which is a polyethylene glycol, polyvinyl acetate and polyvinylcaprolactame-based graft copolymer. In some embodiments, the adhesive composition is capable of adhering continuously to the skin of a user for an extended period of time selected from about 8 hours, about 12 hours, about 18 hours, about 24 hours, about 2 days, about 3 days, about 4 days, about 5 days, about 6 days, or about 7 days or more. The adhesive composition described herein can be used for a transdermal delivery device. For example, in some embodiments, the transdermal delivery device can include any of the adhesive compositions described herein, a backing layer, and a release liner.

In some embodiments, the present invention provides a method of transdermally administering tetrabenazine, deuterated tetrabenazine, or a combination thereof to a subject (e.g., human subject) in need thereof. In some embodiments, the method comprises applying any of the transdermal delivery devices or pharmaceutical compositions (e.g., adhesive compositions) to the subject, for example, to the skin of the subject. In some embodiments, the administering provides any of the desired daily dose described herein and/or any of the PK profiles provided herein (as applicable).

In some embodiments, the present invention also provides a method of inhibiting VMAT-2 in a subject in need thereof. In some embodiments, the method comprises applying any of the transdermal delivery devices or pharmaceutical compositions (e.g., adhesive compositions) to the subject, for example, to the skin of the subject. In some embodiments, the applying provides any of the desired daily dose described herein and/or any of the PK profiles provided herein (as applicable).

In some embodiments, the present invention also provides a method of treating a vesicular monoamine transporter isoform 2 (VMAT2) mediated disease or disorder in a subject (e.g., a human subject) in need thereof. In some embodiments, the method comprises applying any of the transdermal delivery devices or pharmaceutical compositions (e.g., adhesive compositions) to the subject, for example, to the skin of the subject. In some embodiments, the applying provides any of the desired daily dose described herein and/or any of the PK profiles provided herein (as applicable).

In some specific embodiments, the present invention provides a method of treating a hyperkinetic movement disorder in a subject (e.g., human subject) in need thereof. In some embodiments, the method comprises transdermally administering a therapeutically effective amount of tetrabenazine and/or deuterated tetrabenazine to the subject. In some embodiments, the method comprises applying any of the transdermal delivery devices or pharmaceutical compositions (e.g., adhesive compositions) to the subject, for example, to the skin of the subject. In some embodiments, the hyperkinetic movement disorder is a chronic hyperkinetic movement disorder. In some embodiments, the hyperkinetic movement disorder is chorea associated with Huntington's disease, Wilson's disease, Tourette syndrome, restless leg syndrome, tardive dyskinesia, and/or a tic. In some embodiments, the hyperkinetic movement disorder is chorea associated with Huntington's disease. In some embodiments, the applying provides any of the desired daily dose described herein and/or any of the PK profiles provided herein (as applicable).

In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism to deliver R,R-tetrabenazine to the subject continuously or substantially continuously, and identifying a pharmaceutical composition that provides any of the PK profiles described herein. In some embodiments, the method is for identifying a pharmaceutical composition suitable for transdermal delivery for treating a hyperkinetic movement disorder. In some embodiments, the method comprises measuring the skin flux characteristics of a test adhesive composition in vitro using human cadaver skin, and identifying an adhesive composition that provides any of the in vitro flux characteristics described herein.

### BRIEF DESCRIPTION OF THE DRAWINGS/FIGURES

FIG. 1 presents graphs showing the in vitro flux of tetrabenazine through human (Black) cadaver epidermis from monolithic matrices of Duro-Tak 87-2287 adhesive containing tetrabenazine at different concentrations (10%, 15%, 5%, and 2.5%).

FIG. 2 presents graphs showing the in vitro flux of tetrabenazine through human cadaver epidermis from monolithic matrices of a mixture of Duro-Tak 87-2287 adhesive and BIO-7-4202 at different ratios, including 5:95, 10:90, 25:75, and 50:50 (Duro-Tak 87-2287 to BIO-7-4202). All matrices for this figure contain 10% tetrabenazine.

FIG. 3 presents graphs showing in vitro flux of tetrabenazine through human cadaver epidermis from patch formulations prepared using DuroTak 87-900A.

FIG. 4A presents graphs showing the mean plasma concentration versus time plot. The combined tetrabenazine (R,R, and S,S-isomers were combined) concentrations were used for this graph. FIG. 4B presents graphs showing the mean plasma concentration of R,R,R-HTBZ versus time plot. FIG. 4C presents graphs showing the mean plasma concentration of S,R,R-HTBZ versus time plot. FIG. 4D presents graphs showing the mean plasma concentration of total tetrabenazine (including tetrabenazine, R,R,R-HTBZ, and S,R,R-HTBZ) versus time plot.

### DETAILED DESCRIPTION OF THE INVENTION

The FDA approved labels for Xenazine® and Austedo™ each contains a blackbox warning against potential risks of depression and suicidality. For both products, the actual dosing needs to be monitored and titrated. For example, the Xenazine® label indicates that for patients requiring doses above 50 mg per day, the patients should be genotyped the drug metabolizing enzyme CYP2D6 to determine if the patient is a poor metabolizer (PM) or an extensive metabolizer (EM). For poor metabolizers, the maximum daily dose can only be 50 mg, with the maximum single dose of 25 mg. Whereas for extensive metabolizers or intermediate metabolizers, the maximum daily dose is 100 mg, with the maximum single dose of 37.5 mg. Similarly, the Austedo™ label also states that for poor metabolizers, the maximum daily dose can only be 36 mg, with two single doses of 18 mg.

Despite recent advances, tetrabenazine/deutetrabenazine medication remains complicated, which involves dose titration to reduce potential dose-related side effects. Thus, novel tetrabenazine formulations and dosing options are needed.

In various embodiments, the present invention is directed to pharmaceutical compositions, drug delivery devices, methods of preparation, and methods of use such as those relate to delivery of tetrabenazine and/or a deuterated tetrabenazine to a subject bypassing first-pass metabolism, for example, in a continuous or substantially continuous fashion. For example, some embodiments of the present invention are directed to transdermal delivery of tetrabenazine and/or a deuterated tetrabenazine. Some embodiments of the present disclosure are directed to delivery of tetrabenazine and/or a deuterated tetrabenazine intravenously, subcutaneously, intramuscularly, or via a depot, such as continuously delivery, for example, at a substantially constant rate.

### Continuous Delivery of Tetrabenazine

Some embodiments of the present disclosure are based on the unique pharmacokinetic (PK) profile obtained from a continuous or substantially continuous delivery of tetrabenazine, transdermal delivery, which bypasses first-pass metabolism. As detailed herein, transdermal delivery of R,R-tetrabenazine at a dose of 8 mg/96 hours provides a much higher concentration of R,R-tetrabenazine, with no inter-conversion to the SS isomer, and a less extent of metabolism to the active HTBZ isomers with a lower dose compared to thrice-daily doses of TBZ tablets administered under fasted conditions from Day 1 through Day 4 (total dose of 150 mg) in healthy male subjects. This data first shows that a continuous or substantial continuous delivery method, such as transdermal delivery, can provide therapeutically effective plasma concentrations of R,R-tetrabenazine, and its dihydro-metabolites, R,R,R-HTBZ and S,R,R-HTBZ, for example, for treating a hyperkinetic movement disorder herein. As the R,R-isomer of tetrabenazine, R,R,R-HTBZ and S,R,R-HTBZ are believed to have a low off-target binding, such as towards the dopamine D1 or D2 receptors, using the R,R-isomer of tetrabenazine as the principal active ingredient will also cause less side effect compared to using the racemic mixture of tetrabenazine, which is the active ingredient of Xenazine®. Furthermore, the PK data shows that the continuous delivery method herein can provide a relatively stable plasma concentration of R,R-tetrabenazine and its active dihydro-metabolites, with a reduced Cₘₐₓ and reduced peak to trough ratio, compared to a dose equivalent oral delivery of tetrabenazine, and thus can be advantageous in treating a subject having a hyperkinetic movement disorder.

While the exemplified PK data herein is produced through the use of a transdermal delivery device, the present disclosure is not limited to transdermal delivery. Rather, for any of the methods described herein, the inventors specifically contemplate administering to a subject in need of treatment any tetrabenazine formulation or delivery device that produces a PK profile described herein. The PK profile described herein is one novel aspect of the present disclosure. In some embodiments, the PK profile can be achieved through applying the transdermal delivery device herein. In some embodiments, the PK profile can be achieved through any continuous or substantially continuous delivery means that bypasses first-pass metabolism, for example, a transdermal delivery, a continuous intravenous delivery, a subcutaneous delivery, an intramuscular delivery, or via a depot. These delivery methods share a common feature that each of which can be adjusted to systemically deliver tetrabenazine at a desired rate, for example, at a rate of about 0.1 mg/day to about 20 mg/day of R,R-tetrabenazine. In some embodiments, the delivery of tetrabenazine can be at a substantially constant rate during the drug delivery period, e.g., patch-on period, and the delivery of tetrabenazine to the subject resembles zero-order kinetic. For example, in some embodiments, the desired delivery rate can be about 12 mg/day of R,R-tetrabenazine, and the desired drug delivery period is 24 hours, then the amount of R,R-tetrabenazine delivered to the subject treated at each hour can be about 0.5 mg/hour. By “substantially continuous” as used in, for example, the context of “substantially continuous delivery” is meant to refer to delivery of drug (e.g., tetrabenazine) in a manner that is substantially uninterrupted for a pre-selected period of drug delivery. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery. The drug delivery period herein can vary, for example, from about 8 hours to about 192 hours, such as about 8 hours, about 12 hours, about 24 hours, about 36 hours, about 48 hours, about 72 hours, about 96 hours, about 120 hours, about 144 hours, about 168 hours, about 192 hours, or any range between the recited values. To be clear, the expression continuous delivery or substantially continuous delivery does not require that the active ingredient/drug be delivered to the subject continuously throughout the treatment; the expression only requires that during the period of drug delivery, the drug is delivered continuously or substantially continuously, as opposed to a bolus delivery. For example, when a pharmaceutical composition is administered once a day, it is permissible that there exists a period of time between two administrations where no drug is delivered to the subject treated.

Typically, the R,R-tetrabenazine is delivered to a subject in need thereof at a dose that is therapeutically effective in treating a hyperkinetic movement disorder described herein. In some embodiments, the method herein delivers to a subject in need of treatment of a hyperkinetic movement disorder herein about 0.1 mg/day to about 20 mg/day of R,R-tetrabenazine, such as about 0.1 mg/day, about 0.5 mg/day, about 1 mg/day, about 2 mg/day, about 3 mg/day, about 4 mg/day, about 5 mg/day, about 6 mg/day, about 7 mg/day, about 8 mg/day, about 10 mg/day, about 12 mg/day, about 14 mg/day, about 16 mg/day, about 18 mg/day, or about 20 mg/day of R,R-tetrabenazine, or any range between the recited values, for example, about 0.5 mg/day to about 10 mg/day of R,R-tetrabenazine, about 0.5 mg/day to about 8 mg/day of R,R-tetrabenazine, about 0.5 mg/day to about 6 mg/day of R,R-tetrabenazine, about 0.5 mg/day to about 4 mg/day of R,R-tetrabenazine, about 1 mg/day to about 10 mg/day of R,R-tetrabenazine, about 1 mg/day to about 8 mg/day of R,R-tetrabenazine, about 1 mg/day to about 6 mg/day of R,R-tetrabenazine, about 1 mg/day to about 4 mg/day of R,R-tetrabenazine, about 2 mg/day to about 10 mg/day of R,R-tetrabenazine, about 2 mg/day to about 8 mg/day of R,R-tetrabenazine, about 2 mg/day to about 6 mg/day of R,R-tetrabenazine, about 2 mg/day to about 4 mg/day of R,R-tetrabenazine, about 4 mg/day to about 10 mg/day of R,R-tetrabenazine, about 4 mg/day to about 8 mg/day of R,R-tetrabenazine, about 4 mg/day to about 6 mg/day of R,R-tetrabenazine, about 6 mg/day to about 10 mg/day of R,R-tetrabenazine, or about 6 mg/day to about 8 mg/day of R,R-tetrabenazine. In some preferred embodiments, the method delivers about 0.5 mg/day to about 8 mg/day of R,R-tetrabenazine or about 2 mg/day to about 6 mg/day of R,R-tetrabenazine. As used herein, the term R,R-tetrabenazine should be understood as encompassing R,R-tetrabenazine base, a pharmaceutically acceptable salt thereof, or a combination thereof. Typically, for a transdermal composition such as the adhesive composition described herein, the R,R-tetrabenazine can exist predominantly in its free base form. For example, in any of the embodiments described herein, the adhesive composition or drug-in-adhesive composition comprising the R,R-tetrabenazine can be prepared from mixing R,R-tetrabenazine free base with other ingredients, such as adhesives, antioxidants, crystallization inhibitors, etc. in the recited amount. The delivery of R,R-tetrabenazine to a subject herein should be understood as the amount of R,R-tetrabenazine delivered to the subject, e.g., the amount permeated the skin of the subject, which can be in any form, with the amount expressed as equivalent amount of R,R-tetrabenazine base. Other terms such as tetrabenazine, deuterated tetrabenazine, deuterated R,R-tetrabenazine, deutetrabenazine, R,R-deutetrabenazine, delivery of such to a subject, should be understood similarly.

In some embodiments, the R,R-tetrabenazine can be included in a pharmaceutical composition or a delivery device, e.g., a continuous delivery device, which can be configured to deliver to the subject at the dose described herein at a substantially constant rate. For example, in some embodiments, the daily dose of R,R-tetrabenazine can be about 0.5 mg/day to about 10 mg/day, and the continuous delivery device can be applied to a subject in need thereof once a day, which delivers R,R-tetrabenazine to the subject continuously or substantially continuously for a pre-selected time, for example, 12 hours, or 24 hours, and the rate of delivery of the R,R-tetrabenazine during the pre-selected time can be substantially the same. For the methods described herein, the pharmaceutical composition or delivery device comprising the R,R-tetrabenazine is not particularly limited and can be any of those suitable for delivering R,R-tetrabenazine at a desired dose at a desired rate.

While many of the exemplary embodiments are described as directed towards a method of treating a hyperkinetic movement disorder in a subject in need thereof, the present disclosure is not limited to such treatment method. Any of the delivery method of R,R-tetrabenazine and/or a deuterated R,R-tetrabenazine described herein can also be used for a method of inhibiting VMAT-2 in a subject in need thereof, a method of treating a hyperkinetic movement disorder in a subject (e.g., a human subject) in need thereof, and/or a method of treating a vesicular monoamine transporter isoform 2 (VMAT2) mediated disease or disorder in a subject (e.g., a human subject) in need thereof.

In some embodiments, the present disclosure provides a method of treating a hyperkinetic movement disorder in a subject in need thereof, the method comprising administering to the subject a pharmaceutical composition comprising an active ingredient comprising R,R-tetrabenazine, wherein the administering bypasses first-pass metabolism and continuously or substantially continuously delivers to the subject about 0.1 mg/day to about 20 mg/day of R,R-tetrabenazine, for example, about 0.5 mg/day to about 10 mg/day of R,R-tetrabenazine, about 0.5 mg/day to about 8 mg/day of R,R-tetrabenazine, about 0.5 mg/day to about 6 mg/day of R,R-tetrabenazine, about 0.5 mg/day to about 4 mg/day of R,R-tetrabenazine, about 1 mg/day to about 10 mg/day of R,R-tetrabenazine, about 1 mg/day to about 8 mg/day of R,R-tetrabenazine, about 1 mg/day to about 6 mg/day of R,R-tetrabenazine, about 1 mg/day to about 4 mg/day of R,R-tetrabenazine, about 2 mg/day to about 10 mg/day of R,R-tetrabenazine, about 2 mg/day to about 8 mg/day of R,R-tetrabenazine, about 2 mg/day to about 6 mg/day of R,R-tetrabenazine, about 2 mg/day to about 4 mg/day of R,R-tetrabenazine, about 4 mg/day to about 10 mg/day of R,R-tetrabenazine, about 4 mg/day to about 8 mg/day of R,R-tetrabenazine, about 4 mg/day to about 6 mg/day of R,R-tetrabenazine, about 6 mg/day to about 10 mg/day of R,R-tetrabenazine, or about 6 mg/day to about 8 mg/day of R,R-tetrabenazine. In some preferred embodiments, the method delivers about 0.5 mg/day to about 8 mg/day of R,R-tetrabenazine or about 2 mg/day to about 6 mg/day of R,R-tetrabenazine. The period of drug delivery herein can vary, for example, from about 8 hours to about 72 hours or more than 72 hours. The pharmaceutical composition can be administered to the subject for as long as needed at any frequency. For example, in some embodiments, each administration provides a continuously or substantially continuously delivery of R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, during the period of continuous or substantially continuous delivery, the R,R-tetrabenazine can be delivered to the subject at a substantially constant rate, for example, with the average rate of delivery for each hour substantially the same, such as within 80-125% of the overall average rate for the drug delivery period. In some embodiments, during the period of continuous or substantially continuous delivery, the R,R-tetrabenazine can be delivered to the subject at a different rate, for example, with the average rate of delivery for each hour being different. For example, in some embodiments, the average rate of delivery can be high initially but then decreases over the course of continuous or substantially continuous delivery. Typically, the precise delivery rate of R,R-tetrabenazine each hour is not critical, and those skilled in the art after reviewing the present disclosure would know how to select and design a dosing regimen to deliver a desired daily dose to the subject, such as about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine. In some embodiments, the delivery of the desired daily dose to the subject also provides a pharmacokinetic profile as described herein.

In some embodiments, the present disclosure provides a method of treating a hyperkinetic movement disorder in a subject in need thereof, the method comprising administering to the subject a pharmaceutical composition comprising an active ingredient comprising R,R-tetrabenazine, wherein the administering transdermally delivers to the subject about 0.1 mg/day to about 20 mg/day of R,R-tetrabenazine, for example, about 0.5 mg/day to about 10 mg/day of R,R-tetrabenazine, about 0.5 mg/day to about 8 mg/day of R,R-tetrabenazine, about 0.5 mg/day to about 6 mg/day of R,R-tetrabenazine, about 0.5 mg/day to about 4 mg/day of R,R-tetrabenazine, about 1 mg/day to about 10 mg/day of R,R-tetrabenazine, about 1 mg/day to about 8 mg/day of R,R-tetrabenazine, about 1 mg/day to about 6 mg/day of R,R-tetrabenazine, about 1 mg/day to about 4 mg/day of R,R-tetrabenazine, about 2 mg/day to about 10 mg/day of R,R-tetrabenazine, about 2 mg/day to about 8 mg/day of R,R-tetrabenazine, about 2 mg/day to about 6 mg/day of R,R-tetrabenazine, about 2 mg/day to about 4 mg/day of R,R-tetrabenazine, about 4 mg/day to about 10 mg/day of R,R-tetrabenazine, about 4 mg/day to about 8 mg/day of R,R-tetrabenazine, about 4 mg/day to about 6 mg/day of R,R-tetrabenazine, about 6 mg/day to about 10 mg/day of R,R-tetrabenazine, or about 6 mg/day to about 8 mg/day of R,R-tetrabenazine. In some preferred embodiments, the method delivers about 0.5 mg/day to about 8 mg/day of R,R-tetrabenazine or about 2 mg/day to about 6 mg/day of R,R-tetrabenazine. In some embodiments, the administering comprises applying the pharmaceutical composition to the skin of the subject. The exact location of the skin is not critical, although typically the pharmaceutical composition is applied to an intact area of skin, for example, the subject's outer arm. The period of drug delivery, in the context of transdermal patch can also be referred to as patch-on time, can vary, for example, from about 8 hours to about 72 hours or more than 72 hours. In some embodiments, the pharmaceutical composition is applied to the skin of the subject to continuously or substantially continuously deliver R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. The frequency of applications of the pharmaceutical composition to the subject for the methods herein can vary, such as once a day, once in more than a day, or once in a week, etc. In some embodiments, for each application, the pharmaceutical composition can be applied to the skin of the subject to adhere to the skin of the subject for about 8 hours, about 12 hours, about 24 hours, about 36 hours, about 48 hours, about 72 hours, about 96 hours, about 120 hours, about 144 hours, about 168 hours, about 192 hours, or any range between the recited values, which can deliver R,R-tetrabenazine continuously or substantially continuously to the subject during the patch-on period. In some embodiments, there is no lag time between applications of transdermal patches according to the dosing frequency herein. For example, in some embodiments, the transdermal delivery device is applied to the subject once a day, and each application of the transdermal delivery device can last (adhere to the skin of the subject) for about 24 hours before it is replaced with another transdermal delivery device which is typically substantially same. However, in some embodiments, there can also be overlap or lag between two applications.

Typically, for transdermal delivery, the pharmaceutical composition can include an adhesive composition, e.g., in a transdermal delivery device (e.g., described herein), which comprises the active ingredient dispersed in an adhesive (preferably a pressure sensitive adhesive). In some embodiments, the adhesive composition is applied to the subject to deliver about 0.1 mg/day to about 20 mg/day of R,R-tetrabenazine (e.g., any of the exemplified ranges described herein) at a substantially constant rate for up to 24 hours post application, up to 48 hours post application, up to 96 hours post application, or up to 1 week post application. Suitable adhesive composition include any of those described herein, e.g., any of those described in exemplary Embodiments 1-18 as applicable to tetrabenazine or any of the specific compositions shown in the Examples section (e.g., Example 4A). In some embodiments, the adhesive composition comprises the active ingredient dispersed in a non-reactive acrylate pressure sensitive adhesive. The active ingredient can be present in an amount of about 1% to about 20% by weight, such as about 1%, about 2%, about 3%, about 4%, about 5%, about 6%, about 7%, about 10%, about 15%, or about 20% by weight, or any ranges between the recited values, for example, about 1% to about 15%, about 2% to about 15%, about 2% to about 10%, about 2% to about 7%, about 3% to about 15%, about 3% to about 10%, about 3% to about 7%, about 5% to about 15%, about 5% to about 10%, about 5% to about 7%, about 7% to about 15%, about 7% to about 10%, about 10% to about 20%, about 10% to about 15%, etc. by weight. In some embodiments, the adhesive composition comprises a substantially pure R,R-isomer of tetrabenazine as the sole active ingredient. In some embodiments, the substantially pure R,R-isomer of tetrabenazine is in a free base form. Suitable adhesive include any of those described herein, such as any of the pressure sensitive adhesive described herein. In some embodiments, the adhesive can be a non-reactive acrylate pressure sensitive adhesive described herein, such as Duro-Tak 87-900A or described in exemplary Embodiments 2-7. The adhesive typically is present in an amount of about 50% to about 97% by weight, such as about 50%, about 60%, about 70%, about 80%, about 90%, about 95%, or about 97% by weight, or any ranges between the recited values, for example, about 50% to about 95%, about 50% to about 90%, about 50% to about 80%, about 50% to about 70%, about 50% to about 60%, about 60% to about 97%, about 60% to about 95%, about 60% to about 90%, about 60% to about 80%, about 60% to about 70%, about 70% to about 97%, about 70% to about 95%, about 70% to about 90%, about 70% to about 80%, about 80% to about 97%, about 80% to about 95%, about 80% to about 90%, etc. by weight. In some specific embodiments, the active ingredient is in an amount of about 2% to about 7% by weight and the non-reactive acrylate pressure sensitive adhesive is in an amount of about 50% to about 97% by weight. The adhesive composition can also optionally include other ingredients, such as an antioxidant, a crystallization inhibitor, a plasticizer, and/or a permeation enhancer. In some embodiments, the adhesive composition includes an antioxidant, such as a gallate antioxidant, for example, propyl gallate. The amount of antioxidant is typically included in an amount of about 0% to about 1% by weight, such as about 0.001%, about 0.01%, about 0.1%, about 0.5%, about 1%, or any ranges between the recited values, for example, about 0.001% to about 0.5%, about 0.01% to about 0.5%, etc. by weight. In some embodiments, the adhesive composition includes a crystallization inhibitor, for example, a means to prevent formation of drug crystals (crystals of the active ingredient of the adhesive composition, such as R,R-tetrabenazine) after shelf storage for two weeks at ambient temperature. In some embodiments, the adhesive composition includes a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. In some embodiments, the adhesive composition includes a crystallization inhibitor selected from a polymethacrylate (e.g., Plastoid B (copolymer of butyl methacrylate and methyl methacrylate), Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. The crystallization inhibitor is typically present in an amount of about 0 to about 40% by weight, such as about 5%, about 10%, about 15%, about 20%, about 30%, about 40%, or any range between the recited values, for example, about 10% to about 40%, about 10% to about 30%, about 10% to about 20%, 15% to about 40%, about 15% to about 30%, about 15% to about 20%, 20% to about 40%, about 20% to about 30%, etc. by weight. In some embodiments, the adhesive composition can also comprise a skin permeation enhancer as described herein, such as isopropyl myristate. In some embodiments, the adhesive composition can also be free of a skin permeation enhancer as described herein, for example, in some embodiments, the adhesive composition can also be free of isopropyl myristate. Suitable drug loadings, active surface area, thickness, adhesive properties, etc. include any of those described herein in any combinations.

In some embodiments, the present disclosure provides a method of treating a hyperkinetic movement disorder in a subject in need thereof, the method comprising administering to the subject a pharmaceutical composition comprising an active ingredient comprising R,R-tetrabenazine, wherein the administering is via an injection or infusion such as an intravenous injection (not including only a bolus injection for immediate release), subcutaneous injection, or intramuscular injection, which delivers to the subject about 0.1 mg/day to about 20 mg/day of R,R-tetrabenazine, for example, about 0.5 mg/day to about 10 mg/day of R,R-tetrabenazine, about 0.5 mg/day to about 8 mg/day of R,R-tetrabenazine, about 0.5 mg/day to about 6 mg/day of R,R-tetrabenazine, about 0.5 mg/day to about 4 mg/day of R,R-tetrabenazine, about 1 mg/day to about 10 mg/day of R,R-tetrabenazine, about 1 mg/day to about 8 mg/day of R,R-tetrabenazine, about 1 mg/day to about 6 mg/day of R,R-tetrabenazine, about 1 mg/day to about 4 mg/day of R,R-tetrabenazine, about 2 mg/day to about 10 mg/day of R,R-tetrabenazine, about 2 mg/day to about 8 mg/day of R,R-tetrabenazine, about 2 mg/day to about 6 mg/day of R,R-tetrabenazine, about 2 mg/day to about 4 mg/day of R,R-tetrabenazine, about 4 mg/day to about 10 mg/day of R,R-tetrabenazine, about 4 mg/day to about 8 mg/day of R,R-tetrabenazine, about 4 mg/day to about 6 mg/day of R,R-tetrabenazine, about 6 mg/day to about 10 mg/day of R,R-tetrabenazine, or about 6 mg/day to about 8 mg/day of R,R-tetrabenazine. In some preferred embodiments, the method delivers about 0.5 mg/day to about 8 mg/day of R,R-tetrabenazine or about 2 mg/day to about 6 mg/day of R,R-tetrabenazine. In some embodiments, the administering comprises continuous or substantially continuous intravenous injection or infusion of the pharmaceutical composition to the subject. The period of drug delivery can vary, for example, from about 8 hours to about 72 hours or more than 72 hours. For example, in some embodiments, the pharmaceutical composition is intravenously injected or infused to the subject to continuously or substantially continuously deliver R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. The rate of such intravenous injection or infusion can be controlled for example, by a pump and adjusted to deliver R,R-tetrabenazine to the subject at a substantially constant rate for the drug delivery period, such as the infusion period. In some embodiments, the administering comprises a subcutaneous or intramuscular injection of the pharmaceutical composition to the subject. Typically, for subcutaneous or intramuscular injection, the pharmaceutical composition is formulated to release R,R-tetrabenazine to the subject for systemic absorption for a pre-selected period, for example, from about 8 hours to about 72 hours or more than 72 hours. For example, in some embodiments, the subcutaneous or intramuscular pharmaceutical composition is formulated or included in a drug delivery device such as an implant to continuously or substantially continuously release R,R-tetrabenazine to the subject for systemic absorption for about 8 hours, about 12 hours, about 24 hours, about 36 hours, about 48 hours, about 72 hours, about 96 hours, about 120 hours, about 144 hours, about 168 hours, about 192 hours, or any range between the recited values. Suitable implants include any of those known in the art for subcutaneous or intramuscular delivery of pharmaceutical agents, such as passive polymeric implants, including biodegradable and non-biodegradable implants, or active implants, such as osmotic pressure gradients and electromechanical drives. Non-limiting examples of formulating an implant for subcutaneous delivery include those described in U.S. Pat. No. 8,921,387 (buprenorphine), U.S. Pat. No. 6,835,194 (fentanyl), etc. See also, Stewart S. A., et al. “Implantable polymeric drug delivery devices, classification, manufacture, materials, and clinical applications,” Polymers, 10:1379 (2018). In some embodiments, the pharmaceutical composition is formulated as a long acting injectable formulation, such as a microsphere, which can be configured to release R,R-tetrabenazine to the subject for systemic absorption for a pre-selected period, for example, from about 8 hours to about 72 hours or more than 72 hours. In some embodiments, the long acting injectable formulation can be configured to release R,R-tetrabenazine to the subject for systemic absorption at a substantially constant rate for the pre-selected period to deliver the desired daily dose as described herein.

The hyperkinetic movement disorder recited in the methods of treatment herein include any of those known to be treatable by tetrabenazine or deutetrabenazine, for example, any of the approved indications by any regulatory agency including the U.S. FDA or those under clinical investigations. Non-limiting examples include Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In some embodiments, the hyperkinetic movement disorder can be Huntington's disease, such as chorea associated with Huntington's disease. In some embodiments, the hyperkinetic movement disorder can be Wilson's disease. In some embodiments, the hyperkinetic movement disorder can be Tourette syndrome. In some embodiments, the hyperkinetic movement disorder can be restless leg syndrome. In some embodiments, the hyperkinetic movement disorder can be tardive dyskinesia. In some embodiments, the hyperkinetic movement disorder can be tic.

In some embodiments, the hyperkinetic movement disorder can be dyskinetic cerebral palsy. Cerebral palsy (“CP”) refers to a group of neurological disorders that appear in infancy or early childhood and permanently affect body movement and muscle coordination. CP is caused by damage to or abnormalities inside the developing brain that disrupt the brain's ability to control movement and maintain posture and balance. The signs of CP usually appear in the early months of life, although specific diagnosis may be delayed until the age of 2 years or older. Currently, there is no approved treatment available for Dyskinesia in cerebral palsy (DCP). The available treatment options address some of the manifestations of DCP. Teva is currently conducting Phase III clinical trials using Deutetrabenazine. According to Teva, the study population will include pediatric and adolescent patients (6 through 18 years of age) with DCP with predominant choreiform movement disorder, who have had nonprogressive CP symptoms since infancy (≤2 years of age). The inventors believe that the methods and compositions herein can provide alternative and/or superior treatment options for dyskinetic cerebral palsy.

In some embodiments, the hyperkinetic movement disorder is a dystonia or dyskinesia disorder. See e.g., for a review of treatment of dystonia in Thengnatt M. A. and Jankovic J. neurotherapeutics, 11(1): 139-152 (2014).

The methods of treatment herein are not limited to any specific type of subjects. For example, the method herein can be administered to the subject without regard to the fed status of the subject. In other words, fed and fast status of the subject is not critical for the methods herein. In some embodiments, the subject is a pediatric and adolescent patient (e.g., 6 through 18 years of age). Also, in some embodiments, the method is not limited to any specific genotyped subjects. In some embodiments, the same dose or substantially the same dose of R,R-tetrabenazine can be administered to subjects who are characterized as PM, IM, or EM. In some embodiments, the subject is characterized as EM. In some embodiments, the subject is characterized as PM. In some embodiments, the subject is characterized as IM. In some embodiments, the method does not require dose titration and/or genotype analysis, which is required when treated with either of Xenazine® and Austedo™ tablets. In some embodiments, any of the methods herein can be adapted for treating a pediatric and adolescent patient (e.g., 6 through 18 years of age) having any of the diseases or disorders herein. As discussed herein, the ranges of doses and/or plasma exposures described herein can be suitable for adult patients and pediatric and adolescent patients. However, as understood by those skilled in the art, the desired doses and/or plasma exposures for pediatric and adolescent patients may be adjusted, typically to a lower dosage or exposure, considering the age and weight of the subject. Without wishing to be bound by theories, the convenient and controlled delivery of R,R-tetrabenazine herein can in some embodiments provide superior safety and treatment option over currently available treatments.

The dosing regimen for the methods of treatment herein is not particularly limited, so long as the desired dose of R,R-tetrabenazine is delivered to the subject for a desired period of time at a desired rate, which includes any of those described herein. For example, in some embodiments, the pharmaceutical composition is administered to the subject once a day. In some embodiments, the pharmaceutical composition is administered to the subject once in more than a day, such as once in two days, once in three days, once in four days, once in five days, once in six days, once in a week, or once in more than a week. In some embodiments, each administration provides a continuous or substantially continuous delivery of R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, there can be no or substantially no lag time between two consecutive administrations, such that the R,R-tetrabenazine is continued to be delivered to the subject according to the dosing regimen herein. In some embodiments, there can also be overlap or lag time between two consecutive administrations.

### Pharmacokinetics of Continuous Delivery of Tetrabenazine

Some embodiments of the present disclosure are also directed to a method of treating a hyperkinetic movement disorder in a subject in need thereof, which comprises delivering to the subject a therapeutically effective amount of R,R-tetrabenazine, wherein the delivering provides certain novel pharmacokinetic profile in the plasma of the subject.

As discussed herein, the current approved tetrabenazine or deutetetrabenazine products, such as Xenazine® and Austedo™, require complicated dose titration and require genotype analysis of treated subjects at a higher dose. Further, oral administration of such products also results in a high peak to trough ratio, with significant inter-individual variations in plasma levels of tetrabenazine or deutetrabenazine or a metabolite thereof. Typically, as also shown herein, R,R-tetrabenazine itself, which is a potent VMAT-2 inhibitor, is not present in circulation in any significant amount after oral administration. In some aspects, the inventors first show that by using the continuous or substantially continuous delivery of tetrabenazine herein, the average terminal half-life of R,R-tetrabenazine, administered by first-pass bypass route, can be around 8.5 hr±40% CV. In comparison, using the typical oral administration, R,R-tetrabenazine is typically not available systemically.

In various embodiments, the continuous or substantially continuous delivery of tetrabenazine described herein that achieves the novel pharmacokinetic profile herein can be advantageously used to deliver tetrabenazine for example, for the treatment of a hyperkinetic movement disorder.

In some embodiments, the present disclosure provides a method of treating a hyperkinetic movement disorder in a subject in need thereof, the method comprising administering to the subject a pharmaceutical composition comprising an active ingredient comprising R,R-tetrabenazine, wherein the administering provides a desired pharmacokinetic profile (PK profile). For example, in some embodiments, the desired PK profile is characterized in that the administering provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.), i.e., Cₘₐₓ of R,R-tetrabenazine/(Cₘₐₓ of R,R,R-HTBZ+Cₘₐₓ of S,R,R-HTBZ) ranges from about 1:1 to about 1:5. As used herein, in some embodiments, the term “maximum plasma concentration” can refer to the maximum plasma concentration obtained after a single dose administration of the pharmaceutical composition, e.g., an application of a single transdermal patch herein for delivery of 1 day, 2 days, 3 days, or more. As used herein, in some embodiments, the term “maximum plasma concentration” can also refer to the maximum plasma concentration obtained after multiple doses administrations of the pharmaceutical composition, preferably, the maximum plasma concentration obtained after steady state is reached. However, it should be understood that the ratios herein should be based on comparisons of concentrations of the same type (e.g., from single administration, steady state, etc.) among the respective compounds and the ratios can be said to fall within the recited respective ranges herein when calculated based on (1) single dose administration Cₘₐₓ and/or (2) multiple doses administration Cₘₐₓ (preferably, steady state Cₘₐₓ). In some embodiments, the desired PK profile is characterized in that the administering provides a ratio of the steady state plasma concentration of R,R-tetrabenazine to the combined steady state plasma concentration of R,R,R-HTBZ and S,R,R-HTBZ ranges from about 1:1 to about 1:5, i.e., Cₛₛ of R,R-tetrabenazine/(Cₛₛ of R,R,R-HTBZ+Cₛₛ of S,R,R-HTBZ) ranges from about 1:1 to about 1:5. In a continuous or substantially continuous delivery herein, steady state can be typically reached and maintained for a desired period of time. For example, in some embodiments, a steady state can be reached through application of a single transdermal patch herein for about 32-48 hours or more, which steady state can be maintained either with the same patch or through replacement at any frequency of the transdermal patch with a new patch that can deliver R,R-tetrabenazine at substantially the same rate. In some embodiments, a steady state is not reached through a single application of a transdermal patch herein, but can be reached through multiple applications of transdermal patches sequentially. As would be understood by those skilled in the art, at steady state, drug concentrations obtained from transdermal delivery patches can be substantially constant, in other words, the PK curve at steady state can be substantially flat or plateaued for a period of time. As used herein, steady state of R,R-TBZ refers to the situation where the overall intake of R,R-TBZ is fairly in dynamic equilibrium with its elimination. Steady state of R,R,R-HTBZ refers to the situation where the overall generation of R,R,R-HTBZ is fairly in dynamic equilibrium with its elimination. Steady state of S,R,R-HTBZ refers to the situation where the overall generation of S,R,R-HTBZ is fairly in dynamic equilibrium with its elimination. To be clear, as used herein, the ratios of steady state concentrations of R,R-tetrabenazine, R,R,R-dihydrotetrabenazine (HTBZ) and/or S,R,R-HTBZ can be said to be within the recited ranges herein when calculated (1) based on concentrations of the respective compounds at any time point when all three compounds, R,R-tetrabenazine, R,R,R-dihydrotetrabenazine (HTBZ) and S,R,R-HTBZ, are at a steady state, and/or (2) based on the average concentrations of the respective compounds during a continuous period of time when all three compounds, R,R-tetrabenazine, R,R,R-dihydrotetrabenazine (HTBZ) and S,R,R-HTBZ, are at a steady state. Ratios of steady state concentrations of other compounds herein should be understood similarly. As used herein, a therapeutically effective plasma concentration of R,R-tetrabenazine, R,R,R-dihydrotetrabenazine (HTBZ) and S,R,R-HTBZ does not require each of the three compounds to be in a therapeutically effective plasma concentration by itself. It suffices that the three compounds in combination are therapeutically effective, for example, at its respective steady state concentrations. In some embodiments, the ratio of maximum plasma concentration or steady state plasma concentration of R,R,R-HTBZ to S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments, the ratio of maximum plasma concentration of R,R-tetrabenazine:R,R,R-HTBZ:S,R,R-HTBZ can range from about 17-40:3-10:50-80. In some embodiments, the ratio of steady state plasma concentration of R,R-tetrabenazine:R,R,R-HTBZ:S,R,R-HTBZ can range from about 17-40:3-10:50-80. In some embodiments, the administering can provide a pharmacokinetic profile characterized in that the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5. In some embodiments, the desired PK profile can also be characterized in that the administering provides a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the desired PK profile can also be characterized in that the administering provides a substantially constant steady state plasma concentration of R,R-tetrabenazine above 150 pg/ml (e.g., about 150 pg/ml to about 3000 pg/ml), for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the administering provides no detectable S,S-tetrabenazine, R,S,S-HTBZ, or S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism to deliver R,R-tetrabenazine to the subject continuously or substantially continuously, and identifying a pharmaceutical composition that provides any of the PK profiles described in this paragraph in any combination.

In some embodiments, the pharmaceutical composition is formulated to provide a dose of about 2 mg/day of R,R-tetrabenazine for 4 days when administered once. In some embodiments, the method comprises administering the pharmaceutical composition to provide a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ S,R,R-HTBZ later than the mean Tₘₐₓ of R,R-tetrabenazine;
c) a mean Cₘₐₓ of R,R-tetrabenazine of about 300 pg/ml to about 700 pg/ml;
d) a mean Cₘₐₓ of R,R,R-HTBZ of about 60 pg/ml to about 200 pg/ml;
e) a mean Cₘₐₓ of S,R,R-HTBZ of about 1000 pg/ml to about 3000 pg/ml;
f) a mean AUC₀₋₉₆ of R,R-tetrabenazine of about 20 ng*h/ml to about 50 ng*h/ml;
g) a mean AUC₀₋₉₆ of R,R,R-HTBZ of about 4 ng*h/ml to about 12 ng*h/ml; and/or
h) a mean AUC₀₋₉₆ of S,R,R-HTBZ of about 70 ng*h/ml to about 200 ng*h/ml.
   In some embodiments, the pharmacokinetic profile in the subject is characterized by any one of a) to h). In some embodiments, the pharmacokinetic profile in the subject is characterized by a) and b). In some embodiments, the pharmacokinetic profile in the subject is characterized by c), d), and e). In some embodiments, the pharmacokinetic profile in the subject is characterized by f), g), and h). In some embodiments, the pharmacokinetic profile in the subject is characterized by 1) a) and/or b); 2) c), d), and/or e); and 3) f), g), and/or h). In some embodiments, the pharmacokinetic profile in the subject is characterized by all of a) to h). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the steady state plasma concentration of R,R-tetrabenazine to the combined steady state plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the ratio of maximum plasma concentration or steady state plasma concentration of R,R,R-HTBZ to S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments, the ratio of maximum plasma concentration of R,R-tetrabenazine:R,R,R-HTBZ:S,R,R-HTBZ can range from about 17-40:3-10:50-80. In some embodiments, the ratio of steady state plasma concentration of R,R-tetrabenazine:R,R,R-HTBZ:S,R,R-HTBZ can range from about 17-40:3-10:50-80. In some embodiments, the administering can provide a pharmacokinetic profile characterized in that the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5. In some embodiments, the desired PK profile can also be characterized in that the administering provides a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the desired PK profile can also be characterized in that the administering provides a substantially constant steady state plasma concentration of R,R-tetrabenazine above 150 pg/ml (e.g., about 150 pg/ml to about 3000 pg/ml), for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the administering provides no detectable S,S-tetrabenazine, R,S,S-HTBZ, or S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism, and identifying a pharmaceutical composition that when administered to provide a dose of about 2 mg/day for 4 days, provides any of the PK profiles described in this paragraph in any combination.

In some embodiments, the pharmaceutical composition is formulated to provide a dose of about 4-6 mg/day of R,R-tetrabenazine for at least one day, for example, two days, three days, four days, or a week, when administered once. In some embodiments, the method comprises administering the pharmaceutical composition to provide a pharmacokinetic profile in the subject characterized by:
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 600 pg/ml to about 2100 pg/ml;
d) a mean Cₘₐₓ of R,R,R-HTBZ of about 120 pg/ml to about 600 pg/ml;
e) a mean Cₘₐₓ of S,R,R-HTBZ of about 2000 pg/ml to about 9000 pg/ml;
f) a mean AUC₀₋₉₆ of R,R-tetrabenazine of about 40 ng*h/ml to about 150 ng*h/ml;
g) a mean AUC₀₋₉₆ of R,R,R-HTBZ of about 8 ng*h/ml to about 36 ng*h/ml; and/or
h) a mean AUC₀₋₉₆ of S,R,R-HTBZ of about 140 ng*h/ml to about 600 ng*h/ml.
   In some embodiments, each administration of the pharmaceutical composition provides a dose of about 4-6 mg/day of R,R-tetrabenazine for one day. In some embodiments, each administration of the pharmaceutical composition provides a dose of about 4-6 mg/day of R,R-tetrabenazine for two days. In some embodiments, each administration of the pharmaceutical composition provides a dose of about 4-6 mg/day of R,R-tetrabenazine for three days. In some embodiments, each administration of the pharmaceutical composition provides a dose of about 4-6 mg/day of R,R-tetrabenazine for four days. In some embodiments, each administration of the pharmaceutical composition provides a dose of about 4-6 mg/day of R,R-tetrabenazine for 1 week. In some embodiments, the pharmaceutical composition is an adhesive composition described herein, e.g., included in a transdermal delivery patch described herein, and the pharmaceutical composition can be administered to adhere to the skin of the subject for the desired period of R,R-tetrabenazine delivery, such as one day, two days, three days, four days, one week, or any range in between. Other pharmaceutical compositions for continuous delivery herein can be administered according to normal practice with respect to such composition to achieve the desired period of R,R-tetrabenazine delivery, for example, a depot or an implant can be formulated to release about 4-6 mg/day of R,R-tetrabenazine for the desired period once administered (e.g., injected) to the subject. In some embodiments, the pharmacokinetic profile in the subject is characterized by any one of a) to h). In some embodiments, the pharmacokinetic profile in the subject is characterized by a) and b). In some embodiments, the pharmacokinetic profile in the subject is characterized by c), d), and e). In some embodiments, the pharmacokinetic profile in the subject is characterized by f), g), and h). In some embodiments, the pharmacokinetic profile in the subject is characterized by 1) a) and/or b); 2) c), d), and/or e); and 3) f), g), and/or h). In some embodiments, the pharmacokinetic profile in the subject is characterized by all of a) to h). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the steady state plasma concentration of R,R-tetrabenazine to the combined steady state plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the ratio of maximum plasma concentration or steady state plasma concentration of R,R,R-HTBZ to S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments, the ratio of maximum plasma concentration of R,R-tetrabenazine:R,R,R-HTBZ:S,R,R-HTBZ can range from about 17-40:3-10:50-80. In some embodiments, the ratio of steady state plasma concentration of R,R-tetrabenazine:R,R,R-HTBZ:S,R,R-HTBZ can range from about 17-40:3-10:50-80. In some embodiments, the administering can provide a pharmacokinetic profile characterized in that the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5. In some embodiments, the desired PK profile can also be characterized in that the administering provides a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the desired PK profile can also be characterized in that the administering provides a substantially constant steady state plasma concentration of R,R-tetrabenazine above 150 pg/ml (e.g., about 150 pg/ml to about 3000 pg/ml), for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the administering provides no detectable S,S-tetrabenazine, R,S,S-HTBZ, or S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism, and identifying a pharmaceutical composition that when administered to provide a dose of about 4-6 mg/day for at least one day, for example, two days, three days, four days, or a week, provides any of the PK profiles described in this paragraph in any combination.

In some embodiments, the pharmaceutical composition is formulated to provide a dose of about 1-10 mg/day of R,R-tetrabenazine for at least one day, for example, two days, three days, four days, or a week, when administered once. In some embodiments, the method comprises administering the pharmaceutical composition to provide a pharmacokinetic profile in the subject characterized by:
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 150 pg/ml to about 3500 pg/ml;
d) a mean Cₘₐₓ of R,R,R-HTBZ of about 30 pg/ml to about 1000 pg/ml;
e) a mean Cₘₐₓ of S,R,R-HTBZ of about 500 pg/ml to about 15 ng/ml;
f) a mean AUC₀₋₉₆ of R,R-tetrabenazine of about 10 ng*h/ml to about 250 ng*h/ml;
g) a mean AUC₀₋₉₆ of R,R,R-HTBZ of about 2 ng*h/ml to about 60 ng*h/ml; and/or
h) a mean AUC₀₋₉₆ of S,R,R-HTBZ of about 35 ng*h/ml to about 1000 ng*h/ml.
   In some embodiments, each administration of the pharmaceutical composition provides a dose of about 1-10 mg/day of R,R-tetrabenazine for one day. In some embodiments, each administration of the pharmaceutical composition provides a dose of about 1-10 mg/day of R,R-tetrabenazine for two days. In some embodiments, each administration of the pharmaceutical composition provides a dose of about 1-10 mg/day of R,R-tetrabenazine for three days. In some embodiments, each administration of the pharmaceutical composition provides a dose of about 1-10 mg/day of R,R-tetrabenazine for four days. In some embodiments, each administration of the pharmaceutical composition provides a dose of about 1-10 mg/day of R,R-tetrabenazine for 1 week. In some embodiments, the pharmaceutical composition is an adhesive composition described herein, e.g., included in a transdermal delivery patch described herein, and the pharmaceutical composition can be administered to adhere to the skin of the subject for the desired period of R,R-tetrabenazine delivery, such as one day, two days, three days, four days, one week, or any range in between. Other pharmaceutical compositions for continuous delivery herein can be administered according to normal practice with respect to such composition to achieve the desired period of R,R-tetrabenazine delivery, for example, a depot or an implant can be formulated to release about 1-10 mg/day of R,R-tetrabenazine for the desired period once administered (e.g., injected) to the subject. In some embodiments, the pharmacokinetic profile in the subject is characterized by any one of a) to h). In some embodiments, the pharmacokinetic profile in the subject is characterized by a) and b). In some embodiments, the pharmacokinetic profile in the subject is characterized by c), d), and e). In some embodiments, the pharmacokinetic profile in the subject is characterized by f), g), and h). In some embodiments, the pharmacokinetic profile in the subject is characterized by 1) a) and/or b); 2) c), d), and/or e); and 3) f), g), and/or h). In some embodiments, the pharmacokinetic profile in the subject is characterized by all of a) to h). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ, wherein the ratio of the steady state plasma concentration of R,R-tetrabenazine to the combined steady state plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments, the ratio of maximum plasma concentration or steady state plasma concentration of R,R,R-HTBZ to S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments, the ratio of maximum plasma concentration of R,R-tetrabenazine:R,R,R-HTBZ:S,R,R-HTBZ can range from about 17-40:3-10:50-80. In some embodiments, the ratio of steady state plasma concentration of R,R-tetrabenazine:R,R,R-HTBZ:S,R,R-HTBZ can range from about 17-40:3-10:50-80. In some embodiments, the administering can provide a pharmacokinetic profile characterized in that the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5. In some embodiments, the desired PK profile can also be characterized in that the administering provides a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the desired PK profile can also be characterized in that the administering provides a substantially constant steady state plasma concentration of R,R-tetrabenazine above 150 pg/ml (e.g., about 150 pg/ml to about 3000 pg/ml), for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the administering provides no detectable S,S-tetrabenazine, R,S,S-HTBZ, or S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism, and identifying a pharmaceutical composition that when administered to provide a dose of about 1-10 mg/day for at least one day, for example, two days, three days, four days, or a week, provides any of the PK profiles described in this paragraph in any combination.

In some embodiments, the present disclosure provides a method of treating a hyperkinetic movement disorder in a subject in need thereof, the method comprising administering to the subject a pharmaceutical composition comprising an active ingredient comprising R,R-tetrabenazine, wherein the administering provides a PK profile characterized in that (1) the plasma concentration of R,R-tetrabenazine rises during a first period of time to reach a maximum concentration for the first period of time of about 150 pg/ml to about 3500 pg/ml at a first time point, and optionally (2) after the first period of time, the plasma concentration of R,R-tetrabenazine can remain substantially constant for a sustained period of time, such as about 24 hours, about 48 hours, about 72 hours, about 96 hours, or more. In some embodiments, the first period of time refers to the initial rising portion of the PK curve until the plasma concentration starts to plateau and/or drop, whereas the sustained period of time refers to the substantially flat portion of the PK curve, see for example, the figures of this application observed from a transdermal delivery. In some embodiments, the sustained period of time can last for as long as treatment is desired. In some embodiments, the first period of time is from time 0 (the time of administration of the pharmaceutical composition) to about 24 hours thereafter, such as 0-20 hours, or 0-18 hours. In some embodiments, the first period of time can also be longer than 0-24 hours, such as 0-36 hours or 0-48 hours. In some embodiments, the first period of time is from time 0 (the time of administration of the pharmaceutical composition) to Tₘₐₓ of R,R-tetrabenazine. Typically, the plasma concentration of R,R-tetrabenazine observed during the sustained period of time ranges from about 40% to about 250% of the maximum concentration observed for the first period of time and that the plasma concentration of R,R-tetrabenazine does not vary significantly, e.g., by two-fold, during any of the 4-hour, 8-hour, and/or 12-hour intervals of the sustained period of time, e.g., the concentration at time t₁ is within about 50% to about 200% of the concentration at time t₁+12 hours, provided that both t₁ and t₁+12 hours are within the sustained period of time. In some embodiments, during the sustained period of time, the lowest plasma concentration of R,R-tetrabenazine is at least about 40%, such as at least about 50%, at least about 60%, at least about 70%, at least about 80%, at least about 90%, of the maximum concentration for the first period of time; and/or that the maximum plasma concentration of R,R-tetrabenazine is no greater than about 250%, such as no greater than about 200%, no greater than about 150%, no greater than about 120%, of the maximum concentration for the first period of time. In some embodiments, the plasma concentration of R,R-tetrabenazine at 24 hours post the first time point is about 50% to about 200%, for example, about 75% to about 150% of that at the first time point. In some embodiments, the pharmacokinetic profile in the subject is further characterized in that the administering provides a therapeutically effective plasma concentration of R,R-tetrabenazine, R,R,R-dihydrotetrabenazine (HTBZ) and S,R,R-HTBZ during the sustained period of time, and wherein during the sustained period of time, the ratio of the plasma concentration of R,R-tetrabenazine to the combined plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.) at a given time point. In some embodiments, during the sustained period of time, the ratio of plasma concentration of R,R,R-HTBZ to S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.) at a given time point. In some embodiments, during the sustained period of time, the ratio of plasma concentration of R,R-tetrabenazine:R,R,R-HTBZ:S,R,R-HTBZ can range from about 17-40:3-10:50-80 at a given time point. In some embodiments, during the sustained period of time, the ratio of the maximum plasma concentration of R,R-tetrabenazine:R,R,R-HTBZ:S,R,R-HTBZ can range from about 17-40:3-10:50-80. In some embodiments, the administering can provide a pharmacokinetic profile characterized in that the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5. In some embodiments, the PK profile is characterized in that the average terminal half-life of R,R-tetrabenazine is about 8.5 hours±40% CV. In some embodiments, the administering provides no detectable S,S-tetrabenazine, R,S,S-HTBZ, or S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism, and identifying a pharmaceutical composition that provides any of the PK profiles described in this paragraph in any combination.

The route for administering the pharmaceutical composition to achieve the PK profiles herein is not particularly limited, so long as the administering provides the pharmacokinetic profile as described hereinabove. Typically, the administering bypasses first-pass metabolism. For example, in some embodiments, the pharmaceutical composition is administered transdermally. In some embodiments, the pharmaceutical composition is administered via an injection or infusion such as an intravenous injection (not including only a bolus injection for immediate release), subcutaneous injection, or intramuscular injection. In some embodiments, the pharmaceutical composition is a depot formulation.

The administering typically continuously or substantially continuously delivers R,R-tetrabenazine to the subject to achieve the PK profiles herein. In some embodiments, the administering delivers R,R-tetrabenazine to the subject for a period of, for example, from about 8 hours to about 72 hours or more than 72 hours. For example, in some embodiments, the administrating provides a continuously or substantially continuously delivery of R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, during the period of continuous or substantially continuous delivery, the R,R-tetrabenazine can be delivered to the subject at a substantially constant rate, for example, with the average rate of delivery for each hour substantially the same, such as within 80-125% of the overall average rate. In some embodiments, during the period of continuous or substantially continuous delivery, the R,R-tetrabenazine can be delivered to the subject at a different rate, for example, with the average rate of delivery for each hour being different. For example, in some embodiments, the average rate of delivery can be high initially but then decreases over the course of continuous or substantially continuous delivery. Typically, the precise delivery rate of R,R-tetrabenazine each hour is not critical, and those skilled in the art after reviewing the present disclosure would know how to select and design a dosing regimen to deliver a desired daily dose to the subject provides a pharmacokinetic profile as described herein. For example, in some embodiments, the R,R-tetrabenazine can be delivered to the subject at a daily dose of about 0.1 mg/day to about 20 mg/day, such as about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine to provide a pharmacokinetic profile as described herein.

The hyperkinetic movement disorder recited in the methods of treatment herein include any of those described herein. Non-limiting examples include Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In some embodiments, the hyperkinetic movement disorder can be Huntington's disease, such as chorea associated with Huntington's disease. In some embodiments, the hyperkinetic movement disorder can be Wilson's disease. In some embodiments, the hyperkinetic movement disorder can be Tourette syndrome. In some embodiments, the hyperkinetic movement disorder can be restless leg syndrome. In some embodiments, the hyperkinetic movement disorder can be tardive dyskinesia. In some embodiments, the hyperkinetic movement disorder can be tic. In some embodiments, the hyperkinetic movement disorder can be dyskinetic cerebral palsy. In some embodiments, the hyperkinetic movement disorder is a dystonia or dyskinesia disorder.

The methods of treatment herein are not limited to any specific type of subjects. For example, the method herein can be administered to the subject without regard to the fed status of the subject. In some embodiments, the subject is a pediatric and adolescent patient (e.g., 6 through 18 years of age). Also, in some embodiments, the method is not limited to any specific genotyped subjects. In some embodiments, the same dose or substantially the same dose of R,R-tetrabenazine can be administered to subjects who are characterized as PM, IM, or EM. In some embodiments, the subject is characterized as EM. In some embodiments, the subject is characterized as PM. In some embodiments, the subject is characterized as IM. In some embodiments, the method does not require dose titration and/or genotype analysis, which is required when treated with either of Xenazine® and Austedo™ tablets.

The dosing regimen for the methods of treatment herein is not particularly limited, so long as the desired dose of R,R-tetrabenazine is delivered to the subject for a desired period of time at a desired rate, which includes any of those described herein. For example, in some embodiments, the pharmaceutical composition is administered to the subject in a dosing regimen suitable to achieve the PK profiles herein. In some embodiments, the pharmaceutical composition is administered to the subject once a day. In some embodiments, the pharmaceutical composition is administered to the subject once in more than a day, such as once in two days, once in three days, once in four days, once in five days, once in six days, once in a week, or once in more than a week. In some embodiments, each administration provides a continuous or substantially continuous delivery of R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. As discussed herein, there can be no lag time, overlap, or have some lag time between two consecutive administrations.

Pharmaceutical compositions suitable for the various routes of administrations of the methods herein are also described. Typically, for transdermal delivery, the pharmaceutical composition can include an adhesive composition, e.g., in a transdermal delivery device, which comprises the active ingredient dispersed in an adhesive (preferably a pressure sensitive adhesive). In some embodiments, the adhesive composition is applied to the subject to deliver about 0.1 mg/day to about 20 mg/day of R,R-tetrabenazine (e.g., any of the exemplified ranges described herein) at a substantially constant rate for up to 24 hours post application, up to 48 hours post application, up to 96 hours post application, or up to 1 week post application. Suitable adhesive composition include any of those described herein, e.g., any of those described in Embodiments 1-18 as applicable to tetrabenazine or any of the specific compositions shown in the Examples section (e.g., Example 4A). In some embodiments, the adhesive composition comprises the active ingredient dispersed in a non-reactive acrylate pressure sensitive adhesive. The active ingredient can be present in an amount of about 1% to about 20% by weight, such as about 1%, about 2%, about 3%, about 4%, about 5%, about 6%, about 7%, about 10%, about 15%, about 20%, or any ranges between the recited values, for example, about 1% to about 15%, about 2% to about 15%, about 2% to about 10%, about 2% to about 7%, about 3% to about 15%, about 3% to about 10%, about 3% to about 7%, about 5% to about 15%, about 5% to about 10%, about 5% to about 7%, about 7% to about 15%, about 7% to about 10%, about 10% to about 20%, about 10% to about 15%, etc. In some embodiments, the adhesive composition comprises a substantially pure R,R-isomer of tetrabenazine as the sole active ingredient. In some embodiments, the substantially pure R,R-isomer of tetrabenazine is in a free base form. Suitable adhesive include any of those described herein, such as any of the pressure sensitive adhesive described herein. In some embodiments, the adhesive can be a non-reactive acrylate pressure sensitive adhesive described herein, such as Duro-Tak 87-900A or described in exemplary Embodiments 2-7. The adhesive typically is present in an amount of about 50% to about 97% by weight, such as about 50%, about 60%, about 70%, about 80%, about 90%, about 95%, about 97%, or any ranges between the recited values, for example, about 50% to about 95%, about 50% to about 90%, about 50% to about 80%, about 50% to about 70%, about 50% to about 60%, about 60% to about 97%, about 60% to about 95%, about 60% to about 90%, about 60% to about 80%, about 60% to about 70%, about 70% to about 97%, about 70% to about 95%, about 70% to about 90%, about 70% to about 80%, about 80% to about 97%, about 80% to about 95%, about 80% to about 90%, etc. In some specific embodiments, the active ingredient is in an amount of about 2% to about 7% by weight and the non-reactive acrylate pressure sensitive adhesive is in an amount of about 50% to about 97% by weight. The adhesive composition can also optionally include other ingredients, such as an antioxidant, a crystallization inhibitor, a plasticizer, and/or a permeation enhancer. In some embodiments, the adhesive composition includes an antioxidant, such as a gallate antioxidant, for example, propyl gallate. The amount of antioxidant is typically included in an amount of about 0% to about 1% by weight, such as about 0.001%, about 0.01%, about 0.1%, about 0.5%, about 1%, or any ranges between the recited values, for example, about 0.001% to about 0.5%, about 0.01% to about 0.5%, etc. by weight. In some embodiments, the adhesive composition includes a crystallization inhibitor, for example, a means to prevent formation of drug crystals (crystals of the active ingredient of the adhesive composition, such as R,R-tetrabenazine) after shelf storage for two weeks at ambient temperature. In some embodiments, the adhesive composition includes a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. In some embodiments, the adhesive composition includes a crystallization inhibitor selected from a polymethacrylate (e.g., Plastoid B (copolymer of butyl methacrylate and methyl methacrylate), Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. The crystallization inhibitor is typically present in an amount of about 0 to about 40% by weight, such as about 5%, about 10%, about 15%, about 20%, about 30%, about 40%, or any range between the recited values, for example, about 10% to about 40%, about 10% to about 30%, about 10% to about 20%, 15% to about 40%, about 15% to about 30%, about 15% to about 20%, 20% to about 40%, about 20% to about 30%, etc. by weight. In some embodiments, the adhesive composition can also comprise a skin permeation enhancer as described herein, such as isopropyl myristate. In some embodiments, the adhesive composition can also be free of a skin permeation enhancer as described herein, for example, in some embodiments, the adhesive composition can also be free of isopropyl myristate. Suitable drug loadings, active surface area, thickness, adhesive properties, etc. include any of those described herein in any combinations. Suitable pharmaceutical compositions for other routes of delivery include those described herein.

### Method of Screening Pharmaceutical Compositions for Treating Hypokinetic Movement Disorder

In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism to deliver R,R-tetrabenazine to the subject continuously or substantially continuously, and identifying a pharmaceutical composition that provides any of the PK profiles described hereinabove. In some embodiments, the identifying comprises identifying a pharmaceutical composition that provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5; or the ratio of the steady state plasma concentration of R,R-tetrabenazine to the combined steady state plasma concentration of R,R,R-HTBZ and S,R,R-HTBZ ranges from about 1:1 to about 1:5. In some embodiments, the ratio of maximum plasma concentration of R,R,R-HTBZ to S,R,R-HTBZ ranges from about 1:5 to about 1:30 (e.g., about 1:10 to about 1:20). In some embodiments, the identifying comprises identifying a pharmaceutical composition that provides any of the PK profiles described herein, as applicable.

In some embodiments, the method comprises measuring the skin flux characteristics of a test adhesive composition in vitro using human cadaver skin, and identifying an adhesive composition that provides any of the in vitro flux characteristics described herein. In some embodiments, the test adhesive composition comprises about 2% to about 10% by weight (e.g., about 2% to about 7%) of R,R-tetrabenazine, the transdermal delivery device (e.g., comprising the adhesive composition with a non-reactive acrylate adhesive described herein).

The pharmaceutical composition such as adhesive composition identified herein is also novel composition of this disclosure. In some embodiments, the present disclosure also provides a method of treating a hyperkinetic movement disorder, which comprises administering the identified pharmaceutical composition to a subject in need thereof.

### Continuous Delivery of Deuterated Tetrabenazine

Some embodiments of the present disclosure are directed to continuous or substantially continuous delivery of a deuterated tetrabenazine. In any of the embodiments described herein, a deuterated tetrabenazine such as deutetrabenazine can replace, either in part or in whole, of the tetrabenazine for the respective composition, device, or methods. Those skilled in the art would understand that the in vitro and/or in vivo parameters of such replaced composition, device, or methods may differ, especially those relate to pharmacokinetics as deuteration may modify the metabolic profile of tetrabenazine.

In some embodiments, the present disclosure provides a method of treating a hyperkinetic movement disorder in a subject in need thereof, the method comprising administering to the subject a pharmaceutical composition comprising an active ingredient comprising a deuterated R,R-tetrabenazine, wherein the administering bypasses first-pass metabolism and continuously or substantially continuously delivers to the subject about 0.1 mg/day to about 20 mg/day of deuterated R,R-tetrabenazine, for example, about 0.5 mg/day to about 10 mg/day of deuterated R,R-tetrabenazine, about 0.5 mg/day to about 8 mg/day of deuterated R,R-tetrabenazine, about 0.5 mg/day to about 6 mg/day of deuterated R,R-tetrabenazine, about 0.5 mg/day to about 4 mg/day of deuterated R,R-tetrabenazine, about 1 mg/day to about 10 mg/day of deuterated R,R-tetrabenazine, about 1 mg/day to about 8 mg/day of deuterated R,R-tetrabenazine, about 1 mg/day to about 6 mg/day of deuterated R,R-tetrabenazine, about 1 mg/day to about 4 mg/day of deuterated R,R-tetrabenazine, about 2 mg/day to about 10 mg/day of deuterated R,R-tetrabenazine, about 2 mg/day to about 8 mg/day of deuterated R,R-tetrabenazine, about 2 mg/day to about 6 mg/day of deuterated R,R-tetrabenazine, about 2 mg/day to about 4 mg/day of deuterated R,R-tetrabenazine, about 4 mg/day to about 10 mg/day of deuterated R,R-tetrabenazine, about 4 mg/day to about 8 mg/day of deuterated R,R-tetrabenazine, about 4 mg/day to about 6 mg/day of deuterated R,R-tetrabenazine, about 6 mg/day to about 10 mg/day of deuterated R,R-tetrabenazine, or about 6 mg/day to about 8 mg/day of deuterated R,R-tetrabenazine. In some preferred embodiments, the method delivers about 0.5 mg/day to about 8 mg/day of deuterated R,R-tetrabenazine or about 2 mg/day to about 6 mg/day of deuterated R,R-tetrabenazine. The period of drug delivery herein can vary, for example, from about 8 hours to about 72 hours or more than 72 hours. For example, in some embodiments, the administrating provides a continuously or substantially continuously delivery of deuterated R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, during the period of continuous or substantially continuous delivery, the deuterated R,R-tetrabenazine can be delivered to the subject at a substantially constant rate, for example, with the average rate of delivery for each hour substantially the same, such as within 80-125% of the overall average rate. In some embodiments, during the period of continuous or substantially continuous delivery, the deuterated R,R-tetrabenazine can be delivered to the subject at a different rate, for example, with the average rate of delivery for each hour being different. For example, in some embodiments, the average rate of delivery can be high initially but then decreases over the course of continuous or substantially continuous delivery. Typically, the precise delivery rate of deuterated R,R-tetrabenazine each hour is not critical, and those skilled in the art after reviewing the present disclosure would know how to select and design a dosing regimen to deliver a desired daily dose to the subject, such as about 0.5 mg/day to about 10 mg/day of deuterated R,R-tetrabenazine, about 0.5 mg/day to about 8 mg/day of deuterated R,R-tetrabenazine, or about 2 mg/day to about 6 mg/day of deuterated R,R-tetrabenazine. In some embodiments, the delivery of the desired daily dose to the subject also provides a pharmacokinetic profile as described herein.

In some embodiments, the present disclosure provides a method of treating a hyperkinetic movement disorder in a subject in need thereof, the method comprising administering to the subject a pharmaceutical composition comprising an active ingredient comprising a deuterated R,R-tetrabenazine, wherein the administering provides a desired pharmacokinetic profile (PK profile). For example, in some embodiments, the desired PK profile is characterized in that the administering provides at a therapeutically effective plasma concentration of deuterated R,R-tetrabenazine, deuterated R,R,R-dihydrotetrabenazine (HTBZ) and deuterated S,R,R-HTBZ, wherein the ratio of the maximum plasma concentration of deuterated R,R-tetrabenazine to the combined maximum plasma concentration of deuterated R,R,R-HTBZ and deuterated S,R,R-HTBZ ranges from about 1:1 to about 1:7.5 (e.g., about 1:1, about 1:1.2, about 1:1.5, about 1:2, about 1:3, about 1:4, about 1:5, about 1:7.5 or any ranges between the recited values, for example, about 1:1 to about 1:3, about 1:2 to about 1:4, etc.), i.e., Cₘₐₓ of deuterated R,R-tetrabenazine/(Cₘₐₓ of deuterated R,R,R-HTBZ+Cₘₐₓ of deuterated S,R,R-HTBZ) ranges from about 1:1 to about 7.5; or the ratio of the steady state plasma concentration of deuterated R,R-tetrabenazine to the combined maximum plasma concentration of deuterated R,R,R-HTBZ and deuterated S,R,R-HTBZ ranges from about 1:1 to about 1:7.5. As used herein, a therapeutically effective plasma concentration of deuterated R,R-tetrabenazine, deuterated R,R,R-dihydrotetrabenazine (HTBZ) and deuterated S,R,R-HTBZ does not require each of the three compounds to be in a therapeutically effective plasma concentration by itself. It suffices that the three compounds in combination are therapeutically effective, for example, at its respective steady state concentrations. In some embodiments, the ratio of maximum plasma concentration or steady state plasma concentration of deuterated R,R,R-HTBZ to deuterated S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments, the ratio of maximum plasma concentration or steady state plasma concentration of deuterated R,R-tetrabenazine:deuterated R,R,R-HTBZ:deuterated S,R,R-HTBZ can range from about 14-40:3-11:50-85. In some embodiments, the administering can provide a pharmacokinetic profile characterized in that the ratio of AUC_{0-∞} of deuterated SRR-HTBZ to the AUC_{0-∞} of deuterated R,R-tetrabenazine is about 1 to about 15, such as about 1.5 to about 11, and/or the ratio of AUC_{0-∞} of deuterated RRR-HTBZ to the AUC_{0-∞} of deuterated R,R-tetrabenazine is about 0.1 to about 0.75, such as about 0.15 to about 0.5. In some embodiments, the desired PK profile can also be characterized in that the administering provides a therapeutically effective plasma concentration of deuterated R,R-tetrabenazine, deuterated R,R,R-dihydrotetrabenazine (HTBZ) and deuterated S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the desired PK profile can also be characterized in that the administering provides a substantially constant steady state plasma concentration of deuterated R,R-tetrabenazine above 150 pg/ml (e.g., about 150 pg/ml to about 3000 pg/ml), for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments, the administering provides no detectable deuterated S,S-tetrabenazine, deuterated R,S,S-HTBZ, or deuterated S,S,S-HTBZ in the plasma of the subject. In some embodiments, the method comprises administering a test pharmaceutical composition to a subject that bypasses first-pass metabolism to deliver deuterated R,R-tetrabenazine to the subject continuously or substantially continuously, and identifying a pharmaceutical composition that provides any of the PK profiles described in this paragraph in any combination.

In some embodiments, the present disclosure provides a method of treating a hyperkinetic movement disorder in a subject in need thereof, the method comprising administering to the subject a pharmaceutical composition comprising an active ingredient comprising a deuterated R,R-tetrabenazine, wherein the administering provides a PK profile characterized in that (1) the plasma concentration of deuterated R,R-tetrabenazine rises during a first period of time to reach a maximum concentration for the first period of time of about 150 pg/ml to about 3500 pg/ml at a first time point, and optionally (2) after the first period of time, the plasma concentration of deuterated R,R-tetrabenazine can remain substantially constant for a sustained period of time, such as about 24 hours, about 48 hours, about 72 hours, about 96 hours, or more. In some embodiments, the first period of time refers to the initial rising portion of the PK curve until the plasma concentration starts to plateau and/or drop, whereas the sustained period of time refers to the substantially flat portion of the PK curve, see for example, the figures of this application observed from a transdermal delivery. In some embodiments, the first period of time is from time 0 (the time of administration of the pharmaceutical composition) to about 24 hours thereafter, such as 0-20 hours, or 0-18 hours. In some embodiments, the first period of time can also be longer than 0-24 hours, such as 0-36 hours or 0-48 hours. In some embodiments, the first period of time is from time 0 (the time of administration of the pharmaceutical composition) to Tₘₐₓ of deuterated R,R-tetrabenazine. Typically, the plasma concentration of deuterated R,R-tetrabenazine observed during the sustained period of time ranges from about 40% to about 250% of the maximum concentration observed for the first period of time and that the plasma concentration of deuterated R,R-tetrabenazine does not vary significantly, e.g., by two-fold, during any of the 4-hour, 8-hour, and/or 12-hour intervals of the sustained period of time, e.g., the concentration at time t₁ is within about 50% to about 200% of the concentration at time t₁+12 hours, provided that both t₁ and t₁+12 hours are within the sustained period of time. In some embodiments, during the sustained period of time, the lowest plasma concentration of deuterated R,R-tetrabenazine is at least about 40%, such as at least about 50%, at least about 60%, at least about 70%, at least about 80%, at least about 90%, of the maximum concentration for the first period of time; and/or that the maximum plasma concentration of deuterated R,R-tetrabenazine is no greater than about 250%, such as no greater than about 200%, no greater than about 150%, no greater than about 120%, of the maximum concentration for the first period of time. In some embodiments, the plasma concentration of deuterated R,R-tetrabenazine at 24 hours post the first time point is about 50% to about 200%, for example, about 75% to about 150% of that at the first time point. In some embodiments, the pharmacokinetic profile in the subject is further characterized in that the administering provides a therapeutically effective plasma concentration of deuterated R,R-tetrabenazine, deuterated R,R,R-dihydrotetrabenazine (HTBZ) and deuterated S,R,R-HTBZ during the sustained period of time, and wherein during the sustained period of time, the ratio of the plasma concentration of deuterated R,R-tetrabenazine to the combined plasma concentration of deuterated R,R,R-HTBZ and deuterated S,R,R-HTBZ ranges from about 1:1 to about 1:7.5 (e.g., about 1:1, about 1:1.2, about 1:1.5, about 1:2, about 1:3, about 1:4, about 1:5, about 1:7.5, or any ranges between the recited values, for example, about 1:1 to about 1:3, about 1:2 to about 1:4, etc.) at a given time point. In some embodiments, during the sustained period of time, the ratio of plasma concentration of deuterated R,R,R-HTBZ to deuterated S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.) at a given time point. In some embodiments, during the sustained period of time, the ratio of plasma concentration of deuterated R,R-tetrabenazine:deuterated R,R,R-HTBZ:deuterated S,R,R-HTBZ can range from about 14-40:3-11:50-85 at a given time point. In some embodiments, during the sustained period of time, the ratio of the maximum plasma concentration of deuterated R,R-tetrabenazine:deuterated R,R,R-HTBZ:deuterated S,R,R-HTBZ can range from about 14-40:3-11:50-85. In some embodiments, the administering can provide a pharmacokinetic profile characterized in that the ratio of AUC_{0-∞} of deuterated SRR-HTBZ to the AUC_{0-∞} of deuterated R,R-tetrabenazine is about 1 to about 15, such as about 1.5 to about 11, and/or the ratio of AUC_{0-∞} of deuterated RRR-HTBZ to the AUC_{0-∞} of deuterated R,R-tetrabenazine is about 0.1 to about 0.75, such as about 0.15 to about 0.5. In some embodiments, the PK profile is characterized in that the average terminal half-life of deuterated R,R-tetrabenazine is about 8.5 hours±40% CV. In some embodiments, the administering provides no detectable deuterated S,S-tetrabenazine, deuterated R,S,S-HTBZ, or deuterated S,S,S-HTBZ in the plasma of the subject. In some embodiments, the present disclosure also provides a method of identifying a pharmaceutical composition for treating a hyperkinetic movement disorder. In some embodiments, the method comprises administering a test pharmaceutical composition to a subject that bypasses first-pass metabolism, and identifying a pharmaceutical composition that provides any of the PK profiles described in this paragraph in any combination.

The route for administering the pharmaceutical composition to achieve the PK profiles herein is not particularly limited, so long as the administering provides the pharmacokinetic profile as described hereinabove. Typically, the administering bypasses first-pass metabolism. For example, in some embodiments, the pharmaceutical composition is administered transdermally. In some embodiments, the pharmaceutical composition is administered via an injection or infusion such as an intravenous injection (not including a bolus injection), subcutaneous injection, or intramuscular injection. In some embodiments, the pharmaceutical composition is a depot formulation.

The administering typically continuously or substantially continuously delivers deuterated R,R-tetrabenazine to the subject to achieve the PK profiles herein. In some embodiments, the administering delivers deuterated R,R-tetrabenazine to the subject for a period of, for example, from about 8 hours to about 72 hours or more than 72 hours. For example, in some embodiments, the administrating provides a continuously or substantially continuously delivery of deuterated R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values. In some embodiments, during the period of continuous or substantially continuous delivery, the deuterated R,R-tetrabenazine can be delivered to the subject at a substantially constant rate, for example, with the average rate of delivery for each hour substantially the same, such as within 80-125% of the overall average rate. In some embodiments, during the period of continuous or substantially continuous delivery, the deuterated R,R-tetrabenazine can be delivered to the subject at a different rate, for example, with the average rate of delivery for each hour being different. For example, in some embodiments, the average rate of delivery can be high initially but then decreases over the course of continuous or substantially continuous delivery. Typically, the precise delivery rate of deuterated R,R-tetrabenazine each hour is not critical, and those skilled in the art after reviewing the present disclosure would know how to select and design a dosing regimen to deliver a desired daily dose to the subject provides a pharmacokinetic profile as described herein. For example, in some embodiments, the deuterated R,R-tetrabenazine can be delivered to the subject at a daily dose of about 0.1 mg/day to about 20 mg/day, such as about 0.5 mg/day to about 10 mg/day of deuterated R,R-tetrabenazine, about 0.5 mg/day to about 8 mg/day of deuterated R,R-tetrabenazine, or about 2 mg/day to about 6 mg/day of deuterated R,R-tetrabenazine to provide a pharmacokinetic profile as described herein.

The hyperkinetic movement disorder recited in the methods of treatment herein include any of those described herein. Non-limiting examples include Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In some embodiments, the hyperkinetic movement disorder can be Huntington's disease, such as chorea associated with Huntington's disease. In some embodiments, the hyperkinetic movement disorder can be Wilson's disease. In some embodiments, the hyperkinetic movement disorder can be Tourette syndrome. In some embodiments, the hyperkinetic movement disorder can be restless leg syndrome. In some embodiments, the hyperkinetic movement disorder can be tardive dyskinesia. In some embodiments, the hyperkinetic movement disorder can be tic. In some embodiments, the hyperkinetic movement disorder can be dyskinetic cerebral palsy. In some embodiments, the hyperkinetic movement disorder is a dystonia or dyskinesia disorder.

The methods of treatment herein are not limited to any specific type of subjects. For example, the method herein can be administered to the subject without regard to the fed status of the subject. In some embodiments, the subject is a pediatric and adolescent patient (e.g., 6 through 18 years of age). Also, in some embodiments, the method is not limited to any specific genotyped subjects. In some embodiments, the same dose or substantially the same dose of deuterated R,R-tetrabenazine can be administered to subjects who are characterized as PM, IM, or EM. In some embodiments, the subject is characterized as EM. In some embodiments, the subject is characterized as PM. In some embodiments, the subject is characterized as IM. In some embodiments, the method does not require dose titration and/or genotype analysis, which is required when treated with either of Xenazine® and Austedo™ tablets.

The dosing regimen for the methods of treatment herein is not particularly limited, so long as the desired dose of deuterated R,R-tetrabenazine is delivered to the subject for a desired period of time at a desired rate, which includes any of those described herein. For example, in some embodiments, the pharmaceutical composition is administered to the subject in a dosing regimen suitable to achieve the PK profiles herein. In some embodiments, the pharmaceutical composition is administered to the subject once a day. In some embodiments, the pharmaceutical composition is administered to the subject once in more than a day, such as once in two days, once in three days, once in four days, once in five days, once in six days, once in a week, or once in more than a week. In some embodiments, each administration provides a continuous or substantially continuous delivery of deuterated R,R-tetrabenazine to the subject for about 8 hours, about 12 hours, about 24 hours, about 36 hours, about 48 hours, about 72 hours, about 96 hours, about 120 hours, about 144 hours, about 168 hours, about 192 hours, or any range between the recited values.

Pharmaceutical compositions suitable for the various routes of administrations of the methods herein are also described. Typically, for transdermal delivery, the pharmaceutical composition can include an adhesive composition, e.g., in a transdermal delivery device, which comprises the active ingredient dispersed in an adhesive (preferably a pressure sensitive adhesive). In some embodiments, the adhesive composition is applied to the subject to deliver about 0.1 mg/day to about 20 mg/day of deuterated R,R-tetrabenazine (e.g., any of the exemplified ranges described herein) at a substantially constant rate for up to 24 hours post application, up to 48 hours post application, up to 96 hours post application, or up to 1 week post application. Suitable adhesive composition include any of those described herein, e.g., any of those described in Embodiments 1-18 as applicable to deuterated tetrabenazine or any of the specific compositions shown in the Examples section (e.g., Example 4A) with tetrabenazine replaced with deuterated tetrabenazine. The adhesive type, amount, the amount of active ingredient, other ingredients and amounts include any of those suitable for tetrabenazine described herein, except that the tetrabenazine replaced with deuterated tetrabenazine. In some embodiments, the adhesive composition can also comprise a skin permeation enhancer as described herein, such as isopropyl myristate. In some embodiments, the adhesive composition can also be free of a skin permeation enhancer as described herein, for example, in some embodiments, the adhesive composition can also be free of isopropyl myristate. Suitable pharmaceutical compositions for other routes of delivery include those described herein. In any of the embodiments applicable, the deuterated tetrabenazine can be a substantially pure R,R-isomer of deuterated tetrabenazine, e.g., R,R-deutetrabenazine. In some embodiments, the substantially pure R,R-isomer of deuterated tetrabenazine is the sole active ingredient in the pharmaceutical composition. In some embodiments, the substantially pure R,R-isomer of deuterated tetrabenazine is in a free base form.

While typically, the methods herein deliver either R,R-tetrabanazine or a deuterated R,R-tetrabanazine to a subject in need thereof, the present disclosure also contemplate delivering to a subject in need thereof a mixture of R,R-tetrabanazine, and a deuterated R,R-tetrabanazine.

### Transdermal Delivery of Tetrabenazine or Deuterated Tetrabenazine

As discussed herein, in various embodiments, the methods herein deliver tetrabanazine or deuterated trabenazine to a subject in need thereof transdermally. The pharmaceutical composition suitable for transdermal delivery includes any of those described herein, for example, as described in exemplary Embodiments 1-18 or any of the specific compositions shown in the Examples section (e.g., Example 4A). The transdermal pharmaceutical composition and transdermal delivery device are also novel aspects of the present disclosure.

### Transdermal Delivery Device of Tetrabenazine

In various embodiments, the present invention is directed to pharmaceutical compositions or transdermal delivery devices comprising tetrabenazine and/or a deuterated tetrabenazine (e.g., deutetrabenazine). The pharmaceutical compositions and transdermal delivery devices provide novel options for transdermally delivering tetrabenazine and/or a deuterated tetrabenazine to a subject in need thereof. Tetrabenazine and/or deuterated tetrabenazine have not been previously shown as administrable via the transdermal route. As detailed herein, the inventors have shown that the transdermal delivery device and pharmaceutical compositions disclosed herein (e.g., adhesive compositions herein) can be applied to a subject to achieve a therapeutically effective flux and therefore can be useful for the treatment of various diseases or disorders such as hyperkinetic movement disorder. See also e.g., PCT/US2019/028900, the content of which is herein incorporated by reference in its entirety.

Certain embodiments of the present disclosure are directed to a transdermal delivery device comprising tetrabenazine or a deuterated tetrabenazine (e.g., deutetrabenazine). In some embodiments, the transdermal delivery device comprises a backing layer; a drug layer comprising a drug chosen from tetrabenazine, a deuterated tetrabenazine (e.g., deutetrabenazine), and combinations thereof, and an adhesive layer defining an active surface area. In some embodiments, the drug is in an amount of about 2% to about 30% by weight of the drug layer. In some embodiments, the transdermal delivery device includes a single drug layer. In some embodiments, the transdermal delivery device includes more than one drug layers. In some embodiments, the transdermal delivery device includes a single adhesive layer. In some embodiments, the transdermal delivery device includes more than one adhesive layers.

Various patch designs can be used for the transdermal delivery device herein. For example, in some embodiments, the transdermal delivery device can be a drug-in-adhesive (DIA) patch. In some embodiments, the DIA patch is a single layer patch, wherein the drug layer and the adhesive layer are the same layer, for example, the drug is homogenously dispersed in the adhesive. In some embodiments, the DIA patch is a multilayer patch. For example, two drug-in-adhesive layers can be included in the patch, which is optionally separated by a membrane. In some embodiments, the two DIA layers can have different release characteristics, for example, one of the layers is an immediate release layer whereas the other is a controlled-release layer. In some embodiments, the two DIA layers can have different release characteristics, for example, one of the layers releases the drug relatively fast in a relatively short period of time, whereas the other layer releases the drug for a more sustained period of time.

A drug-in-reservoir (DIR) design can also be used for the transdermal delivery device herein. In some embodiments, the drug layer and the adhesive layer can be two separate layers that are laminated to each other or separated, for example, by a rate-controlling membrane. For example, in some embodiments, the drug layer is a reservoir layer, such as a drug matrix, that is laminated with the adhesive layer.

Other patch designs can also be used for the transdermal delivery device herein. For example, in some embodiments, the transdermal delivery device can be an active patch, such as an iontophoresis patch. In some embodiments, the transdermal delivery device can be a minimally invasive patch, such as a microneedle based patch. In some embodiments, the transdermal delivery device can also have another patch design that may contain chemical or physical mode of enhancement.

Typically, the transdermal delivery device (e.g., a DIA patch) is supported by an impermeable backing film, and the adhesive surface is protected by a release liner. Various materials can be used as a backing layer for the transdermal delivery device herein. Typically, the backing layer is impermeable. For example, the backing layer can be comprised of impermeable polymeric films such as polyester (PET) or polyethylene (PE) films. In some embodiments, the backing layer can comprise a polyester, such as Scotchpak 9723, Scotchpak 9736 or Scotchpak 1012, a polyurethane film, such as Scotchpak 9701, or a polyethylene film, such as CoTran 9720.

The release liner can be manufactured in the desired size for the present invention. The release liner can be comprised of silicone or fluoro-polymer coated polyester film. The release liner protects the transdermal delivery device during storage and is removed before its use. Silicone-coated release liners include those manufactured by Mylan Corporation, Loparex Corporation, and 3M's Drug Delivery Systems. The fluoro-polymer coated release liners include those manufactured and supplied by 3M's Drug Delivery Systems and Loparex. In some embodiments, the release liner comprises 3M's ScotchPak 9744 or Scotchpak 1022.

The transdermal delivery devices (e.g., DIA patches) herein can have different sizes (patch sizes) depending on its application. Typically, the patch sizes can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any ranges between the specified values), for example, about 10 cm² to about 100 cm².

When applying the transdermal delivery devices (e.g., DIA patches) herein to a skin of a subject, all of the adhesive surface can become in contact with the skin in theory. Thus, the area of the adhesive surface defines a skin contact area where the active ingredient from the device can permeate the skin, which is also referred herein to as an active surface area. In some embodiments, the adhesive surface is the only surface of the transdermal delivery device that is in contact with the skin upon application, and the active surface area is the same as the area of the adhesive surface. In some embodiments, the adhesive surface and one or more other surfaces of the transdermal delivery device are in contact with the skin upon application, and the entire skin contact area is the active surface area.

The active surface area can determine the doses of the drug to be delivered. Typically, the active surface area can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any ranges between the specified values), for example, about 10 cm² to about 100 cm².

### The Drug Layer

Typically, the drug layer comprises tetrabenazine, deuterated tetrabenazine, or a combination thereof. In some embodiments, the drug layer can optionally include one or more other ingredients, for example, selected from skin permeation enhancers, humectants, plasticizers, antioxidants, anti-irritants, gel-forming agents, drug release modifiers, solvents, crystallization inhibitors, and additional active ingredients. In some embodiments, the drug layer is adjusted such that the transdermal delivery device achieves the skin flux characteristics described herein. It should be noted that the pharmaceutical compositions used for the drug layer herein can also be a novel formulation, independent of the transdermal delivery device and/or the skin flux characteristics herein. Thus, some embodiments of the present disclosure are also directed to such pharmaceutical compositions.

In some embodiments, the drug in the drug layer can be tetrabenazine. The tetrabenazine in the transdermal delivery device described herein is not limited to a particular enantiomer and can be in a racemic form, a substantially pure R,R-tetrabenazine (e.g., with less than 10%, less than 5%, less than 1%, or less than 0.1% of the S,S-isomer), a substantially pure S,S-tetrabenazine (e.g., with less than 10%, less than 5%, less than 1%, or less than 0.1% of the R,R-isomer), or a mixture of R,R-tetrabenazine and S,S-tetrabenazine in any ratio. In some embodiments, the tetrabenazine in the transdermal delivery device is in racemic form. In some embodiments, the tetrabenazine in the transdermal delivery device is a substantially pure R,R-tetrabenazine. In some embodiments, tetrabenazine is the only drug in the drug layer. In some embodiments, tetrabenazine is the only drug in the transdermal delivery device. In some embodiments, the drug layer and/or the transdermal delivery device can also include other active ingredients.

In some embodiments, the drug in the drug layer can be a deuterated tetrabenazine. As used herein, a deuterated tetrabenazine refers to a compound resulted from substituting one or more hydrogen atoms of tetrabenazine with deuterium such that each substituted position has a deuterium content above the natural abundance, i.e., the substituted position is enriched with deuterium. In some embodiments, the deuterated tetrabenazine has at least one position with deuterium enriched to greater than 10% deuterium, greater than 50% deuterium, greater than 90% deuterium, greater than 95% deuterium or greater than 98% deuterium. A preferred example of deuterated tetrabenazine is deutetrabenazine, the racemic form of which is the active ingredient in Austedo™ tablets. The deuterated tetrabenazine in the transdermal delivery device described herein is not limited to a particular enantiomer and can be in a racemic form, a substantially pure R,R-isomer, e.g., R,R-deutetrabenazine (see below), (e.g., with less than 10%, less than 5%, less than 1%, or less than 0.1% of the S,S-isomer), a substantially pure S,S-isomer, e.g., S,S-deutetrabenazine (see below), (e.g., with less than 10%, less than 5%, less than 1%, or less than 0.1% of the R,R-isomer), or a mixture of R,R-isomer and S,S-isomer in any ratio. In some embodiments, the transdermal delivery device comprises deutetrabenazine in racemic form. In some embodiments, the transdermal delivery device comprises deutetrabenazine as a substantially pure R,R-deutetrabenazine. In some embodiments, deutetrabenazine is the only drug in the drug layer. In some embodiments, deutetrabenazine is the only drug in the transdermal delivery device. In some embodiments, the drug layer and/or the transdermal delivery device can also include other active ingredients.

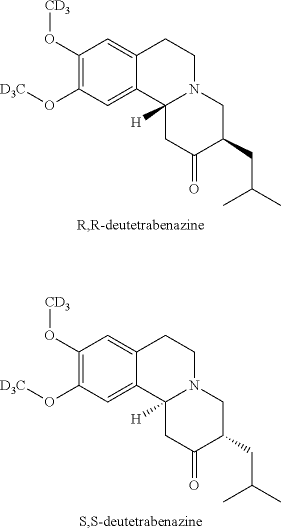 [SMILES: [2H]C([2H])([2H])Oc1cc2c(cc1OC([2H])([2H])[2H])[C@H]1CC(=O)[C@H](CC(C)C)CN1CC2 ; [2H]C([2H])([2H])Oc1cc2c(cc1OC([2H])([2H])[2H])[C@@H]1CC(=O)[C@@H](CC(C)C)CN1CC2]

The drug can be present in the drug layer of the transdermal delivery device in various amounts. In some embodiments, the drug layer comprises tetrabenazine, deuterated tetrabenazine, or a combination thereof, in an amount of about 2% to about 30% (e.g., about 2%, about 2.5%, about 5%, about 8%, about 10%, about 15%, about 18%, about 20%, about 25%, about 30%, or any ranges in between the recited values) by weight of the drug layer. In some specific embodiments, the drug layer comprises tetrabenazine, deuterated tetrabenazine, or a combination thereof, in the amount of about 2%, about 2.5%, about 5%, about 8%, about 10%, about 15%, or about 20% by weight of the drug layer. In some embodiments, tetrabenazine, deuterated tetrabenazine, or a combination thereof, only exists in the drug layer (e.g., in a drug-in-adhesive layer) of the transdermal delivery device.

The weight and thickness of the drug layer can vary depending on different factors such as drug concentration and desired duration of administration, etc. The drug layer is typically designed for application (e.g., delivering tetrabenazine or deutetrabenazine) for a period of time selected from about 8 hours, about 12 hours, about 18 hours, about 24 hours, about 2 days, about 3 days, about 4 days, about 5 days, about 6 days, and about 7 days. In some embodiments, the drug layer can have a coat weight of about 0.1 g/cm² to about 5 g/cm², e.g., about 0.05 g/cm² to about 5 g/cm², about 0.1 g/cm² to about 5 g/cm², such as about 0.05 g/cm² to about 0.90 g/cm², about 0.1 g/cm² to about 0.90 g/cm² (e.g., about 0.1 g/cm² to about 0.5 g/cm²) active surface area. In some embodiments, the drug layer can have a thickness of about 1.5 mils to about 10 mils, such as about 1.5 mils to about 3.5 mils (e.g., about 2 mils to about 3.5 mils). In some embodiments, the drug layer can have a thickness of about 0.1 mil to about 100 mil, such as about 1 mil to about 50 mil (e.g., about 1 mil to about 10 mil, or about 1.5 mil to about 3.5 mil).

Skin permeation enhancers can enhance the skin permeability of tetrabenazine or deuterated tetrabenazine through the skin and can be optionally included in the drug layer. In some embodiments, the drug layer is free or substantially free of a permeation enhancer. However, in some embodiments, various skin permeation enhancers can be included. Non-limiting useful skin permeation enhancers include, for example, sulfoxides (e.g., dimethylsulfoxide, DMSO), Azones (e.g., laurocapram), pyrrolidones (e.g., 2-pyrrolidone, 2P), alcohols and alkanols (e.g., ethanol or decanol), esters, glycols (e.g., propylene glycol (PG)), surfactants (e.g., Tween 80), terpenes, and combinations thereof. See, e.g., Williams et al., Adv Drug Deliv Rev. 27; 56(5):603-18 (2004). In some embodiments, the permeation enhancer comprises one or more compounds chosen from sulfoxides, alcohols, alkanols, esters, glycols, and surfactants. In some embodiments, the permeation enhancer comprises one or more compounds chosen from dimethyl sulfoxide (DMSO), oleic alcohol, oleayl oleate, oleic acid, levulinic acid, other fatty acids and fatty-acid esters, propylene glycol, dipropylene glycol, ethanol, and surfactants such as Tween 80. The skin permeation enhancer is typically included in the amount of about 1% to about 25% by weight of the pharmaceutical composition, for example, about 2%, about 5%, about 10%, about 15%, about 20%, about 25%, or any ranges between the specified values, by weight of the pharmaceutical composition.

Other suitable excipients useful in the preparation of transdermal delivery devices such as humectants, plasticizers, antioxidants, anti-irritants, gel-forming agents, crystallization inhibitors, drug release modifiers etc. can also be included in the drug layer (e.g., a drug-in-adhesive layer) or otherwise in the transdermal delivery device herein. In some embodiments, additional active ingredient(s) can also be included in the drug layer or otherwise in the transdermal delivery device herein. These excipients are within the knowledge of those skilled in the art, and can be found, for example, in the Handbook of Pharmaceutical Excipients, (7^{th} ed. 2012), the entire content of which is hereby incorporated by reference.

### The Adhesive Layer

The adhesive layer can be the same or a separate layer from the drug layer. In a typical DIA patch, the drug is homogeneously dispersed in an adhesive to form a drug-in-adhesive layer. Other designs, such as a DIR patch, can also include an adhesive layer separate from the drug layer, for example, by a membrane. In some embodiments, more than one adhesive layers (e.g., two or more drug-in-adhesive layers) can be used in the transdermal delivery device.

The adhesive layer typically includes a pressure sensitive adhesive (PSA). PSAs are generally known in the art. See, e.g., Tan et al., Pharm Sci & Tech Today, 2:60-69 (1999). Non-limiting useful PSAs include polyisobutylenes (PIB), silicone polymers, acrylate copolymers, and combinations thereof. In some embodiments, the pressure sensitive adhesive comprises a polyisobutylene adhesive, a silicone polymer adhesive, an acrylate copolymer adhesive, or a combination thereof. In some embodiments, the pressure sensitive adhesive comprises an acrylate copolymer adhesive. Non-limiting useful acrylate copolymers include, for example, acrylic pressure sensitive adhesives such as a poly acrylate vinyl acetate copolymer, e.g., Duro-Tak 87-2287, Duro-Tak 87-4098, Duro-Tak 87-4287, or Duro-Tak 87-2516, Duro-Tak 87-2852 or Duro-Tak 87-2194), which are manufactured by Henkel Adhesives. In some embodiments, the pressure sensitive adhesive can be a non-reactive acrylate adhesive (e.g., as described herein, such as Duro-Tak 87-900A), for example, an acrylate adhesive that has no functional groups containing reactive hydrogen moieties, or an acrylate adhesive that has no functional groups selected from epoxy, —OH, —COOH, and combinations thereof. PIBs are elastomeric polymers that are commonly used in PSAs, both as primary-base polymers and as tackifiers. PIBs are homopolymers of isobutylene and feature a regular structure of a carbon-hydrogen backbone with only terminal unsaturation. Non-limiting useful PIBs include those marketed under the trade name Oppanol by BASF. However, in some embodiments, the pressure sensitive adhesive does not contain a PIB based adhesive. The silicone polymers are a high molecular weight polydimethylsiloxane that contains residual silanol functionality (SiOH) on the ends of the polymer chains. Non-limiting useful silicone PSAs for use in pharmaceutical applications include those available from Dow Corning Corporation, for example under the trade name of BIO-PSA, e.g., BIO-7-4202. In some embodiments, the adhesive layer is about 1.5 mils to about 10 mils (e.g., about 1.5 mils to about 2 mils) thick.

One or more adhesives can be used in the adhesive layer. For example, in some embodiments, the adhesive layer can include a mixture of an acrylate copolymer adhesive (e.g., Durotak 87-2287) and a silicone adhesive (e.g., BIO-7-4202) in various ratios (e.g., a ration of acrylate adhesive to silicone adhesive ranging from about 1:10 to about 10:1). As detailed in the Examples section, varying the adhesive components can affect the flux characteristics of the transdermal delivery device.

The adhesive layer is typically formulated such that the transdermal delivery device can adhere to the skin of a user for a desired period of time. For example, in some embodiments, the transdermal delivery device is capable of adhering continuously to the skin of a user for about 8 hours, about 12 hours, about 18 hours, about 24 hours, about 2 days, about 3 days, about 4 days, about 5 days, about 6 days, or about 7 days or more.

### The Adhesive Composition

In some embodiments, the present invention also provides an adhesive composition comprising a drug chosen from tetrabenazine, deuterated tetrabenazine (e.g., deutetrabenazine), and combinations thereof in an adhesive. In some embodiments, the drug is homogenously dispersed in the adhesive. Suitable drug and adhesives include any of those described herein.

In some embodiments, the adhesive composition is capable of adhering continuously to the skin of a user for an extended period of time selected from about 8 hours, about 12 hours, about 18 hours, about 24 hours, about 2 days, about 3 days, about 4 days, about 5 days, about 6 days, or about 7 days or more.

In some embodiments, the adhesive is a pressure sensitive adhesive. In some embodiments, the pressure sensitive adhesive comprises a polyisobutylene (PIB) adhesive, a silicone polymer adhesive, an acrylate copolymer adhesive, or a combination thereof. In some embodiments, the pressure sensitive adhesive can be a non-reactive acrylate adhesive (e.g., as described herein, such as Duro-Tak 87-900A), for example, an acrylate adhesive that has no functional groups containing reactive hydrogen moieties, or an acrylate adhesive that has no functional groups selected from epoxy, —OH, —COOH, and combinations thereof. In some embodiments, the pressure sensitive adhesive does not include a polyisobutylene (PIB) adhesive and/or a silicone polymer adhesive.

The drug in the adhesive composition is preferably tetrabenazine or deutetrabenazine. In some embodiments, the drug is tetrabenazine. In some embodiments, the tetrabenazine is a substantially pure R,R-tetrabenazine. In some embodiments, the drug is deutetrabenazine. In some embodiments, the deutetrabenazine is a substantially pure R,R-deutetrabenazine. In some embodiments, tetrabenazine is the only active ingredient in the adhesive composition. In some embodiments, deutetrabenazine is the only active ingredient in the adhesive composition. In some embodiments, the tetrabenazine or deutetrabenazine is present in an amount of about 2% to about 30% (e.g., about 2%, about 2.5%, about 5%, about 8%, about 10%, about 15%, about 18%, about 20%, about 25%, about 30%, or any ranges in between the recited values) by weight of the adhesive composition. In some specific embodiments, the adhesive composition comprises tetrabenazine or deutetrabenazine in the amount of about 2%, about 2.5%, about 5%, about 8%, about 10%, about 15%, or about 20% by weight of the adhesive composition. In some embodiments, the active ingredient is present in an amount of about 2% to about 7% by weight.

In some embodiments, the adhesive composition further comprises a permeation enhancer. Suitable permeation enhancers include any of those described herein. In some embodiments, the adhesive composition is free of a permeation enhancer. In some embodiments, the adhesive composition is free of isopropyl myristate.

In some embodiments, the adhesive composition can optionally include one or more ingredients selected from humectants, plasticizers, antioxidants, anti-irritants, gel-forming agents, crystallization inhibitors, drug release modifiers, and additional active ingredients. For example, in some embodiments, the adhesive composition can comprise an antioxidant, e.g., a gallate antioxidant, such as propyl gallate. In some embodiments, the adhesive composition can comprise a crystallization inhibitor, such as a polyvinylpyrrolidone polymer, a cross-linked polyvinylpyrrolidone polymer, a polyvinylpyrrolidone copolymer, a cellulose based polymer, a polycarboxylic acid polymer, a polymethacrylate, a polyethylene glycol, polyvinyl acetate and polyvinylcaprolactame-based graft copolymer (PVAc-PVCap-PEG), or a combination thereof. In some preferred embodiments, the adhesive composition comprises a crystallization inhibitor which is a copolymer of butyl methacrylate and methyl methacrylate. In some embodiments, the adhesive composition comprises a crystallization inhibitor which is a polyethylene glycol, polyvinyl acetate and polyvinylcaprolactame-based graft copolymer.

The adhesive composition can be included in a transdermal delivery device. Typically, such transdermal delivery device also includes a backing layer and a release liner which protects the adhesive surface prior to use. In some embodiments, the adhesive composition can be included as the drug layer in any one of the transdermal delivery device described herein.

### Skin Flux Characteristics

The transdermal delivery device described herein preferably provides certain desired skin flux characteristics. Typically, the transdermal delivery device can deliver to a subject in need thereof about 0.1 mg/day/cm² to about 5 mg/day/cm² (e.g., about 0.1 mg/day/cm², about 0.2 mg/day/cm², about 0.5 mg/day/cm², about 1 mg/day/cm², about 2 mg/day/cm², about 5 mg/day/cm², or any ranges between the specified values) of the drug (e.g., tetrabenazine or deutetrabenazine), for example, over a period of time selected from about 8 hours, about 12 hours, about 18 hours, about 24 hours, about 2 days, about 3 days, about 4 days, about 5 days, about 6 days, and about 7 days. However, in some embodiments, the transdermal delivery device can deliver more than about 5 mg/day/cm², for example, about 8 mg/day/cm², about 10 mg/day/cm², about 15 mg/day/cm², about 20 mg/day/cm², or any ranges between the recited values, of the drug (e.g., tetrabenazine or deutetrabenazine). In some embodiments, the transdermal delivery device can deliver less than about 0.1 mg/day/cm², such as about 0.01 mg/day/cm², about 0.02 mg/day/cm², about 0.05 mg/day/cm², about 0.1 mg/day/cm², or any ranges between the recited values, of the drug (e.g., tetrabenazine or deutetrabenazine).

In some embodiments, the transdermal delivery device can deliver to a subject in need thereof about 0.1 mg/day/cm² to about 1 mg/day/cm² (e.g., about 0.1 mg/day/cm², about 0.2 mg/day/cm², about 0.5 mg/day/cm², about 1 mg/day/cm², or any ranges between the specified values) of the drug (e.g., tetrabenazine or deutetrabenazine), for example, over a period of time selected from about 24 hours, about 2 days, about 3 days, about 4 days, about 5 days, about 6 days, and about 7 days. In some embodiments, the transdermal delivery device can deliver about 0.1 mg/day/cm² to about 5 mg/day/cm² (e.g., about 0.1 mg/day/cm², about 0.2 mg/day/cm², about 0.5 mg/day/cm², about 1 mg/day/cm², about 5 mg/day/cm², or any ranges between the specified values) of the drug (e.g., tetrabenazine or deutetrabenazine), for example, over a period of time over a period of more than 7 days. In some embodiments, the transdermal delivery device can also deliver about 0.1 mg/day/cm² to about 5 mg/day/cm² (e.g., about 0.1 mg/day/cm², about 0.2 mg/day/cm², about 0.5 mg/day/cm², about 1 mg/day/cm², or any ranges between the specified values) of the drug (e.g., tetrabenazine or deutetrabenazine), for example, over a period of time less than 24 hours such as less than 18 hours, less than 12 hours, less than 8 hours, or less than 4 hours.

In some embodiments, the transdermal delivery device comprises tetrabenazine, and the transdermal delivery device provides one or more of the following skin flux characteristics when tested in vitro using human cadaver skin: a) a cumulative tetrabenazine permeated of about 0.1 μg/cm² to about 150 μg/cm² (e.g., about 0.1 μg/cm², about 0.5 μg/cm², about 1 μg/cm², about 5 μg/cm², about 10 μg/cm², about 15 μg/cm², about 20 μg/cm², about 50 pg/cm², about 75 μg/cm², about 100 μg/cm², about 125 μg/cm², about 150 μg/cm², or any ranges between the recited values) at 6 hours post administration based on the active surface area; b) a cumulative tetrabenazine permeated of about 2 μg/cm² to about 400 μg/cm² (e.g., about 2 μg/cm², about 5 μg/cm², about 10 μg/cm², about 15 μg/cm², about 20 μg/cm², about 50 μg/cm², about 100 μg/cm², about 200 μg/cm², about 300 μg/cm², about 400 μg/cm², or any ranges between the recited values) at 12 hours post administration based on the active surface area; and c) a cumulative tetrabenazine permeated of about 5 μg/cm² to about 1000 μg/cm² (e.g., about 5 μg/cm², about 10 μg/cm², about 15 μg/cm², about 20 μg/cm², about g/cm², about 50 μg/cm², about 100 μg/cm², about 200 μg/cm², about 300 μg/cm², about 400 μg/cm², about 600 μg/cm², about 800 μg/cm², about 1000 μg/cm², or any ranges between the recited values) at 24 hours post administration based on the active surface area. In some embodiments, the tetrabenazine is present in an amount of about 2% to about 30% (e.g., about 2% to about 20%, about 2% to about 10%, about 2% to about 5%, about 5% to about 10%, about 10% to about 15%) by weight of the drug layer. In some embodiments, the tetrabenazine is present in an amount of about 2%, about 5%, about 8%, about 10%, about 15%, about 20%, or any ranges between the recited value, by weight of the drug layer. In some embodiments, the tetrabenazine is a substantially pure R,R-tetrabenazine.

In some embodiments, the tetrabenazine is present in an amount of about 2% to about 5% by weight of the drug layer, and the transdermal delivery device provides one or more of the following skin flux characteristics when tested in vitro using human cadaver skin: a) a cumulative tetrabenazine permeated of about 0.1 μg/cm² to about 100 μg/cm² (e.g., about 0.1 μg/cm², about 0.5 μg/cm², about 1 μg/cm², about 5 μg/cm², about 10 μg/cm², about 15 pg/cm², about 20 μg/cm², about 50 μg/cm², about 75 μg/cm², about 100 μg/cm², or any ranges between the recited values) at 6 hours post administration based on the active surface area; b) a cumulative tetrabenazine permeated of about 2 μg/cm² to about 200 μg/cm² (e.g., about 2 μg/cm², about 5 μg/cm², about 10 μg/cm², about 15 μg/cm², about 20 μg/cm², about 50 μg/cm², about 100 μg/cm², about 200 μg/cm², or any ranges between the recited values) at 12 hours post administration based on the active surface area; and c) a cumulative tetrabenazine permeated of about 5 μg/cm² to about 600 μg/cm² (e.g., about 5 μg/cm², about 10 μg/cm², about 15 μg/cm², about 20 μg/cm², about 25 μg/cm², about 50 μg/cm², about 100 μg/cm², about 200 μg/cm², about 300 μg/cm², about 400 μg/cm², about 600 μg/cm², or any ranges between the recited values) at 24 hours post administration based on the active surface area.

In some embodiments, the tetrabenazine is present in an amount of about 5% to about 10% by weight of the drug layer, and the transdermal delivery device provides one or more of the following skin flux characteristics when tested in vitro using human cadaver skin: a) a cumulative tetrabenazine permeated of about 0.5 μg/cm² to about 150 μg/cm² (e.g., about 1 μg/cm², about 5 μg/cm², about 10 μg/cm², about 15 μg/cm², about 20 μg/cm², about 50 pg/cm², about 75 μg/cm², about 100 μg/cm², about 150 μg/cm², or any ranges between the recited values) at 6 hours post administration based on the active surface area; b) a cumulative tetrabenazine permeated of about 4 μg/cm² to about 400 μg/cm² (e.g., about 4 μg/cm², about 6 μg/cm², about 10 μg/cm², about 15 μg/cm², about 20 μg/cm², about 50 pg/cm², about 100 μg/cm², about 200 μg/cm², about 400 μg/cm², or any ranges between the recited values) at 12 hours post administration based on the active surface area; and c) a cumulative tetrabenazine permeated of about 6 μg/cm² to about 1000 μg/cm² (e.g., about 6 μg/cm², about 10 μg/cm², about 15 μg/cm², about 20 μg/cm², about 25 μg/cm², about 50 pg/cm², about 100 μg/cm², about 200 μg/cm², about 300 μg/cm², about 400 μg/cm², about 600 μg/cm², about 1000 μg/cm², or any ranges between the recited values) at 24 hours post administration based on the active surface area.

In some embodiments, the tetrabenazine is present in an amount of about 10% to about 15% by weight of the drug layer, and the transdermal delivery device provides one or more of the following skin flux characteristics when tested in vitro using human cadaver skin: a) a cumulative tetrabenazine permeated of about 0.5 μg/cm² to about 150 μg/cm² (e.g., about 1 μg/cm², about 2 μg/cm², about 5 μg/cm², about 10 μg/cm², about 15 μg/cm², about 20 pg/cm², about 50 μg/cm², about 75 μg/cm², about 100 μg/cm², about 150 μg/cm², or any ranges between the recited values) at 6 hours post administration based on the active surface area; b) a cumulative tetrabenazine permeated of about 4 μg/cm² to about 400 μg/cm² (e.g., about 4 μg/cm², about 6 μg/cm², about 10 μg/cm², about 15 μg/cm², about 20 μg/cm², about g/cm², about 40 μg/cm², about 50 μg/cm², about 100 μg/cm², about 200 μg/cm², about 400 μg/cm², or any ranges between the recited values) at 12 hours post administration based on the active surface area; and c) a cumulative tetrabenazine permeated of about 8 μg/cm² to about 1000 μg/cm² (e.g., about 8 μg/cm², about 10 μg/cm², about 15 μg/cm², about 20 pg/cm², about 30 μg/cm², about 40 μg/cm², about 50 μg/cm², about 60 μg/cm², about 100 pg/cm², about 200 μg/cm², about 300 μg/cm², about 400 μg/cm², about 600 μg/cm², about 1000 μg/cm², or any ranges between the recited values) at 24 hours post administration based on the active surface area.

In some embodiments, the transdermal delivery device comprises a deuterated tetrabenazine (e.g., deutetrabenazine), and the transdermal delivery device provides one or more of the following skin flux characteristics when tested in vitro using human cadaver skin: a) a cumulative deuterated tetrabenazine permeated of about 0.1 μg/cm² to about 150 μg/cm² (e.g., about 0.1 μg/cm², about 0.5 μg/cm², about 1 μg/cm², about 5 μg/cm², about g/cm², about 15 μg/cm², about 20 μg/cm², about 50 μg/cm², about 75 μg/cm², about 100 μg/cm², about 125 μg/cm², about 150 μg/cm², or any ranges between the recited values) at 6 hours post administration based on the active surface area; b) a cumulative deuterated tetrabenazine permeated of about 2 μg/cm² to about 400 μg/cm² (e.g., about 2 μg/cm², about 5 μg/cm², about 10 μg/cm², about 15 μg/cm², about 20 μg/cm², about 50 μg/cm², about 100 μg/cm², about 200 μg/cm², about 300 μg/cm², about 400 μg/cm², or any ranges between the recited values) at 12 hours post administration based on the active surface area; and c) a cumulative deuterated tetrabenazine permeated of about 5 μg/cm² to about 1000 μg/cm² (e.g., about 5 μg/cm², about 10 μg/cm², about 15 μg/cm², about 20 μg/cm², about g/cm², about 50 μg/cm², about 100 μg/cm², about 200 μg/cm², about 300 μg/cm², about 400 μg/cm², about 600 μg/cm², about 800 μg/cm², about 1000 μg/cm², or any ranges between the recited values) at 24 hours post administration based on the active surface area. In some embodiments, the deuterated tetrabenazine is present in an amount of about 2% to about 30% (e.g., about 2% to about 20%, about 2% to about 10%, about 2% to about 5%, about 5% to about 10%, about 10% to about 15%) by weight of the drug layer. In some embodiments, the deuterated tetrabenazine is present in an amount of about 2%, about 5%, about 8%, about 10%, about 15%, about 20%, or any ranges between the recited value, by weight of the drug layer. In some embodiments, the deuterated tetrabenazine is a substantially pure R,R-deutetrabenazine.

Transdermal delivery devices with the above flux characteristics can be prepared by those skilled in the art in view of the present disclosure. Preparations of a few transdermal delivery devices are also exemplified in the Examples section. The cumulative drug (tetrabenazine, deuterated tetrabenazine, or a combination thereof) permeated can be adjusted, for example, by varying the composition of the drug layer (e.g., drug concentration, permeation enhancer, coat weight, types of adhesives etc.).

### Compositions Containing Non-Reactive Adhesives

Compositions using non-reactive adhesives can provide certain advantages. As illustrated in the Examples section, tetrabenazine formulations with non-reactive adhesives can be more stable compared to corresponding formulations with adhesives having functional groups. For example, when a non-reactive adhesive DuroTak 87-900A was used as a matrix polymer, which is understood to be a copolymer of 2-EHA (2-ethyl hexyl acrylate) (about 45 wt % based on monomer composition), MA (methyl acrylate) (about 35 wt % based on monomer composition) and t-OA (tert octyl acrylamide) (about 20 wt % based on monomer composition), the resulted tetrabenazine adhesive composition was found to be stable after shelf storage for 4 weeks at 40° C., with no drug crystals observed and no drug degradations. In contrast, tetrabenazine adhesive composition formed using a more common adhesive matrix polymer, DuroTak 87-2287 (containing epoxy and hydroxyl functional groups), or Duro-Tak 87-2677 (containing carboxylic acid functional groups), the resulted formulation showed yellowish color after shelf storage for 4 weeks at 40° C., indicating instability of the active ingredient due to oxidation and/or other degradation.

In some embodiments, the present invention provides an adhesive composition comprising an active ingredient (or alternatively referred to as “drug”) dispersed (e.g., homogenously dispersed or dissolved) in a non-reactive acrylate pressure sensitive adhesive, wherein the active ingredient is selected from tetrabenazine, deuterated tetrabenazine, or a combination thereof. Unless otherwise obvious from context, in any of the embodiments described herein, the active ingredient can exist predominantly (e.g., at least 80%, at least 90%, or at least 95% by weight) in its free base form, for example, as tetrabenazine base, deutetrabenazine base, etc. The non-reactive acrylate pressure sensitive adhesive is typically present in an amount of about 50% to about 97% (e.g., about 50%, about 60%, about 65%, about 70%, about 75%, about 80%, about 85%, about 90%, about 95%, about 97%, or any ranges between the recited values) by weight of the adhesive composition.

Non-reactive acrylate pressure sensitive adhesives useful for embodiments of the present disclosure include those sold by Henkel, for example, under the product name DuroTak 87-900A. U.S. Pat. No. 9,056,060, the contents of which is herein incorporated by reference in its entirety, also describes non-reactive acrylate pressure sensitive adhesives, which can be used for embodiments of the present disclosure.

In some embodiments, the non-reactive acrylate pressure sensitive adhesive does not have functional groups containing reactive hydrogen moieties. In some embodiments, the non-reactive acrylate pressure sensitive adhesive does not have functional groups selected from epoxy, —OH, —COOH, and combinations thereof. For example, in some embodiments, the non-reactive acrylate pressure sensitive adhesive is a copolymer of alkyl acrylate without functional groups containing reactive hydrogen moieties or without functional groups selected from epoxy, —OH, —COOH, and combinations thereof. In some embodiments, the non-reactive acrylate pressure sensitive adhesive can be a copolymer of monomers comprising, consisting essentially of, or consisting of alkyl acrylates. For example, in some embodiments, the non-reactive acrylate pressure sensitive adhesive can be a copolymer derived from monomers consisting of alkyl acrylates, e.g., a copolymer derived from a monomer of C₂-C₁₈ alkyl acrylate (preferably C₄-C₁₀ branched or straight-chain alkyl acrylate) and a monomer of methyl acrylate, more preferably, a copolymer derived from a monomer of hexylethyl acrylate (e.g., 2-ethyl hexyl acrylate) and a monomer of methyl acrylate. In some embodiments, the copolymer of alkyl acrylate is a copolymer of hexylethyl acrylate (e.g., 2-ethyl hexyl acrylate) and methyl acrylate, and optionally other monomer(s) with no functional groups containing reactive hydrogen moieties, such as —OH, —COOH groups. In some embodiments, the non-reactive acrylate pressure sensitive adhesive can be a copolymer derived from monomers including alkyl acrylates and other monomers with no functional groups containing reactive hydrogen moieties, such as —OH, —COOH groups. For example, in some embodiments, the non-reactive acrylate pressure sensitive adhesive can be a copolymer derived from a monomer of hexylethyl acrylate (e.g., 2-ethyl hexyl acrylate), a monomer of methyl acrylate, and one or more monomers with no functional groups containing reactive hydrogen moieties, such as —OH, —COOH groups, such as acrylamide monomers (e.g., tert octyl acrylamide, dimethyl acrylamide, isopropyl acrylamide, or vinyl acetamide). As used herein, amide NH or amide NH₂ groups should not be considered as reactive hydrogen moieties. In some embodiments, the non-reactive acrylate pressure sensitive adhesive can be a copolymer derived from a monomer of C₂-C₁₈ alkyl acrylate (preferably C₄-C₁₀ branched or straight-chain alkyl acrylate), a monomer of methyl acrylate, and one or more acrylamide monomers (e.g., tert octyl acrylamide) with no functional groups selected from epoxy, —OH, —COOH groups, and combinations thereof. The weight percentages of the monomers can vary, for example, in some embodiments, the percentage of the monomers of the non-reactive acrylate pressure sensitive adhesive can be the following: the percentage of the monomer of C₂-C₁₈ alkyl acrylate (preferably C₄-C₁₀ branched or straight-chain alkyl acrylate, such as 2-ethylhexyl acrylate) can be at about 45 wt %, the percentage of the monomer of methyl acrylate can be at about 35 wt %, and the percentage of the monomer of the one or more acrylamide monomers (e.g., tert octyl acrylamide) can be at about 20 wt %.

The non-reactive acrylate pressure sensitive adhesive typically does not include vinyl acetate. The non-reactive acrylate pressure sensitive adhesive typically also does not include a crosslinker. The non-reactive acrylate pressure sensitive adhesive typically can have various viscosities. In some embodiments, the non-reactive acrylate pressure sensitive adhesive can have a viscosity of about 1,500 cP to about 20,000 cP, more preferably, about 1,500 cP to about 10,000 cP, such as about 1,800 cP, about 5,000 cP, about 10,000 cP, or ranges between the recited values. In some embodiments, the non-reactive acrylate pressure sensitive adhesive can be selected such that adhesive composition can adhere continuously to the skin of a user for about 8 hours, about 12 hours, about 18 hours, about 24 hours, about 2 days, about 3 days, about 4 days, about 5 days, about 6 days, or about 7 days or more.

In some formulations, addition of a permeation enhance can cause significant degradations of the active ingredient (e.g., tetrabenazine) in the patch formulation. Thus, in some embodiments, the adhesive composition can be free or substantially free of a permeation enhancer. For example, in some embodiments, the adhesive composition herein can be free of a permeation enhancer. In some embodiments, the adhesive composition herein can be free of a permeation enhance selected from fatty alcohols, fatty acids, fatty esters and combinations thereof. In some embodiments, the adhesive composition herein can be free of isopropyl myristate. However, it should be understood that in some cases, permeation enhancers can be added to the adhesive compositions, for example, in an amount that does not cause significant degradation of the active ingredient.

Antioxidants are typically included in the adhesive composition herein. For example, antioxidants can be added to reduce the extent of degradation of the active ingredient. However, unexpectedly, it was found that some antioxidants work better than others in protecting tetrabenazine from degradation. For example, adhesive compositions containing a gallate antioxidant (e.g., propyl gallate in the examples) were found to be shelf stable. In any of the embodiments herein, shelf stable, storage stable, or stable after shelf storage, and the like, can mean that a tested device or composition, after shelf storage at 40° C. for 4 weeks, (1) HPLC analysis shows that the tested device or composition is free or substantially free (e.g., less than 1%, less than 0.5%, less than 0.05%, or not detected by HPLC) of one or more (preferably all) degradants selected from TBZ01, TBZ02, and TBZ04; and/or (2) no drug crystals are observed (e.g., visually observed). In some embodiments, all of the degradants TBZ01, TBZ02, and TBZ04 are not detected by HPLC or below the limit for quantification in a shelf stable device or composition herein after shelf storage at 40° C. for 4 weeks. Exemplary conditions for HPLC analysis and retention times of TBZ01, TBZ02, and TBZ04 are shown in Example 5. However, when no antioxidant is used or the added antioxidant is BHT, degradants (including TBZ01, TBZ02, and TBZ04) were formed. In some embodiments, the adhesive composition can comprise a gallate antioxidant. In some preferred embodiments, the adhesive composition can comprise propyl gallate. In some preferred embodiments, propyl gallate is the only antioxidant in the adhesive composition. In some embodiments, other antioxidants can be used in combination with the propyl gallate. In some embodiments, the adhesive composition can also include propyl gallate, citric acid, ascorbic acid, vitamin E or tocopherol acetate, or a combination thereof, as an antioxidant. When present, the antioxidant, such as propyl gallate, is typically present in an amount of about 0.001% to about 0.5% (e.g., about 0.01%, about 0.02%, about 0.03%, about 0.04%, about 0.05%) by weight of the adhesive composition.

The active ingredient is typically present in the adhesive composition in an amount of about 2% to about 30% by weight. Preferably, in some embodiments, the active ingredient can be present in the adhesive composition in an amount of about 2% to about 15% by weight, such as about 2% to about 10%, or about 2% to about 7% by weight. The tetrabenazine in the adhesive compositions herein is not limited to a particular enantiomer and can be in a racemic form, a substantially pure R,R-tetrabenazine (e.g., with less than 10%, less than 5%, less than 1%, or less than 0.1% of the S,S-isomer), a substantially pure S,S-tetrabenazine (e.g., with less than 10%, less than 5%, less than 1%, or less than 0.1% of the R,R-isomer), or a mixture of R,R-tetrabenazine and S,S-tetrabenazine in any ratio. Similarly, the deuterated tetrabenazine in the adhesive compositions described herein is not limited to a particular enantiomer and can be in a racemic form, a substantially pure R,R-isomer, e.g., R,R-deutetrabenazine, (e.g., with less than 10%, less than 5%, less than 1%, or less than 0.1% of the S,S-isomer), a substantially pure S,S-isomer, e.g., S,S-deutetrabenazine, (e.g., with less than 10%, less than 5%, less than 1%, or less than 0.1% of the R,R-isomer), or a mixture of R,R-isomer and S,S-isomer in any ratio. In some embodiments, the only active ingredient in the adhesive composition is tetrabenazine, such as a substantially pure R,R-isomer of tetrabenazine. In some embodiments, the only active ingredient in the adhesive composition is deutetrabenazine, such as a substantially pure R,R-isomer of deutetrabenazine. In some embodiments, the adhesive compositions can also include other active ingredients, e.g., as described herein.

Typically, a crystallization inhibitor is also included in the adhesive composition to prevent formation of drug crystals upon storage. Such drug crystals could retard skin permeation of such adhesive compositions. Accordingly, in some embodiments, the adhesive composition can include a crystallization inhibitor in an amount effective to prevent formation of drug crystals after shelf storage for two weeks at ambient temperature. In some embodiments, the adhesive composition can include a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. In some embodiments, the crystallization inhibitor is not Kollidon VA64 (BASF).

In some preferred embodiments, the adhesive composition can include a crystallization inhibitor selected from a polymethacrylate (e.g., Plastoid B (copolymer of butyl methacrylate and methyl methacrylate), Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. For example, in some specific embodiments, the adhesive composition includes a copolymer of butyl methacrylate and methyl methacrylate, such as a polymer under tradename the Plastoid B, manufactured by Evonic. In some specific embodiments, the adhesive composition includes a polyethylene glycol, polyvinyl acetate and polyvinylcaprolactame-based graft copolymer, such as a polymer under the tradename Soluplus, manufactured by BASF. The crystallization inhibitor, when present in the adhesive composition, typically is in an amount of about 5% to about 40% by weight, such as about 10%, about 20%, about 30%, about 40%, by weight, or any range between the recited value.

In some specific embodiments, the present invention provides adhesive compositions with the following ingredients:

| | Percentage by weight (dry) | | |
|---|---|---|---|
| Ingredient | Typical | Preferred | More preferred |
| Active ingredient | 2-10% | 5-10% | 5-7% (e.g., |
| (e.g., tetrabenazine base) | | | 6.8%, 7.1%) |
| Adhesive (PSA) | 50-97% | 60-95% | 65-95% (e.g., |
| (e.g., Duro-Tak 87- | | | 72.9%, 92.5%) |
| 900A) | | | |
| Antioxidants | 0-1% | 0.001-0.5% | 0.01-0.1% (e.g., |
| (e.g., propyl gallate) | | | 0.05%) |
| Crystallization Inhibitors | 0-40% | 10-40% | 15-30% (e.g., |
| (e.g., Plastoid B, | | | 20%, 19.3%) |
| Soluplus) | | | |

The numeric values in the table should be understood as preceded by the term “about.” Other ingredients can be optionally included. In some embodiments, the adhesive composition is free of a permeation enhancer. In some embodiments, the adhesive composition is free of isopropyl myristate. Suitable active ingredients, adhesives (e.g., non-reactive acrylate adhesive), antioxidants, and crystallization inhibitors include those described herein. For example, in some preferred embodiments, the active ingredient is tetrabenazine base (e.g., a substantially pure R,R-isomer), the adhesive is an acrylate polymer, such as non-reactive acrylate adhesive, preferably, a copolymer of hexylethyl acrylate and methyl acrylate, such as Duro-Tak 87-900A, antioxidant is preferably propyl gallate, and crystallization inhibitor is preferably a copolymer of butyl methacrylate and methyl methacrylate or a polyethylene glycol, polyvinyl acetate and polyvinylcaprolactame-based graft copolymer. In some embodiments, the present disclosure also provides a method of preparing an adhesive composition (e.g., described herein). In some embodiments, the method comprising mixing an active ingredient (e.g., tetrabenazine base), an adhesive, optionally an antioxidant, optionally a crystallization inhibitor, in a suitable solvent (e.g., ethanol, etc.) to form a wet adhesive composition. Suitable amounts and suitable active ingredients, adhesives, antioxidants, crystallization inhibitors include any of those described and preferred herein, e.g., discussed in this paragraph and the preceding 10 paragraphs. Other optional ingredients and amounts thereof are also described herein. In some embodiments, the method further comprising casting the wet adhesive composition onto a backing layer. In some embodiments, the method further comprising drying the wet adhesive composition. The products produced by the methods herein are also novel compositions.

The adhesive composition (e.g., the adhesive composition with a non-reactive acrylate adhesive described herein) is typically included in a transdermal delivery device, for example, as a drug layer or drug-in-adhesive layer. For example, in some embodiments, the present disclosure provides a transdermal delivery device, which includes a backing layer, any of the adhesive compositions described herein (e.g., the adhesive composition with a non-reactive acrylate adhesive described herein as a drug layer or drug-in-adhesive layer), and a release liner. The transdermal delivery device can be cut into different sizes as desired, which is typically about 10 cm² to about 100 cm². Other patch designs are described herein. Preferably, the transdermal delivery device (and/or the adhesive composition, e.g., with a non-reactive acrylate adhesive described herein) herein is storage stable (or alternatively referred to as shelf stable), for example, with no drug crystals observed after shelf storage at 40° C. for 4 weeks, and/or with no drug degradation observed by HPLC after shelf storage at 40° C. for 4 weeks. For example, in some embodiments, the transdermal delivery device (and/or the adhesive composition e.g., with a non-reactive acrylate adhesive described herein) herein can be storage stable for 4 weeks or more, 8 weeks or more, 12 weeks or more, 16 weeks or more, 6 months or more, 12 months or more, etc.

Typically, the transdermal delivery device (and/or the adhesive composition) herein can also be configured to achieve a desired skin permeability of the active ingredient (e.g., tetrabenazine or deutetrabenazine). For example, in some embodiments, the transdermal delivery device (e.g., comprising the adhesive composition with a non-reactive acrylate adhesive described herein) can deliver to a subject in need thereof about 0.01 mg/day/cm² to about 5 mg/day/cm² (e.g., about 0.01 mg/day/cm², about 0.02 mg/day/cm², about 0.05 mg/day/cm², about 0.1 mg/day/cm², about 0.2 mg/day/cm², about 0.5 mg/day/cm², about 1 mg/day/cm², about 2 mg/day/cm², about 5 mg/day/cm², or any ranges between the specified values) of the active ingredient (e.g., tetrabenazine or deutetrabenazine), for example, over a period of time selected from about 8 hours, about 12 hours, about 18 hours, about 24 hours, about 2 days, about 3 days, about 4 days, about 5 days, about 6 days, and about 7 days. In some embodiments, the transdermal delivery device (e.g., comprising the adhesive composition with a non-reactive acrylate adhesive described herein) can deliver more than about 5 mg/day/cm², for example, about 8 mg/day/cm², about 10 mg/day/cm², about 15 mg/day/cm², about 20 mg/day/cm², or any ranges between the recited values, of the drug (e.g., tetrabenazine or deutetrabenazine). In some embodiments, the transdermal delivery device (e.g., comprising the adhesive composition with a non-reactive acrylate adhesive described herein) can deliver less than about 0.1 mg/day/cm², such as about 0.01 mg/day/cm², about 0.02 mg/day/cm², about 0.05 mg/day/cm², about 0.1 mg/day/cm², or any ranges between the recited values, of the active ingredient (e.g., tetrabenazine or deutetrabenazine).

For example, in some embodiments, the adhesive composition of the transdermal delivery device comprises about 2% to about 10% by weight (e.g., about 2% to about 7%) of tetrabenazine, the transdermal delivery device (e.g., comprising the adhesive composition with a non-reactive acrylate adhesive described herein) provides one or more of the following skin flux characteristics when tested in vitro using human cadaver skin: a) a cumulative tetrabenazine permeated of about 0.5 μg/cm² to about 50 μg/cm² (e.g., about 1 μg/cm², about 5 μg/cm², about 10 μg/cm², about 15 μg/cm², about 20 μg/cm², about 50 μg/cm², or any ranges between the recited values) at 8 hours post administration based on the active surface area; b) a cumulative tetrabenazine permeated of about 5 μg/cm² to about 500 μg/cm² (e.g., about 5 μg/cm², about 10 μg/cm², about 15 μg/cm², about 20 μg/cm², about 50 μg/cm², about 100 μg/cm², about 200 μg/cm², about 500 μg/cm², or any ranges between the recited values) at 24 hours post administration based on the active surface area; and c) a cumulative tetrabenazine permeated of about 10 μg/cm² to about 1000 μg/cm² (e.g., about 10 μg/cm², about 20 μg/cm², about 50 μg/cm², about 100 μg/cm², about 200 pg/cm², about 300 μg/cm², about 400 μg/cm², about 600 μg/cm², about 1000 μg/cm², or any ranges between the recited values) at 48 hours post administration based on the active surface area. In some embodiments, the in vitro test is conducted in accordance with the method described in Example 6 of this application.

### Methods of Administering Tetrabenazine

In various embodiments, the present invention further provides a method of using the transdermal delivery device or pharmaceutical compositions (e.g., adhesive compositions herein) described herein. In some embodiments, the transdermal delivery devices or pharmaceutical compositions can be used for any indication for which inhibition of VMAT-2 is beneficial. In some embodiments, the transdermal delivery devices or pharmaceutical compositions can be used for treating or preventing a disease or disorder mediated by VMAT-2. In some embodiments, the transdermal delivery devices or pharmaceutical compositions can be used for any indication for which administering tetrabenazine or deuterated tetrabanazine is beneficial. For example, in addition to the indication of chorea associated with Huntington's disease, other indications approved for use or associated with tetrabenazine or deuterated tetrabenazine include hemiballismus, tic disorders, tardive dyskinesia, and Tourette's syndrome. And in various embodiments, the transdermal delivery devices or pharmaceutical compositions can also be used for any of these indications.

In some embodiments, the present invention provides a method of administering tetrabenazine, deuterated tetrabenazine, or a combination thereof to a subject (e.g., human subject) in need thereof. In some embodiments, the method comprises applying any of the transdermal delivery devices or pharmaceutical compositions (e.g., adhesive compositions herein) to the subject, for example, to the skin of the subject. In some embodiments, the subject (e.g., human subject) is characterized as having a hyperkinetic movement disorder (e.g., a chronic hyperkinetic movement disorder). In some embodiments, the hyperkinetic movement disorder is selected from chorea associated with Huntington's disease, Wilson's disease, Tourette syndrome, restless leg syndrome, tardive dyskinesia, tic, and combinations thereof. In some embodiments, the method comprises applying a transdermal delivery device comprising tetrabenazine (e.g., a substantially pure R,R-tetrabenazine). In some embodiments, the method comprises applying a transdermal delivery device comprising deutetrabenazine (e.g., a substantially pure R,R-deutetrabenazine).

In some embodiments, the present invention also provides a method of inhibiting VMAT-2 in a subject in need thereof. In some embodiments, the method comprises applying any of the transdermal delivery devices or pharmaceutical compositions (e.g., adhesive compositions herein) to the subject, for example, to the skin of the subject.

In some embodiments, the present invention also provides a method of treating a vesicular monoamine transporter isoform 2 (VMAT2) mediated disease or disorder in a subject (e.g., a human subject) in need thereof. In some embodiments, the method comprises applying any of the transdermal delivery devices or pharmaceutical compositions (e.g., adhesive compositions herein) to the subject, for example, to the skin of the subject. VMAT2-mediated diseases or disorders, include, but are not limited to, hyperkinetic movement disorders (e.g., chronic hyperkinetic movement disorders), Huntington's disease, hemiballismus, senile chorea, tic disorders, tardive dyskinesia, dystonia, Tourette's syndrome, depression, cancer, rheumatoid arthritis, psychosis, multiple sclerosis, asthma, and/or any disorder which can lessened, alleviated, or prevented by administering a VMAT2 inhibitor. In some embodiments, the VMAT2-mediated disease or disorder is tardive dyskinesia. In some embodiments, the VMAT2-mediated disease or disorder is Huntington's disease. In some embodiments, the VMAT2-mediated disease or disorder is hemiballismus. In some embodiments, the VMAT2-mediated disease or disorder is Tourette's syndrome.

In some specific embodiments, the present invention provides a method of treating a hyperkinetic movement disorder in a subject (e.g., human subject) in need thereof. In some embodiments, the method comprises applying any of the transdermal delivery devices or pharmaceutical compositions (e.g., adhesive compositions herein) to the subject, for example, to the skin of the subject. In some embodiments, the hyperkinetic movement disorder is a chronic hyperkinetic movement disorder. In some embodiments, the hyperkinetic movement disorder is chorea associated with Huntington's disease, Wilson's disease, Tourette syndrome, restless leg syndrome, tardive dyskinesia, and/or a tic. In some embodiments, the hyperkinetic movement disorder is chorea associated with Huntington's disease. In some specific embodiments, the present invention provides a method of treating chorea associated with Huntington's disease in a subject in need thereof, the method comprising applying any of the transdermal delivery devices or pharmaceutical compositions (e.g., adhesive compositions herein) to the subject, for example, to the skin of the subject. In some specific embodiments, the present invention provides a method of treating tardive dyskinesia in a subject in need thereof, the method comprising applying any of the transdermal delivery devices or pharmaceutical compositions (e.g., adhesive compositions herein) to the subject, for example, to the skin of the subject. In some specific embodiments, the present invention provides a method of treating Tourette syndrome in a subject in need thereof, the method comprising applying any of the transdermal delivery devices or pharmaceutical compositions (e.g., adhesive compositions herein) to the subject, for example, to the skin of the subject. In some specific embodiments, the present invention provides a method of treating a tic in a subject in need thereof, the method comprising applying any of the transdermal delivery devices or pharmaceutical compositions (e.g., adhesive compositions herein) to the subject, for example, to the skin of the subject. In some specific embodiments, the present invention provides a method of treating hemiballismus in a subject in need thereof, the method comprising applying any of the transdermal delivery devices or pharmaceutical compositions (e.g., adhesive compositions herein) to the subject, for example, to the skin of the subject.

Tetrabenazine and/or a deuterated tetrabenazine (e.g., deutetrabenazine) can be used for the methods herein. Typically, the method comprises applying a transdermal delivery device herein which includes either tetrabenazine or deutetrabenazine as the only active ingredient. In some embodiments, the method comprises applying a transdermal delivery device herein which comprises a substantially pure R,R-tetrabenazine, e.g., with the substantially pure R,R-tetrabenazine as the only active ingredient. In some embodiments, the method comprises applying a transdermal delivery device herein which comprises a substantially pure R,R-deutetrabenazine e.g., with the substantially pure R,R-deutetrabenazine as the only active ingredient.

In any of the embodiments described herein, the method can comprise administering to the subject about 0.1 mg/day/cm² to about 5 mg/day/cm² (e.g., about 0.1 mg/day/cm², about 0.2 mg/day/cm², about 0.5 mg/day/cm², about 1 mg/day/cm², about 2 mg/day/cm², about 5 mg/day/cm², or any ranges between the specified values) of the drug (e.g., tetrabenazine or deutetrabenazine), for example, over a period of time selected from about 8 hours, about 12 hours, about 18 hours, about 24 hours, about 2 days, about 3 days, about 4 days, about 5 days, about 6 days, and about 7 days. However, in some embodiments, the method can comprise administering to the subject more than about 5 mg/day/cm², for example, about 8 mg/day/cm², about 10 mg/day/cm², about 15 mg/day/cm², about 20 mg/day/cm², or any ranges between the recited values, of the drug (e.g., tetrabenazine or deutetrabenazine). In some embodiments, the method can also comprise administering to the subject less than about 0.1 mg/day/cm², such as about 0.01 mg/day/cm², about 0.02 mg/day/cm², about 0.05 mg/day/cm², about 0.1 mg/day/cm², or any ranges between the recited values, of the drug (e.g., tetrabenazine or deutetrabenazine).

In some embodiments, the present invention provides a method of treating a vesicular monoamine transporter isoform 2 (VMAT2) mediated disease or disorder in a subject (e.g., a human subject) in need thereof, comprising transdermally administering to the subject a therapeutically effective amount of tetrabenazine or deutetrabenazine. Suitable VMAT2 mediated diseases or disorders are described herein.

Various advantages are associated with the methods described herein. For example, in some embodiments, the method can decrease inter-individual variation in plasma levels of tetrabenazine or deutetrabenazine or a metabolite thereof compared to equal doses of oral administration of tetrabenazine or deutetrabenazine. In some embodiments, the method can decrease Cₘₐₓ (e.g., by 10%, 40%, 60%, or more) of tetrabenazine or deutetrabenazine or a metabolite thereof compared to equal doses of oral administration of tetrabenazine or deutetrabenazine, for example, without also reducing therapeutic efficacy. In some embodiments, the method can provide similar plasma levels of tetrabenazine or deutetrabenazine or a metabolite thereof when equal doses are administered to subjects who are genotyped based on CYP2D6 expression as poor metabolizer (PM), intermediate metabolizer (IM), or extensive metabolizer (EM). In some embodiments, the same dose or substantially the same dose of tetrabenazine or deutetrabenazine can be administered to subjects who are characterized as PM, IM, or EM. In some embodiments, the methods herein can transdermally administer the drug (e.g., tetrabenazine or deutetrabenazine) to the subject without regard to whether the subject is characterized as PM, IM, or EM based on CYP2D6 expression.

### Exemplary Transdermal Delivery of Tetrabenazine or Deuterated Tetrabenazine

In some embodiments, the tetrabenazine or deuterated tetrabenazine can be administered to a subject in need thereof to achieve a therapeutic effect at a desired dose and/or with a suitable PK profile described herein. Any of the transdermal delivery device disclosed herein can be useful. In some embodiments, the present disclosure provides the following specific embodiments, SE1 to SE8.

SE1: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to transdermally deliver to the subject about 0.1 mg/day to about 20 mg/day of R,R-tetrabenazine, for example, about 0.5 mg/day to about 10 mg/day of R,R-tetrabenazine, about 0.5 mg/day to about 8 mg/day of R,R-tetrabenazine, about 0.5 mg/day to about 6 mg/day of R,R-tetrabenazine, about 0.5 mg/day to about 4 mg/day of R,R-tetrabenazine, about 1 mg/day to about 10 mg/day of R,R-tetrabenazine, about 1 mg/day to about 8 mg/day of R,R-tetrabenazine, about 1 mg/day to about 6 mg/day of R,R-tetrabenazine, about 1 mg/day to about 4 mg/day of R,R-tetrabenazine, about 2 mg/day to about 10 mg/day of R,R-tetrabenazine, about 2 mg/day to about 8 mg/day of R,R-tetrabenazine, about 2 mg/day to about 6 mg/day of R,R-tetrabenazine, about 2 mg/day to about 4 mg/day of R,R-tetrabenazine, about 4 mg/day to about 10 mg/day of R,R-tetrabenazine, about 4 mg/day to about 8 mg/day of R,R-tetrabenazine, about 4 mg/day to about 6 mg/day of R,R-tetrabenazine, about 6 mg/day to about 10 mg/day of R,R-tetrabenazine, or about 6 mg/day to about 8 mg/day of R,R-tetrabenazine. In some embodiments according to SE1, the transdermal delivery device is applied to transdermally deliver to the subject about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine. In some embodiments according to SE1, the drug-in-adhesive layer can include an optional permeation enhancer and/or a plasticizer.

SE2: A method of treating a hyperkinetic movement disorder in a subject in need thereof, the method comprising applying to the subject a transdermal delivery device described herein, preferably, a transdermal delivery device comprising a drug-in-adhesive layer, wherein the drug-in-adhesive layer comprises (1) an active ingredient comprising R,R-tetrabenazine, (2) a pressure sensitive adhesive herein, e.g., a non-reactive acrylate pressure sensitive adhesive; (3) an optional crystallization inhibitor; and (4) an optional antioxidant; wherein the transdermal delivery device provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.) or the ratio of the steady state plasma concentration of R,R-tetrabenazine to the combined steady state plasma concentration of R,R,R-HTBZ and S,R,R-HTBZ ranges from about 1:1 to about 1:5 (e.g., about 1:1, about 1:1.2, about 1:1.5, about 1:2, about 1:3, about 1:4, about 1:5, or any ranges between the recited values, for example, about 1:1 to about 1:3, about 1:2 to about 1:4, etc.). In some embodiments according to SE2, the ratio of maximum plasma concentration or steady state plasma concentration of R,R,R-HTBZ to S,R,R-HTBZ ranges from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments according to SE2, the ratio of maximum plasma concentration or steady state plasma concentration of R,R-tetrabenazine:R,R,R-HTBZ:S,R,R-HTBZ is about 17-40:3-10:50-80. In some embodiments according to SE2, the applying can provide a pharmacokinetic profile characterized in that the ratio of AUC_{0-∞} of SRR-HTBZ to the AUC_{0-∞} of R,R-tetrabenazine is about 1 to about 15, e.g., about 1.5 to about 11, and/or the ratio of AUC_{0-∞} of RRR-HTBZ to the AUC_{0-∞} of R,R-tetrabenazine is about 0.1 to about 0.75, e.g., about 0.15 to about 0.5. In some embodiments according to SE2, the administering provides no detectable S,S-tetrabenazine, R,S,S-HTBZ, or S,S,S-HTBZ in the plasma of the subject. In some embodiments according to SE2, the PK profile is characterized in that the average terminal half-life of R,R-tetrabenazine is about 8.5 hr±40% CV. In some embodiments according to SE2, the drug-in-adhesive layer can include an optional permeation enhancer and/or a plasticizer. In some embodiments according to SE2, the transdermal delivery device is applied to transdermally deliver to the subject any of the daily dose described in SE1. In some embodiments according to SE2, the transdermal delivery device is applied to achieve any of the PK profiles described herein in any combination.

SE3: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to the subject to achieve a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments according to SE3, the drug-in-adhesive layer can include an optional permeation enhancer and/or a plasticizer. In some embodiments according to SE3, the transdermal delivery device is applied to transdermally deliver to the subject any of the daily dose described in SE1. In some embodiments according to SE3, the transdermal delivery device is applied to achieve any of the PK profiles in the subject described in SE2.

SE4: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to the subject to achieve a substantially constant steady state plasma concentration of R,R-tetrabenazine above 150 pg/ml, for a sustained period of at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments according to SE4, the transdermal delivery device is applied to the subject to achieve a substantially constant steady state plasma concentration of R,R-tetrabenazine above 150 pg/ml to about 3000 pg/ml for the sustained period, wherein the plasma concentration of R,R-tetrabenazine does not vary significantly, e.g., by more than two-fold, during any of the 4-hour, 8-hour, and/or 12-hour intervals of the sustained period. In some embodiments according to SE4, during the sustained period, the ratio of the lowest plasma concentration of R,R-tetrabenazine to the maximum plasma concentration of R,R-tetrabenazine is no less than about 0.4, such as about 0.5, about 0.6, about 0.7, about 0.8, about 0.9, about 1, or any ranges between the recited values. In some embodiments according to SE4, the drug-in-adhesive layer can include an optional permeation enhancer and/or a plasticizer. In some embodiments according to SE4, the transdermal delivery device is applied to transdermally deliver to the subject any of the daily dose described in SE1. In some embodiments according to SE4, the transdermal delivery device is applied to achieve any of the PK profiles in the subject described in SE2.

SE5: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising a deuterated R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to transdermally deliver to the subject about 0.1 mg/day to about 20 mg/day of deuterated R,R-tetrabenazine, for example, about 0.5 mg/day to about 10 mg/day of deuterated R,R-tetrabenazine, about 0.5 mg/day to about 8 mg/day of deuterated R,R-tetrabenazine, about 0.5 mg/day to about 6 mg/day of deuterated R,R-tetrabenazine, about 0.5 mg/day to about 4 mg/day of deuterated R,R-tetrabenazine, about 1 mg/day to about 10 mg/day of deuterated R,R-tetrabenazine, about 1 mg/day to about 8 mg/day of deuterated R,R-tetrabenazine, about 1 mg/day to about 6 mg/day of deuterated R,R-tetrabenazine, about 1 mg/day to about 4 mg/day of deuterated R,R-tetrabenazine, about 2 mg/day to about 10 mg/day of deuterated R,R-tetrabenazine, about 2 mg/day to about 8 mg/day of deuterated R,R-tetrabenazine, about 2 mg/day to about 6 mg/day of deuterated R,R-tetrabenazine, about 2 mg/day to about 4 mg/day of deuterated R,R-tetrabenazine, about 4 mg/day to about 10 mg/day of deuterated R,R-tetrabenazine, about 4 mg/day to about 8 mg/day of deuterated R,R-tetrabenazine, about 4 mg/day to about 6 mg/day of deuterated R,R-tetrabenazine, about 6 mg/day to about 10 mg/day of deuterated R,R-tetrabenazine, or about 6 mg/day to about 8 mg/day of deuterated R,R-tetrabenazine. In some embodiments according to SE5, the transdermal delivery device is applied to transdermally deliver to the subject about 0.5 mg/day to about 10 mg/day of deuterated R,R-tetrabenazine, about 0.5 mg/day to about 8 mg/day of deuterated R,R-tetrabenazine, or about 2 mg/day to about 6 mg/day of deuterated R,R-tetrabenazine. In some embodiments according to SE5, the drug-in-adhesive layer can include an optional permeation enhancer and/or a plasticizer.

SE6. A method of treating a hyperkinetic movement disorder in a subject in need thereof, the method comprising applying to the subject a transdermal delivery device described herein, preferably, a transdermal delivery device comprising a drug-in-adhesive layer, wherein the drug-in-adhesive layer comprises (1) an active ingredient comprising a deuterated R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device provides a therapeutically effective plasma concentration of deuterated R,R-tetrabenazine, the dihydrotetrabenazine metabolite of the deuterated R,R-tetrabenazine, deuterated R,R,R-HTBZ and deuterated S,R,R-HTBZ, wherein the ratio of the maximum plasma concentration of the deuterated R,R-tetrabenazine to the combined maximum plasma concentration of the deuterated R,R,R-HTBZ and deuterated S,R,R-HTBZ ranges from about 1:1 to about 1:7.5 (e.g., about 1:1, about 1:1.2, about 1:1.5, about 1:2, about 1:3, about 1:4, about 1:5, about 1:7.5 or any ranges between the recited values, for example, about 1:1 to about 1:3, about 1:2 to about 1:4, etc.) or the ratio of the steady state plasma concentration of the deuterated R,R-tetrabenazine to the combined maximum plasma concentration of the deuterated R,R,R-HTBZ and deuterated S,R,R-HTBZ ranges from about 1:1 to about 1:7.5. In some embodiments according to SE6, the ratio of maximum plasma concentration of deuterated R,R,R-HTBZ to deuterated S,R,R-HTBZ can range from about 1:5 to about 1:30 (e.g., about 1:5, about 1:7, about 1:9, about 1:10, about 1:15, about 1:20, about 1:30, or any ranges between the recited values, for example, from about 1:10 to about 1:20, about 1:5 to about 1:15, etc.). In some embodiments according to SE6, the ratio of maximum plasma concentration or steady state plasma concentration of deuterated R,R-tetrabenazine:deuterated R,R,R-HTBZ:deuterated S,R,R-HTBZ can range from about 14-40:3-11:50-85. In some embodiments according to SE6, the administering can provide a pharmacokinetic profile characterized in that the ratio of AUC_{0-∞} of deuterated SRR-HTBZ to the AUC_{0-∞} of deuterated R,R-tetrabenazine is about 1 to about 15, such as about 1.5 to about 11, and/or the ratio of AUC_{0-∞} of deuterated RRR-HTBZ to the AUC_{0-∞} of deuterated R,R-tetrabenazine is about 0.1 to about 0.75, such as about 0.15 to about 0.5. In some embodiments according to SE6, the administering provides no detectable deuterated S,S-tetrabenazine, deuterated R,S,S-HTBZ, or deuterated S,S,S-HTBZ in the plasma of the subject. In some embodiments according to SE6, the PK profile is characterized in that the average terminal half-life of deuterated R,R-tetrabenazine is about 8.5 hr+40% CV. In some embodiments according to SE6, the drug-in-adhesive layer can include an optional permeation enhancer and/or a plasticizer. In some embodiments according to SE6, the transdermal delivery device is applied to transdermally deliver to the subject any of the daily dose described in SE5. In some embodiments according to SE6, the transdermal delivery device is applied to achieve any of the PK profiles described herein.

SE7: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising deuterated R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to the subject to achieve a therapeutically effective plasma concentration of deuterated R,R-tetrabenazine, the dihydrotetrabenazine metabolite of the deuterated R,R-tetrabenazine, deuterated R,R,R-HTBZ and deuterated S,R,R-HTBZ, for at least 6 hours, or at least 12 hours, preferably, at least 24 hours, for example, at least 24 hours, at least 48 hours, at least 72 hours, at least 96 hours, at least 120 hours, at least 144 hours, at least 168 hours, at least 192 hours, or more. In some embodiments according to SE7, the drug-in-adhesive layer can include an optional permeation enhancer and/or a plasticizer. In some embodiments according to SE7, the transdermal delivery device is applied to transdermally deliver to the subject any of the daily dose described in SE5. In some embodiments according to SE7, the transdermal delivery device is applied to achieve any of the PK profiles described in SE6.

SE8: A method of treating a hyperkinetic movement disorder in a subject in need thereof, comprising applying to the subject a transdermal delivery device described herein, preferably, a transdermal delivery device comprising a drug-in-adhesive layer, wherein the drug-in-adhesive layer comprises (1) an active ingredient comprising a deuterated R,R-tetrabenazine, (2) a pressure sensitive adhesive herein, e.g., a non-reactive acrylate pressure sensitive adhesive; (3) an optional crystallization inhibitor; and (4) an optional antioxidant, wherein the transdermal delivery device is applied to the subject to achieve a substantially constant steady state plasma concentration of the deuterated R,R-tetrabenazine above 150 pg/ml, for a sustained period of at least 6 hours, or at least 12 hours, preferably, at least 24 hours for example, at least 24 hours, at least 48 hours, at least 72 hours, at least 96 hours, at least 120 hours, at least 144 hours, at least 168 hours, at least 192 hours, or more. In some embodiments according to SE8, the transdermal delivery device is applied to the subject to achieve a substantially constant steady state plasma concentration of deuterated R,R-tetrabenazine above 150 pg/ml to about 3000 pg/ml for the sustained period, wherein the plasma concentration of deuterated R,R-tetrabenazine does not vary significantly, e.g., by more than two-fold, during any of the 4-hour, 8-hour, and/or 12-hour intervals of the sustained period. In some embodiments according to SE8, during the sustained period, the ratio of the lowest plasma concentration of deuterated R,R-tetrabenazine to the maximum plasma concentration of deuterated R,R-tetrabenazine is no less than about 0.4, such as about 0.5, about 0.6, about 0.7, about 0.8, about 0.9, about 1, or any ranges between the recited values. In some embodiments according to SE8, the drug-in-adhesive layer can include an optional permeation enhancer and/or a plasticizer. In some embodiments according to SE8, the transdermal delivery device is applied to transdermally deliver to the subject any of the daily dose described in SE5. In some embodiments according to SE8, the transdermal delivery device is applied to achieve any of the PK profiles in the subject described in SE6.

Any of the transdermal delivery device designs described herein can be used for the methods described. Typically, for any of the embodiments according to SE1 to SE8, the transdermal delivery device can be a DIA patch, which comprises the drug-in-adhesive layer, a backing layer, and a release liner which protects the adhesive surface prior to use. The drug-in-adhesive layer typically includes the active ingredient dispersed in the adhesive, preferably, homogeneously dispersed. In some embodiments, the transdermal delivery device is applied to the subject to deliver about 0.1 mg/day to about 20 mg/day of R,R-tetrabenazine (e.g., any of the exemplified ranges described herein) at a substantially constant rate for up to 24 hours post application, up to 48 hours post application, up to 96 hours post application, or up to 1 week post application. Suitable composition for the drug-in-adhesive layer include any of those adhesive compositions described herein, e.g., any of those described in Embodiments 1-18 as applicable to tetrabenazine or deuterated tetrabenazine or any of the specific compositions shown in the Examples section (e.g., Example 4A). In some embodiments, the drug-in-adhesive layer comprises the active ingredient dispersed in a non-reactive acrylate pressure sensitive adhesive. The active ingredient can be present in an amount of about 1% to about 20% by weight, such as about 1%, about 2%, about 3%, about 4%, about 5%, about 6%, about 7%, about 10%, about 15%, about 20%, or any ranges between the recited values, for example, about 1% to about 15%, about 2% to about 15%, about 2% to about 10%, about 2% to about 7%, about 3% to about 15%, about 3% to about 10%, about 3% to about 7%, about 5% to about 15%, about 5% to about 10%, about 5% to about 7%, about 7% to about 15%, about 7% to about 10%, about 10% to about 20%, about 10% to about 15%, etc. In some embodiments, the drug-in-adhesive layer comprises a substantially pure R,R-isomer of tetrabenazine as the sole active ingredient. In some embodiments, the drug-in-adhesive layer comprises a substantially pure deuterated R,R-isomer of tetrabenazine as the sole active ingredient. Typically, the R,R-isomer of tetrabenazine or the deuterated R,R-isomer of tetrabenazine are present in its free base form. Suitable adhesive include any of those described herein, such as any of the pressure sensitive adhesive described herein. In some embodiments, the adhesive can be a non-reactive acrylate pressure sensitive adhesive described herein, such as Duro-Tak 87-900A or described in exemplary Embodiments 2-7. The adhesive typically is present in an amount of about 50% to about 97% by weight, such as about 50%, about 60%, about 70%, about 80%, about 90%, about 95%, about 97%, or any ranges between the recited values, for example, about 50% to about 95%, about 50% to about 90%, about 50% to about 80%, about 50% to about 70%, about 50% to about 60%, about 60% to about 97%, about 60% to about 95%, about 60% to about 90%, about 60% to about 80%, about 60% to about 70%, about 70% to about 97%, about 70% to about 95%, about 70% to about 90%, about 70% to about 80%, about 80% to about 97%, about 80% to about 95%, about 80% to about 90%, etc. In some specific embodiments, the active ingredient is in an amount of about 2% to about 7% by weight and the non-reactive acrylate pressure sensitive adhesive is in an amount of about 50% to about 97% by weight. The adhesive composition can also optionally include other ingredients, such as an antioxidant, a crystallization inhibitor, a plasticizer, and/or a permeation enhancer. In some embodiments, the drug-in-adhesive layer includes an antioxidant, such as a gallate antioxidant, for example, propyl gallate. The amount of antioxidant is typically included in an amount of about 0% to about 1% by weight, such as about 0.001%, about 0.01%, about 0.1%, about 0.5%, about 1%, or any ranges between the recited values, for example, about 0.001% to about 0.5%, about 0.01% to about 0.5%, etc. by weight. In some embodiments, the drug-in-adhesive layer includes a crystallization inhibitor, for example, a means to prevent formation of drug crystals (crystals of the active ingredient of the drug-in-adhesive layer, such as R,R-tetrabenazine) after shelf storage for two weeks at ambient temperature. In some embodiments, the adhesive composition includes a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. In some embodiments, the drug-in-adhesive layer includes a crystallization inhibitor selected from a polymethacrylate (e.g., Plastoid B (copolymer of butyl methacrylate and methyl methacrylate), Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof. The crystallization inhibitor is typically present in an amount of about 0 to about 40% by weight, such as about 5%, about 10%, about 15%, about 20%, about 30%, about 40%, or any range between the recited values, for example, about 10% to about 40%, about 10% to about 30%, about 10% to about 20%, 15% to about 40%, about 15% to about 30%, about 15% to about 20%, 20% to about 40%, about 20% to about 30%, etc. by weight. In some embodiments, the drug-in-adhesive layer can also comprise a skin permeation enhancer as described herein, such as isopropyl myristate. In some embodiments, the drug-in-adhesive layer can also be free of a skin permeation enhancer as described herein, for example, in some embodiments, the drug-in-adhesive layer can also be free of isopropyl myristate. The weight and thickness of the drug-in-adhesive layer can vary depending on different factors such as drug concentration and desired dose and duration of administration, etc. The drug-in-adhesive layer is typically designed for application (e.g., delivering R,R-tetrabenazine or deuterated R,R-trabenazine) for a period of time selected from about 8 hours, about 12 hours, about 18 hours, about 24 hours, about 2 days, about 3 days, about 4 days, about 5 days, about 6 days, and about 7 days. In some embodiments, the drug-in-adhesive layer can have a coat weight of about 0.1 g/cm² to about 5 g/cm², such as about 0.1 g/cm² to about 0.90 g/cm² (e.g., about 0.1 g/cm² to about 0.5 g/cm²) active surface area. In some embodiments, the active surface area can be from 5 cm² to about 100 cm² (other suitable ranges are described herein). In some embodiments, the drug-in-adhesive layer can have a thickness of about 1.5 mils to about 10 mils, such as about 1.5 mils to about 3.5 mils (e.g., about 2 mils to about 3.5 mils). In some embodiments, the transdermal delivery device can also be configured to have any of the in vitro flux characteristics described herein.

The hyperkinetic movement disorder recited in the methods according to any one of SE1 to SE8 include any of those described herein. Non-limiting examples include Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof. In some embodiments, the hyperkinetic movement disorder can be Huntington's disease, such as chorea associated with Huntington's disease. In some embodiments, the hyperkinetic movement disorder can be Wilson's disease. In some embodiments, the hyperkinetic movement disorder can be Tourette syndrome. In some embodiments, the hyperkinetic movement disorder can be restless leg syndrome. In some embodiments, the hyperkinetic movement disorder can be tardive dyskinesia. In some embodiments, the hyperkinetic movement disorder can be tic. In some embodiments, the hyperkinetic movement disorder can be dyskinetic cerebral palsy. In some embodiments, the hyperkinetic movement disorder is a dystonia or dyskinesia disorder.

The methods of treatment according to any one of SE1 to SE8 are not limited to any specific type of subjects. For example, the method herein can be administered to the subject without regard to the fed status of the subject. In some embodiments, the subject is a pediatric and adolescent patient (e.g., 6 through 18 years of age). Also, in some embodiments, the method is not limited to any specific genotyped subjects. In some embodiments, the same dose or substantially the same dose of R,R-tetrabenazine or a deuterated R,R-tetrabenazine can be administered to subjects who are characterized as PM, IM, or EM. In some embodiments, the subject is characterized as EM. In some embodiments, the subject is characterized as PM. In some embodiments, the subject is characterized as IM. In some embodiments, the method does not require dose titration and/or genotype analysis, which is required when treated with either of Xenazine® and Austedo™ tablets.

The dosing regimen for the methods of treatment according to any one of SE1 to SE8 is not particularly limited, so long as the desired dose of R,R-tetrabenazine or deuterated R,R-tetrabenazine is delivered to the subject for a desired period of time at a desired rate, which includes any of those described herein. For example, in some embodiments, the transdermal delivery device is applied to the subject in a dosing regimen suitable to achieve the PK profiles herein. In some embodiments, the transdermal delivery device is applied to the subject once a day. In some embodiments, the transdermal delivery device is applied to the subject once in more than a day, such as once in two days, once in three days, once in four days, once in five days, once in six days, once in a week, or once in more than a week. In some embodiments, for each application, the transdermal delivery device can be applied to the skin of the subject to adhere to the skin of the subject for about 8 hours, about 12 hours, about 24 hours, about 36 hours, about 48 hours, about 72 hours, about 96 hours, about 120 hours, about 144 hours, about 168 hours, about 192 hours, or any range between the recited values, which can deliver R,R-tetrabenazine continuously or substantially continuously to the subject during the patch-on period. In some embodiments, there is no lag time between two consecutive applications of transdermal patches according to the dosing frequency herein. For example, in some embodiments, the transdermal delivery device is applied to the subject once a day, and each application of the transdermal delivery device can last (adhere to the skin of the subject) for about 24 hours before it is replaced with another transdermal delivery device which is typically substantially same. However, in some embodiments, there can also be overlap or lag between two consecutive applications.

### Combination Therapy

The methods herein can use tetrabenazine and/or deuterated tetrabenazine as the only active ingredient(s). In other words, the methods herein can be used as a monotherapy. However, in some embodiments, the methods herein can also be used in combination with one or more additional therapy. The transdermal delivery methods, devices, and pharmaceutical compositions for combination therapy are illustrated below as examples.

In some embodiments, the transdermal delivery devices or pharmaceutical compositions disclosed herein (e.g., adhesive compositions herein) may also be combined or used in combination with other agents useful in the treatment of VMAT2-mediated disorders. Such other agents can be administered, by a route and in an amount commonly used therefor, simultaneously or sequentially with the transdermal delivery devices or pharmaceutical compositions disclosed herein (e.g., adhesive compositions herein). In some embodiments, such other agents are included in the transdermal delivery devices or pharmaceutical compositions disclosed herein (e.g., adhesive compositions herein). However, in some embodiments, such other agents are administered as a separate composition or otherwise independent of the transdermal delivery devices or pharmaceutical compositions disclosed herein (e.g., adhesive compositions herein).

In some embodiments, the transdermal delivery devices or pharmaceutical compositions disclosed herein (e.g., adhesive compositions herein) can be used in combination with one or more anti-psychotics, including, but not limited to, chlorpromazine, levomepromazine, promazine, acepromazine, triflupromazine, cyamemazine, chlorproethazine, dixyrazine, fluphenazine, perphenazine, prochlorperazine, thiopropazate, trifluoperazine, acetophenazine, thioproperazine, butaperazine, perazine, periciazine, thioridazine, mesoridazine, pipotiazine, haloperidol, trifluperidol, melperone, moperone, pipamperone, bromperidol, benperidol, droperidol, fluanisone, oxypertine, molindone, sertindole, ziprasidone, flupentixol, clopenthixol, chlorprothixene, thiothixene, zuclopenthixol, fluspirilene, pimozide, penfluridol, loxapine, clozapine, olanzapine, quetiapine, tetrabenazine, sulpiride, sultopride, tiapride, remoxipride, amisulpride, veralipride, levosulpiride, lithium, prothipendyl, risperidone, clotiapine, mosapramine, zotepine, pripiprazole, and paliperidone.

In some embodiments, the transdermal delivery devices or pharmaceutical compositions disclosed herein (e.g., adhesive compositions herein) can be used in combination with one or more benzodiazepines (“minor tranquilizers”), including, but not limited to alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

In some embodiments, the transdermal delivery devices or pharmaceutical compositions disclosed herein (e.g., adhesive compositions herein) can be used in combination with olanzapine or pimozide.

In some embodiments, the transdermal delivery devices or pharmaceutical compositions disclosed herein (e.g., adhesive compositions herein) can be used in combination with other classes of compounds, including, but not limited to, anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs), such as naproxen; antibacterial agents, such as amoxicillin; cholesteryl ester transfer protein (CETP) inhibitors, such as anacetrapib; anti-fungal agents, such as isoconazole; sepsis treatments, such as drotrecogin-α; steroidals, such as hydrocortisone; local or general anesthetics, such as ketamine; norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; famesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporine; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

In some embodiments, the transdermal delivery devices or pharmaceutical compositions disclosed herein (e.g., adhesive compositions herein) can be used in combination with dextromethorphan, and/or a cannabinoid, such as cannabidiol.

### Definitions

As used herein, the term “about” modifying an amount related to the invention refers to variation in the numerical quantity that can occur, for example, through routine testing and handling; through inadvertent error in such testing and handling; through differences in the manufacture, source, or purity of ingredients/materials employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

As used herein, “coat weight” of a drug layer refers to the weight of the drug layer (e.g., a drug-in-adhesive layer or a drug-in-reservoir layer) per unit area of the active surface area of the transdermal drug delivery system.

As used herein, the term “cumulative drug permeated” refers to the total amount of drug permeated per square centimeter during a given period of time. Unless otherwise obvious from context, “cumulative drug permeated” at a given time (e.g., at 24 hours post administration) refers to the total amount of drug permeated per square centimeter from time 0 (i.e., time of administration) to the given time. Unless otherwise obvious from context, “cumulative drug permeated” refers to the arithmetic mean value measured and/or calculated in accordance with the methods described herein. The term “mean value” as used herein, when not specified, also refers to arithmetic mean value, unless contradictory to common practice in the field.

As used herein, the term “flux” refers to the quantity of the drug permeated skin per unit area per unit time. Unless otherwise obvious from context, “flux” refers to the arithmetic mean value measured and/or calculated in accordance with the methods described herein. A typical unit of flux is milligram per square centimeter per hour.

Flux rate as referenced in this patent application can mean that measured by either in vivo or in vitro methods. One way to measure flux is to place the transdermal delivery device or formulation on a known skin area of a human volunteer and measure how much drug can permeate across skin within certain time constraints. In some embodiments, when specifically referenced as measured by in vitro method using human cadaver skin, the flux rate is measured in accordance with the method described in Example 3 or 6. Although an in vitro method uses human epidermal membrane obtained from a cadaver, rather than measure drug flux across the skin using human volunteers, it is generally accepted by those skilled in the art that results from a properly designed and executed in vitro test can be used to estimate or predict the results of an in vivo test with reasonable reliability.

The terms “skin flux characteristics” and “flux characteristics” are used interchangeably herein.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., tetrabenazine) sufficient to result in amelioration of one or more symptoms of a disorder or condition (e.g., Huntington's disease), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

As used herein, applying or administering the transdermal delivery device herein should be understood as in accordance with how such transdermal delivery device is normally applied or administered, e.g., to the skin of a human subject.

The term “chronic hyperkinetic movement disorders” refers to disorders characterized by non-purposeful, repetitive, disordered motor acts, variously termed “compulsive”, “rhythmical”, or “stereotyped.” In humans, chronic hyperkinetic movement disorders can be psychogenic (e.g., tics), idiopathic (as in, e.g., Tourette's syndrome and Parkinson's Disease, genetic (as in, e.g., the chorea characteristic of Huntington's Disease), infectious (as in, e.g., Sydenham's Chorea), or, as in tardive dyskinesia, drug-induced. Unless otherwise stated, “chronic hyperkinetic movement disorders” refers to and includes all psychogenic, idiopathic, genetic, and drug-induced movement disorders.

The term “stereotyped” refers to a repeated behavior that appears repetitively with slight variation or, less commonly, as a complex series of movements.

The term “VMAT2” refers to vesicular monoamine transporter 2, an integral membrane protein that acts to transport monoamines—particularly neurotransmitters such as dopamine, norepinephrine, serotonin, and histamine—from cellular cytosol into synaptic vesicles.

The term “VMAT2-mediated disorder,” refers to a disorder that is characterized by abnormal VMAT2 activity. A VMAT2-mediated disorder may be completely or partially mediated by modulating VMAT2. In particular, a VMAT2-mediated disorder is one in which inhibition of VMAT2 results in some effect on the underlying disorder e.g., administration of a VMAT2 inhibitor results in some improvement in at least some of the patients being treated.

### Exemplary Embodiments 1-30

The following shows certain exemplary embodiments (Embodiments 1-30) of the present disclosure.

Embodiment 1. An adhesive composition comprising:
an active ingredient dispersed in a non-reactive acrylate pressure sensitive adhesive, wherein the active ingredient is selected from tetrabenazine, deuterated tetrabenazine, or a combination thereof,
wherein the non-reactive acrylate pressure sensitive adhesive is in an amount of about 50% to about 97% by weight.
   Embodiment 2. The adhesive composition of embodiment 1, wherein the non-reactive acrylate pressure sensitive adhesive does not have functional groups containing reactive hydrogen moieties.
   Embodiment 3. The adhesive composition of embodiment 1, wherein the non-reactive acrylate pressure sensitive adhesive does not have functional groups selected from epoxy, —OH, —COOH, and combinations thereof.
   Embodiment 4. The adhesive composition of any one of embodiments 1-3, wherein the non-reactive acrylate pressure sensitive adhesive is a copolymer of alkyl acrylate.
   Embodiment 5. The adhesive composition of any one of embodiments 1-3, wherein the non-reactive acrylate pressure sensitive adhesive is a copolymer of C₂-C₁₈ alkyl acrylate (preferably C₄-C₁₀ branched or straight-chain alkyl acrylate) and methyl acrylate, and optionally one or more acrylamide monomers (e.g., tert octyl acrylamide) with no functional groups selected from epoxy, —OH, —COOH, and combinations thereof.
   Embodiment 6. The adhesive composition of any one of embodiments 1-3, wherein the non-reactive acrylate pressure sensitive adhesive is a copolymer of hexylethyl acrylate (e.g., 2-ethyl hexyl acrylate) and methyl acrylate, and optionally one or more acrylamide monomers (e.g., tert octyl acrylamide) with no functional groups selected from epoxy, —OH, —COOH, and combinations thereof.
   Embodiment 7. The adhesive composition of any one of embodiments 1-6, wherein the non-reactive acrylate pressure sensitive adhesive is free or substantially free of vinyl acetate.
   Embodiment 8. The adhesive composition of any one of embodiments 1-7, which is free of a permeation enhancer.
   Embodiment 9. The adhesive composition of any one of embodiments 1-7, which is free of isopropyl myristate.
   Embodiment 10. The adhesive composition of any one of embodiments 1-7, which is free of a permeation enhancer selected from fatty alcohols, fatty acids, fatty esters and combinations thereof.
   Embodiment 11. The adhesive composition of any one of embodiments 1-10, wherein the active ingredient is present in an amount of about 2% to about 7% by weight.
   Embodiment 12. The adhesive composition of any one of embodiments 1-11, further comprising a gallate antioxidant.
   Embodiment 13. The adhesive composition of any one of embodiments 1-11, further comprising propyl gallate, e.g., in an amount of about 0.001% to about 0.5% by weight.
   Embodiment 14. The adhesive composition of any one of embodiments 1-13, further comprising a crystallization inhibitor in an amount effective to prevent formation of drug crystals after shelf storage for two weeks at ambient temperature.
   Embodiment 15. The adhesive composition of any one of embodiments 1-13, further comprising a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof.
   Embodiment 16. The adhesive composition of any one of embodiments 1-13, further comprising a crystallization inhibitor selected from a polymethacrylate (e.g., Plastoid B (copolymer of butyl methacrylate and methyl methacrylate), Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof.
   Embodiment 17. The adhesive composition of any one of embodiments 1-16, wherein the sole active ingredient is a substantially pure R,R-isomer of tetrabenazine.
   Embodiment 18. The adhesive composition of any one of embodiments 1-17, which is capable of adhering continuously to the skin of a user for about 8 hours, about 12 hours, about 18 hours, about 24 hours, about 2 days, about 3 days, about 4 days, about 5 days, about 6 days, or about 7 days or more.
   Embodiment 19. A transdermal delivery device comprising:
a backing layer,
the adhesive composition of any one of embodiments 1-18; and
a release liner.
   Embodiment 20. The transdermal delivery device of embodiment 19, which is shelf stable.
   Embodiment 21. The transdermal delivery device of embodiment 19 or 20, which provides a subject user the active ingredient at a rate of about 0.01 mg/day/cm² to about 5 mg/day/cm², e.g., for a period of about 8 hours, about 12 hours, about 18 hours, about 24 hours, about 2 days, about 3 days, about 4 days, about 5 days, about 6 days, or about 7 days or more.
   Embodiment 22. A method of administering tetrabenazine, deuterated tetrabenazine, or a combination thereof, to a subject in need thereof, comprising applying the adhesive composition of any one of embodiments 1-18 or the transdermal delivery device of any one of embodiments 19-21 to the skin of the subject.
   Embodiment 23. The method of embodiment 22, wherein the subject is characterized as having a hyperkinetic movement disorder (e.g., a chronic hyperkinetic movement disorder).
   Embodiment 24. The method of embodiment 23, wherein the hyperkinetic movement disorder is chorea associated with Huntington's disease, Wilson's disease, Tourette syndrome, restless leg syndrome, tardive dyskinesia, tic, and combinations thereof.
   Embodiment 25. A method of inhibiting a vesicular monoamine transporter isoform 2 (VMAT2) in a subject in need thereof, the method comprising applying the adhesive composition of any one of embodiments 1-18 or the transdermal delivery device of any one of embodiments 19-21 to the skin of the subject.
   Embodiment 26. A method of treating a vesicular monoamine transporter isoform 2 (VMAT2) mediated disease or disorder in a subject in need thereof, the method comprising applying the adhesive composition of any one of embodiments 1-18 or the transdermal delivery device of any one of embodiments 19-21 to the skin of the subject.
   Embodiment 27. A method of treating a hyperkinetic movement disorder in a subject in need thereof, comprising applying the adhesive composition of any one of embodiments 1-18 or the transdermal delivery device of any one of embodiments 19-21 to the skin of the subject.
   Embodiment 28. The method of embodiment 27, wherein the hyperkinetic movement disorder is a chronic hyperkinetic movement disorder.
   Embodiment 29. The method of embodiment 27 or 28, wherein the hyperkinetic movement disorder is chorea associated with Huntington's disease, Wilson's disease, Tourette syndrome, restless leg syndrome, tardive dyskinesia, and/or a tic.
   Embodiment 30. A method of treating a hyperkinetic movement disorder in a subject in need thereof, the method comprising transdermally administering a therapeutically effective amount of tetrabenazine or deuterated tetrabenazine to the subject.

### EXAMPLES

### Example 1. Preparation of Tetrabenazine Transdermal Patch

This example shows one procedure for preparing tetrabenazine drug-in-adhesive patch. Tetrabenazine base is generally commercially available in high purity (e.g., 99%), for example, from Octagon Chemical Ltd, Hangzhou, China, via internet on “Alibaba” website.

In this example, tetrabenazine base was thoroughly mixed into the adhesive, Durotak 87-2287 (manufactured by Henkel Adhesives) until the mixture was homogenous. Afterwards, the adhesive mixture was dispensed on a release liner using the “draw down knife” and forced dried for 1.5 min using a typical hair drier followed by lamination to a backing film.

The adhesive mixtures can contain tetrabenazine in different concentrations. In this example, four concentrations were used: 1) 2.5% formulation, prepared from mixing 2.5% tetrabenazine with 97.5% Durotak 87-2287; 2) 5% formulation, prepared from mixing 5% tetrabenazine with 95% Durotak 87-2287; 3) 10% formulation, prepared from mixing 10% tetrabenazine with 90% Durotak 87-2287; and 4) 15% formulation, prepared from mixing 15% tetrabenazine with 85% Durotak 87-2287. All percentages for tetrabenazine and Durotak refer to weight percentage based on final weight of the respective formulations.

### Example 2. Tetrabenazine Transdermal Patch with Two Adhesives

Following the same procedure of Example 1, tetrabenazine transdermal patches with a mixture of two different adhesives at different ratios were also prepared, with the concentration of tetrabenazine being kept at 10% by weight.

The two adhesives used in this Example were a silicone polymer (BIO-7-4202 from Dow Corning Co.) and an acrylate copolymer (Durotak 87-2287). Four different ratios were used in this example: 1) 5/95 (Durotak 87-2287/BIO-7-4202); 2) 10/90 (Durotak 87-2287/BIO-7-4202); 3) 25/75 (Durotak 87-2287/BIO-7-4202); and 4) 50/50 (Durotak 87-2287/BIO-7-4202).

### Example 3. Transdermal Flux Test

Transdermal flux of Tetrabenazine from the patch was tested using human cadaver epidermis by Franz Diffusion Cell method. The cadaver epidermis was obtained from Health Science Tissue Bank in Phoenix Ariz.

The transdermal flux of Tetrabenazine through human cadaver epidermis was analyzed using the following HPLC method.

Mobile Phase: 55/45 Acetonitrile/Water—0.05% Triethylamine adjusted pH-6.5
HPLC Column: Kinetex C18, 150×4.6 mm, 5 m from Pheneomenex
Wavelength: 230 nm, Flow: 1.2 mL/min

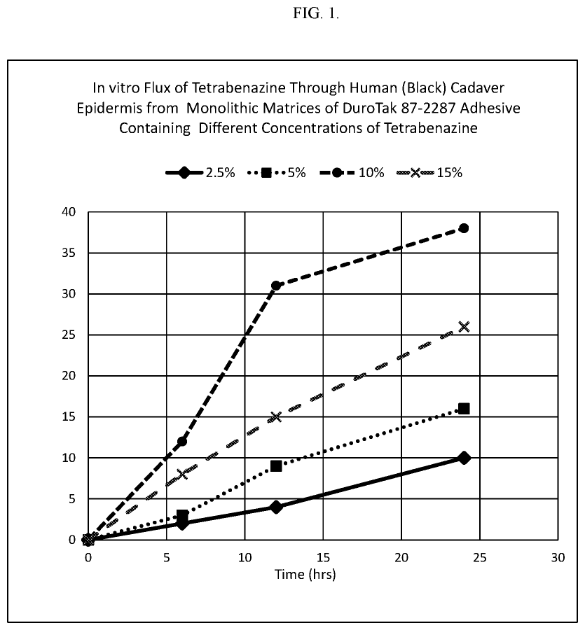

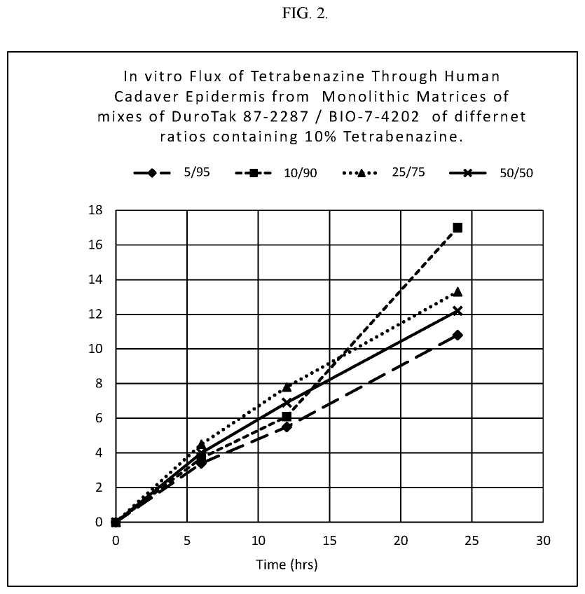

The patches in Examples 1 and 2 were tested and the transdermal flux results are presented in FIGS. 1 and 2, respectively.

As shown in FIG. 1, the highest transdermal flux observed for patches in Example 1 is the adhesive matrix with 10% Tetrabenazine concentration.

As shown in FIG. 2, varying the ratio of the silicone polymer to acrylate copolymer does not significantly change the flux characteristics. However, when compared to the results shown in FIG. 1, the flux observed for patches prepared according to Example 2 is noticeably lower than the flux from the patch with 10% Tetrabenazine concentration prepared in Example 1.

Thus, adding a silicone adhesive to an acrylate adhesive (e.g., Durotak 87-2877) may be able to slow the flux rate of tetrabenazine.

### Example 4A. Preparation of Tetrabenazine in Adhesives with No Functional Groups

Batch Composition

| | | wet | solids | dry | |
|---|---|---|---|---|---|
| Ingredient | Function | grams | % | grams | dry % |
| Tetrabenazine | Active | 4.25 | | 4.25 | 7.1% |
| (+) (TBZ) | Ingredient | | | | |
| Ethyl acetate | Solvent | 10.00 | | | |
| Ethanol | Solvent | 5.00 | | | |
| DuroTak 87- | Adhesive | 100.0 | 43.70% | 43.70 | 72.9% |
| 900A | | | | | |
| Propyl Gallate | Antioxidant | 0.030 | | 0.030 | 0.050% |
| Plastoid B | Crystallization | 12.00 | | 12.000 | 20.0% |
| | Inhibitor/ | | | | |
| | solubilizer | | | | |
| total | | 119.25 | | 59.98 | 100.0% |

Plastoid B is a copolymer of butyl methacrylate and methyl methacrylate, manufactured by Evonik.

### Procedure for Preparation

The following procedure was followed for the preparation of the patch in Example 4A. In a 50-mL beaker, dissolve propyl gallate in ethanol by hand mixing. Separately, in a 250-mL beaker, add in Ethyl acetate, mix with a mechanical stirrer at low speed. Add in TBZ, and follow by Plastoid B powder while mixing. When Plastoid B dissolved, add in weighed DuroTak 87-900A while mixing. Mix, at moderate speed, for 30 min, or till homogeneous. Cast the solution, using a 10-mil coating applicator on 3M's backing film, Scotchpak 9723 film. Air dry the casting for 10 min, and oven dry at 85° C. for 10 min. Cover the dried adhesive with split Loparex release liners. Die cut the covered coating into 60 cm2 patches, using a steel rule die.

The patch has good skin adhesion and shear strength, and adheres snugly on skin for more than 48 hours.

The patch was die-cut to fix on the Franz cells for skin permeation study. The test results are reported in Example 6.

No crystals were observed on the patch for 4 weeks at 40° C., indicating good physical stability of the transdermal patch formulation. No degradation was observed on the patch for 4 weeks at 40° C., indicating good chemical stability of the transdermal patch formulation.

### Examples 4B and 4C. Preparation of Tetrabenazine in Adhesives with No Functional Groups

Two similar formulations were prepared following the same procedure as shown in Example 4A. These are given in the following table (dry composition):

| Formulation | Function | Ex 4B | Ex 4C |
|---|---|---|---|
| TBZ(+) | Active | 7.0% | 7.1% |
| DT 87-900A | Adhesive | 92.5% | 72.9% |
| Propyl gallate | Antioxidant | 0.05% | 0.05% |
| Soluplus | Crystal Inhibitor/solubilizer | | 20.0% |

The patches prepared in Examples 4B and 4C were also tested for skin permeation study.

### Example 5. Stability Studies of Tetrabenazine Patch Formulations

Various patch formulations of tetrabenazine were prepared and tested for chemical and/or physical stabilities. The inventors found that patch Formulations prepared with DuroTak 87-2287 or Duro-Tak 87-2677 Adhesives, which contains Functional Groups, showed yellowish color after shelf storage at 40° C. for 4 weeks, indicating instability of the active ingredient due to oxidation and/or other degradation. DuroTak 87-2287 has hydroxyl functional groups of —OH and epoxy, and DuroTak 87-2677 has acidic functional group of —COOH. In contrast, patch formulations prepared using DuroTak 87-900A, which do not have any functional groups, were found to be stable for 4 weeks at 40° C., see Example 4A.

The inventors also found that patch Formulations prepared without propyl gallate as antioxidants result in degradation of active ingredients. Impurities (drug-related) such as TBZ 01, TBZ 02, and TBZ 04 were formed and detected by HPLC, if no antioxidant is used. Antioxidants that can prevent oxidation and/or other degradation of TBZ include propyl gallate, citric acid, ascorbic acid, vitamin E (tocopherol acetate), etc.

Tetrabenazine and related compounds were analyzed using isocratic reversed phase HPLC with UV detector.

Column: Gemini C18, 4.6×150 mm, 5 μm particle size, or equivalent.
Column temperature: 45° C.

### Injection Volume: 10 μL

### Detection Wavelength: 210 nm

### Mobile Phase: Ratio of Mobile Phase A/Mobile Phase B=44:56

Mobile Phase A: 10 mM K₂HPO₄ in H₂O

### Mobile Phase B: Acetonitrile

Flow Rate: 1.2 mL/min

### Run Time: 12 min

Retention Time: about 5.5 min for tetrabenazine
Retention times for Impurity 1 (TBZ01) is at about 1.95 min; Impurity 2 (TBZ02) is at about 3.10 min; and Impurity 4 (TBZ04) is at about 5.29 min.

The inventors further found that without use of Soluplus or Plastoid B as crystallization inhibitor/solubilizer, see e.g., Example 4B, crystals appeared on the patch after shelf storage for 2 weeks at ambient temperature. Formation of crystals retards the skin permeation of the patch formulations.

Thus, preferred composition should contain crystallization inhibitors so that the active ingredient remains in the adhesive matrix in amorphous form for at least 12 months of storage at room temperature. Crystallization inhibitors preferred include:
PVP (polyvinylpyrrolidone) polymers: Kollidon K30 or K90F (manufactured by BASF), Plasdone K20/32 or Plasdone K90 (manufactured by Ashland Chemical).
Crosslink PVP polymers: Kollidon CL
PVP copolymers (copovidones): Plasdone S-630Copovidone (Asland)
Cellulose-based polymers: Hydroxylpropyl methyl cellulose (HPMC/Methocel), ethyl cellulose (Ethocel by Dow Chemica) e.g, Hydroxylpropyl cellulose (HPC, e.g. Klucel by Ashland)
Polycarboxylic acid polymers: Carbopol (manufactured by Lubrizol)
Polymethacrylates: Plastoid B, Eudragit E100, Eudragit L100-55 (manufactured by Evonik)
Soluplus (BASF): a polyethylene glycol, polyvinyl acetate and polyvinylcaprolactame-based graft copolymer (PVAc-PVCap-PEG)

### Example 6. Skin Permeation Studies

Patch formulations prepared in Examples 4A-4C were used for a skin permeation study using the following protocol:
Franz cell assembly—Logan Instruments (6-cell unit)
Each cell has 12 mL volume, 1.5 cm diameter orifice
Receptor medium was a phosphate buffer solution (PBS) pH 7.4
Cell temperature was maintained at 37° C.
Sampling method: took 1.5 mL for HPLC assay, emptied cell, replaced with fresh medium
Sampling time points: 2, 4, 8, 12, 24 and 48 hours
Cadaver skin was used and was obtained from New York Fighters Skin Bank.
Assay method for media: HPLC.

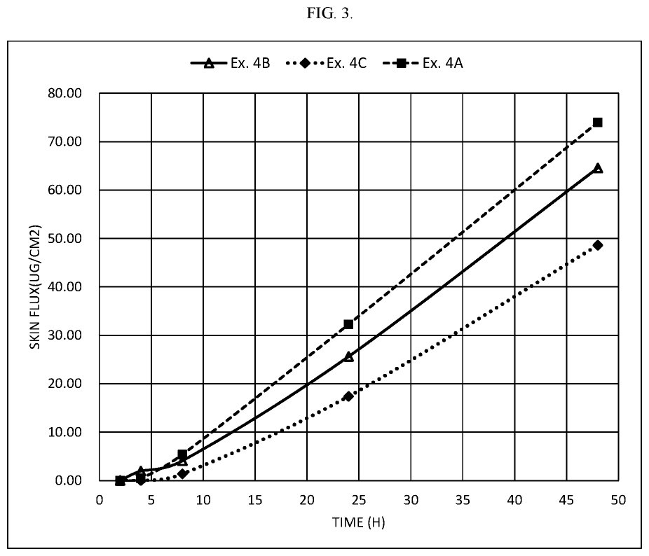

RESULTS of the study are presented in the table and the plot below. The values presented are cumulative amount of TBZ permeated per cm2 (i.e. μg/cm2). See also FIG. 3.

| AVERAGE | 2 h | 4 h | 8 h | 24 h | 48 h |
|---|---|---|---|---|---|
| Ex 4B | 0.00 | 1.95 | 4.07 | 25.61 | 64.56 |
| Ex 4C | 0.00 | 0.05 | 1.36 | 17.38 | 48.59 |
| Ex 4A | 0.00 | 0.47 | 5.38 | 32.27 | 73.99 |

### Example 7. In Vivo Pharmacokinetic Studies

The objective of this study was to evaluate the comparative bioavailability of a test TBZ patch, 8 mg/96 hr (see formulation of Example 4A) when applied for a single 96-hour application period relative to thrice-daily doses of the reference product, Tetrabenazine Tablets, 12.5 mg (Lupin) administered under fasted conditions from Day 1 through Day 4 (total dose of 150 mg) in healthy adult male subjects under fasted conditions.

This was an open-label, randomized, two-treatment, two-period, two-sequence, crossover study under fasted conditions comparing the test and reference products. The study was conducted with 16 healthy, non-tobacco-, non-nicotine-using, adult male subjects. In one period of the study, one (1) TBZ patch, 8 mg/96 hr was applied to the subject's upper outer left arm and kept in place for 96 hours following an overnight fast of at least 10 hours. In the other study period, 1×12.5 mg Tetrabenazine Tablet (Lupin) was administered every 8 hours from Day 1 through Day 4 for a total daily dose of 37.5 mg and a total dose of 150 mg for the 12 doses over the 4 days. For Treatment B (reference), the subjects conducted an overnight fast of at least 10 hours only prior to the 0-hour dose (Day 1). Subsequent doses were administered following a fast of at least 2 hours. The order of administration followed a two-sequence randomization schedule. Subjects were confined at the clinical facility from at least 10 hours before dosing (0-hour) until at least 120 hours after Day 1, 0-hour dosing in each study period. The interval between doses (0-hour) was 14 days.

Blood samples were collected pre-dose and at intervals over 120 hours after dosing (0-hour) with the study drug in each study period. Samples were analyzed by the bioanalytical laboratory for those subjects who were dosed with at least one of the study products (Treatment A or B).

The plasma concentrations of TBZ (RR and SS isomers) and its active metabolites (RRR, SSS, SRR and RSS) were measured by a fully validated analytical procedure. Statistical analysis using Analysis of Variance methodology was performed to evaluate the bioavailability of the test formulation relative to that of the reference product based on the plasma concentrations of these six analytes.

Study data were collected on source documents.

The study was designed based on the known pharmacokinetics of tetrabenazine tablets and its metabolites, recommendations in the FDA Draft Guidance on tetrabenazine tablets, and generally accepted standards for the conduct of bioavailability/bioequivalence studies under fasted conditions and adhesion studies.

To minimize any possibility of a carry-over effect, a washout period of at least 10 days was selected for this study.

### Treatment A

The active pharmaceutical ingredient is the RR stereoisomer of tetrabenazine. All patches (see formulation shown in Example 4A) were applied to the upper outer left arm, at least 2 inches below the shoulder and above the elbow. Patch was applied to the subject's upper outer left arm and kept in place for 96 hours following an overnight fast of at least 10 hours.

### Treatment B

The active pharmaceutical ingredient is the racemic form of two stereoisomers (RR and SS) of tetrabenazine. Administered every 8 hours from Day 1 through Day 4. The subjects conducted an overnight fast of at least 10 hours only prior to the 0-hour dose. Subsequent doses were administered following a fast of at least 2 hours. Total daily dose of the reference product was equal to 37.5 mg over three 8-hour dosing intervals for a total dose of 150 mg for the 12 doses over the 4 days.

The lower total dose of TBZ administered via the test patch (8 mg) as compared to the reference tablet (150 mg) was based on the premise that absorption of the biologically active RR isomer of TBZ from the active pharmaceutical ingredient would be more extensive from the patch as a result of reduced metabolism of TBZ to HTBZ from by-passing first-pass metabolism with topical administration. The active pharmaceutical ingredient in the tetrabenazine tablet is the racemic form (RR, SS) whereas the active pharmaceutical ingredient in the test TBZ patch is the RR isomer.

Adverse events were collected and tabulated. No formal statistical analyses were performed.

For Treatment A (test), 26 blood samples were collected from each subject during the respective period of the study: pre-dose within 60 minutes before patch application (0-hour) and 0.5, 1.0, 2.0, 4.0, 6.0, 8.0, 12.0, 16.0, 24.0, 32.0, 40.0, 48.0, 56.0, 64.0, 72.0, 80.0, 88.0, 96.0 (within 5 minutes before patch removal), 97.0, 98.0, 100.0, 104.0, 108.0, 114.0 and 120.0 hours post-dose. All times are relative to the dosing minute (time of patch application). For Treatment B (reference), 51 blood samples were collected from each subject during the respective period of the study: pre-dose within 60 minutes before dosing (Day 1, 0-hour) and 0.17, 0.33, 0.5, 0.75, 1.0, 1.33, 1.67, 2.0, 2.5, 3.0, 4.0, 6.0, 8.0^{†}, 8.33, 8.67, 9.0, 9.5, 10.0, 12.0, 16.0^{†}, 16.5, 17.0, 18.0, 20.0, 24.0^{†}, 25.0, 26.0, 28.0, 32.0^{†}, 34.0, 36.0, 40.0^{†}, 44.0, 48.0^{†}, 52.0, 56.0^{†}, 60.0, 64.0^{†}, 68.0, 72.0^{†}, 76.0, 80.0^{†}, 84.0, 88.0^{†}, 92.0, 96.0, 100.0, 104.0, 112.0 and 120.0 hours post-dose. All times are relative to the dosing minute (Day 1, 0-hour). ^{†}Samples collected immediately before each dosing with an allowed deviation of −5 minutes to accommodate dosing activities, as necessary.

The plasma concentrations of TBZ (RR and SS isomers) and its HTBZ metabolites (RRR, SSS, SRR and RSS) were measured by a fully validated analytical procedure for those subjects who were dosed with at least one of the study products (Treatment A or B). Concentrations of total TBZ were expressed in molar units as the sum of molar concentrations of TBZ+RRR+SSS+SRR+RSS.

The following PK parameters were evaluated for each of the six analytes (two TBZ isomers [RR and SS] and the four HTBZ metabolites [RRR, SSS, SRR and RSS]), TBZ combined (RR+SS), and total TBZ: AUC₀₋ₜ, AUC₀₋₉₆, AUC_{0-∞}, Cₘₐₓ, Tₘₐₓ, Kₑₗ and T_{1/2}. Additionally, the metabolic ratio (AUC₀₋ₜ(met)/AUC_{0-t(TBZ)}) for each of the four HTBZ metabolites (RRR, SSS, SRR and RSS) was evaluated as well as the apparent clearance (CL/F) of TBZ.

For each of the six analytes, TBZ combined (RR+SS), and total TBZ, an Analyses of Variance was performed on In-transformed Cₘₐₓ, AUC₀₋₉₆ and AUC_{0-∞} using the General Linear Model (GLM) procedure of SAS® (Version 9.4). The statistical model contained main effects of sequence, subject-within-sequence, treatment, and period.

Confidence intervals (90%) on the geometric mean ratios (obtained from logarithmic transformed data) for AUC₀₋₉₆, AUC_{0-∞} and Cₘₐₓ for the comparison of the test formulation to the reference product were constructed to test two one-sided hypotheses at the α=0.05 level of significance. Similar analyses were performed for the additional PK parameters: metabolic ratio for the four metabolites and CL/F for TBZ. Descriptive statistics are reported for all PK parameters by treatment.

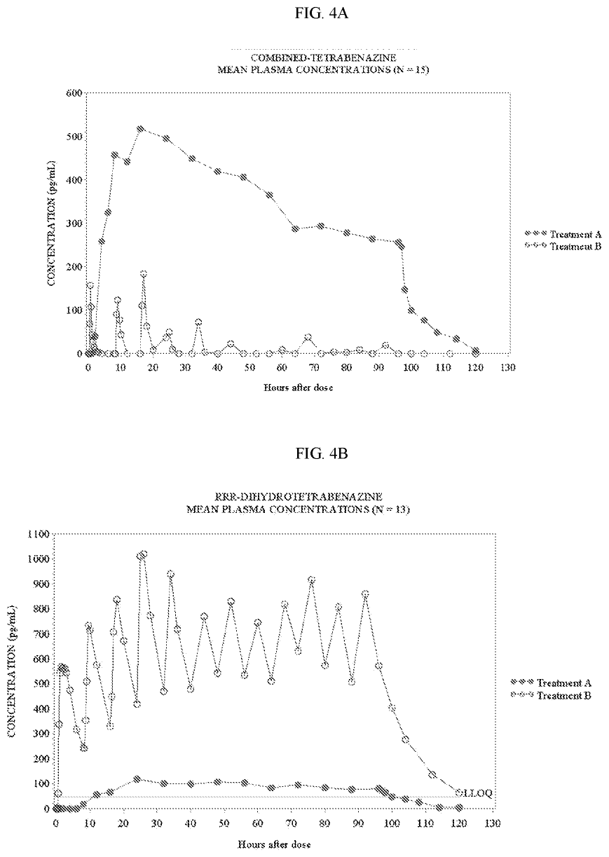

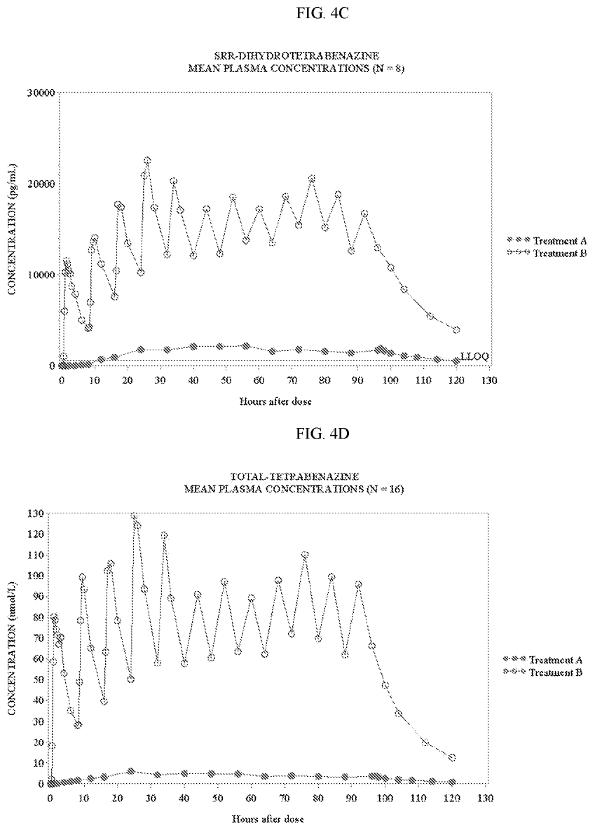

SUMMARY OF RESULTS: Mean plasma concentration versus time plots (linear) are presented below as FIG. 4A for TBZ (RR and SS isomers combined), as FIG. 4B for RRR HTBZ, as FIG. 4C for SRR HTBZ, and as FIG. 4D for total TBZ. Arithmetic means of the pharmacokinetic parameters (untransformed) are provided in Table 1A for Treatment A and in Table 1B for Treatment B. All available data are presented.

Geometric means, ratio of geometric means, and their associated 90% confidence intervals and intra-subject CV (ISCV %) values based on ANOVA (ln-transformed) are provided in Table 2A for TBZ (RR and SS isomers combined), in Table 2B for RRR HTBZ, in Table 2C for SRR HTBZ, and in Table 2D for total TBZ.

TABLE 1A
Mean Pharmacokinetic Parameter Values by Analyte for Treatment A (Single Dose of 8 mg/96 hr patch)

| | AUC₀₋ₜ | AUC_{0-∞} | AUC₀₋₉₆ | Cₘₐₓ | Tₘₐₓ | Kₑₗ | T_{1/2} | CL/F_{topical} | AUC_{0-∞(met)}/ | AUC_{0-∞(met)}/ |
|---|---|---|---|---|---|---|---|---|---|---|
| Analyte | (h · pg/mL) | (h · pg/mL) | (h · pg/mL) | (pg/mL) | (h)* | (h⁻¹) | (h) | (L/h) | AUC_{0-∞(TBZ)} | AUC_{0-∞(TBZ)} |
| TBZ (RR +SS) | 35060.5095 | 37527.1500 | 33594.8216 | 570.1400 | 20.0083 | 0.1039 | 8.4713 | 255.8089 | NA | NA |
| | (n = 16) | (n = 14) | (n = 16) | (n = 16) | (n = 16) | (n = 14) | (n = 14) | (n = 14) | | |
| RRTBZ | 35060.5095 | 37527.1500 | 33594.8216 | 570.1400 | 20.0083 | 0.1039 | 8.4713 | 255.8089 | NA | NA |
| | (n = 16) | (n = 14) | (n = 16) | (n = 16) | (n = 16) | (n = 14) | (n = 14) | (n = 14) | | |
| SS TBZ^{†} | NE | NE | NE | NE | NE | NE | NE | NE | NA | NA |
| RRR HTBZ | 8373.0650 | 13575.7690 | 7798.4167 | 126.1685 | 32.0000 | 0.0689 | 10.0542 | NA | 0.2256 | 0.2221 |
| | (n = 13) | (n = 1) | (n = 13) | (n = 13) | (n = 13) | (n = l) | (n = l) | | (n = l) | (n = 13) |
| SSS HTBZ^{†} | NE | NE | NE | NE | NE | NE | NE | NA | NE | NE |
| SRR HTBZ | 168212.4133 | 132862.9692 | 145012.7993 | 2424.4751 | 48.0000 | 0.0548 | 12.6436 | NA | 6.1003 | 3.7720 |
| | (n = 8) | (n = 1) | (n = 8) | (n = 8) | (n = 8) | (n = l) | (n = l) | | (n = l) | (n = 8) |
| RSS HTBZ^{†} | NE | NE | NE | NE | NE | NE | NE | NA | NE | NE |
| Total TBZ^{‡} | 400.4585 | 480.6111 | 356.3363 | 5.8772 | 28.0000 | 0.1199 | 8.0249 | NA | NA | NA |
| | (n = 16) | (n = 14) | (n = 16) | (n = 16) | (n = 16) | (n = 14) | (n = 14) | | | |

*Median
^{†}All concentrations for all subjects are BLQ (i.e., <LLOQ of 498.154 pg/mL for SS TBZ, 628.530 pg/mL for SSS HTBZ, and 499.875 pg/mL for RSS HTBZ)
^{‡}Units for AUC are h · nmol/L and units for Cmax are nmol/L
NA: Not Applicable
NE: Not Estimated

TABLE 1B
Mean Pharmacokinetic Parameter Values by Analyte for Treatment B (1 × 12.5 mg tablet every 8 hr for 4 days)

| | AUC₀₋ₜ | AUC_{0-∞} | AUC₀₋₉₆ | Cₘₐₓ | Tₘₐₓ | Kₑₗ | T_{1/2} | CL/F_{topical} | AUC_{0-∞(met)}/ | AUC_{0-∞(met)}/ |
|---|---|---|---|---|---|---|---|---|---|---|
| Analyte | (h · pg/mL) | (h · pg/mL) | (h · pg/mL) | (pg/mL) | (h)* | (h⁻¹) | (h) | (L/h) | AUC_{0-∞(TBZ)} | AUC_{0-∞(TBZ)} |
| TBZ (RR + SS)^{§} | 1164.6280 | NE | 1265.8975 | 294.2203 | 16.5000 | NE | NE | NE | NA | NA |
| | (n = 15) | | (n = 15) | (n = 15) | (n = 15) | | | | | |
| RR TBZ^{†} | 999.4895 | NE | 1100.7590 | 137.8475 | 16.5000 | NE | NE | NE | NA | NA |
| | (n = 15) | | (n = 15) | (n = 15) | (n = 15) | | | | | |
| SS TBZ^{‡} | 906.5096 | NE | 1189.0961 | 810.8560 | 9.0000 | NE | NE | NE | NA | NA |
| | (n = l) | | (n = l) | (n = l) | (n = l) | | | | | |
| RRR HTBZ | 62781.1003 | 64341.3948 | 57848.9411 | 1109.4245 | 25.0000 | 0.1068 | 7.4903 | NA | NE | 136.1972 |
| | (n = 16) | (n = 16) | (n = 16) | (n = 16) | (n = 16) | (n = 16) | (n = 16) | | | (n = 15) |
| SSS HTBZ | 1278309.0449 | 1318201.2129 | 1161623.5993 | 24946.1819 | 25.0000 | 0.0976 | 8.3115 | NA | NE | 2797.1341 |
| | (n = 16) | (n = 16) | (n = 16) | (n = 16) | (n = 16) | (n = 16) | (n = 16) | | | (n = 15) |
| SRR HTBZ | 922405.6631 | 1443897.0709 | 831246.5949 | 16560.4164 | 25.5000 | 0.1339 | 6.9547 | NA | NE | 1916.0880 |
| | (n = 16) | (n = 10) | (n = 16) | (n = 16) | (n = 16) | (n = 10) | (n = 10) | | | (n = 15) |
| RSS HTBZ | 351674.6037 | 375318.8572 | 336442.9774 | 7166.2672 | 26.0000 | 0.1288 | 5.8498 | NA | NE | 728.9960 |
| | (n = 16) | (n = 15) | (n = 16) | (n = 16) | (n = 16) | (n = 15) | (n = 15) | | | (n = 15) |
| Total TBZ^{¶} | 8204.8147 | 8465.2813 | 7477.0546 | 144.9376 | 25.0000 | 0.1209 | 7.1470 | NA | NA | NA |
| | (n = 16) | (n = 16) | (n = 16) | (n = 16) | (n = 16) | (n = 16) | (n = 16) | | | |

*Median
^{†}Most concentrations for all subjects are BLQ. One subject (# 3012) does not have at least 4 concentrations > LLOQ of 48.050 pg/mL.
^{‡}12 of 16 subjects have all BLQ concentrations. Three of 16 subjects have 1 to 3 concentrations and one subject (# 3013) has 5 concentrations > LLOQ of 498.154 pg/mL.
^{§}Most concentrations for all subjects are BLQ. One subject (# 3012) does not have at least 4 concentrations > LLOQ for either RR or SS TBZ when combined.
^{¶}Units for AUC are h-nmol/L and units for Cmax are nmol/L
NA: Not Applicable
NE: Not Estimated

TABLE 2A
Summary of Primary Endpoints Based on Plasma TBZ (RR and SS isomers combined) Concentration

| | | LS | | LSGM | 90% | | | | 90% CI |
|---|---|---|---|---|---|---|---|---|---|
| | | Geometric | Contrast | Ratio | Confidence | | P-value | P-value | within 80- |
| Parameter | Trt. | Mean | (# subjects) | (%) | Interval (%) | ISCV (%) | Period | Sequence | 125% |
| AUC₀₋₉₆ | A | 32375.579 | A vs B | 3806.18 | 2257.16-6418.27 | 95.7 | 0.4628 | 0.9175 | No |
| (h · pg/mL) | | | (n = 15) | | | | | | |
| | B | 850.605 | | | | | | | |
| Cₘₐₓ | A | 555.313 | A vs B | 341.20 | 209.67-555.23 | 87.1 | 0.4548 | 0.8874 | No |
| (pg/mL) | | | (n = 15) | | | | | | |
| | B | 162.754 | | | | | | | |

TABLE 2B
Summary of Primary Endpoints Based on Plasma RRR HTBZ Concentrations

| | | LS | | LSGM | 90% | | | | 90% CI |
|---|---|---|---|---|---|---|---|---|---|
| | | Geometric | Contrast | Ratio | Confidence | | P-value | P-value | within 80- |
| Parameter | Trt. | Mean | (# subjects) | (%) | Interval (%) | ISCV (%) | Period | Sequence | 125% |
| AUC₀₋₉₆ | A | 7164.062 | A vs B | 13.09 | 10.29-16.66 | 35.1 | 0.8685 | 0.7138 | No |
| (h · pg/mL) | | | (n = 13) | | | | | | |
| | B | 54726.280 | | | | | | | |
| Cₘₐₓ | A | 120.830 | A vs B | 11.17 | 9.30-13.41 | 26.4 | 0.7710 | 0.8794 | No |
| (pg/mL) | | | (n = 13) | | | | | | |
| | B | 1081.891 | | | | | | | |
| AUC_{0-t(RRR)}/ | A | 0.198 | A vs B | 0.22 | 0.13-0.37 | 82.5 | 0.8890 | 0.8039 | No |
| AUC_{0-t(TBZ)} | | | (n = 13) | | | | | | |
| | B | 89.628 | | | | | | | |

TABLE 2C
Summary of Primary Endpoints Based on Plasma SRR HTBZ Concentrations

| | | LS | | LSGM | 90% | | | | 90% CI |
|---|---|---|---|---|---|---|---|---|---|
| | | Geometric | Contrast | Ratio | Confidence | | P-value | P-value | within 80- |
| Parameter | Trt. | Mean | (# subjects) | (%) | Interval (%) | ISCV (%) | Period | Sequence | 125% |
| AUC₀₋₉₆ | A | 89903.262 | A vs. B | 12.36 | 6.22-24.54 | 80.4 | 0.2669 | 0.7158 | No |
| (h · pg/mL) | | | (n = 8) | | | | | | |
| | B | 727441.779 | | | | | | | |
| Cₘₐₓ | A | 1663.555 | A vs. B | 9.66 | 5.75-16.24 | 57.5 | 0.7489 | 0.6576 | No |
| (pg/mL) | | | (n = 8) | | | | | | |
| | B | 17218.960 | | | | | | | |
| AUC_{0-t(SRR)}/ | A | 2.395 | A vs. B | 0.22 | 0.08-0.58 | 135.7 | 0.8553 | 0.8952 | No |
| AUC_{0-t(TBZ)} | | | (n = 8) | | | | | | |
| | B | 1111.324 | | | | | | | |

TABLE 2D
Summary of Primary Endpoints Based on Plasma Total TBZ Concentrations

| | | LS | | LSGM | 90% | | | | 90% CI |
|---|---|---|---|---|---|---|---|---|---|
| | | Geometric | Contrast | Ratio | Confidence | | P-value | P-value | within 80- |
| Parameter | Trt. | Mean | (# subjects) | (%) | Interval (%) | ISCV (%) | Period | Sequence | 125% |
| AUC₀₋₉₆ | A | 203.527 | A vs B | 3.41 | 2.39-4.87 | 62.2 | 0.7135 | 0.6315 | No |
| (h · nmol/L) | | | (n = 16) | | | | | | |
| | B | 5969.194 | | | | | | | |
| AUC_{0-∞} | A | 259.219 | A vs B | 3.93 | 2.77-5.58 | 55.7 | 0.9446 | 0.6494 | No |
| (h · nmol/L) | | | (n = 14) | | | | | | |
| | B | 6597.700 | | | | | | | |
| Cmax | A | 3.534 | A vs B | 2.79 | 2.00-3.89 | 57.5 | 0.2807 | 0.6284 | No |
| (nmol/L) | | | (n = 16) | | | | | | |
| | B | 126.826 | | | | | | | |

Following application of the patch in Treatment A, TBZ concentrations of the RR isomer increased to a mean peak exposure (Cₘₐₓ) of 570 pg/mL at a median Tₘₐₓ of 20 hours and slowly decreased thereafter until removal of the patch at 96 hours, after which time concentrations declined with an average terminal half-life of 8.5 hours. All concentrations of the SS isomer of TBZ were BLQ. Two of the four HTBZ metabolites were detected (RRR and SRR diastereomers), with the SRR isomer approximately 20-fold higher in peak (Cₘₐₓ) and total (AUC₀₋ₜ) plasma exposures.

Following thrice-daily doses of the tablet over 96 hours in Treatment B, most concentrations of RR and SS for all subjects were BLQ. For those RR concentrations that were above the LLOQ of 48.050 pg/mL, the mean Cₘₐₓ was 138 pg/mL and median Tₘₐₓ was 16.5 hours following the first dose or 0.5 hr after the third dose on Day 1 All four active HTBZ metabolites were detected, with the SSS isomer predominating based on the highest mean AUC and Cₘₐₓ values, followed in rank order by SRR, RSS and RRR. No metabolic AUC ratios (AUC_{0-∞(met)}/AUC_{0-∞(TBZ)}) were calculated owing to lack of AUC_{0-∞(TBZ)} data for RR, SS and RR+SS analytes, but estimates were made using AUC₀₋ₜ₍ₘₑₜ₎/AUC_{0-t(TBZ)}) data instead.

Comparing Treatments A (patch) and B (tablet), the extent of TBZ metabolism to HTBZ is less for Treatment A as indicated by the approximate 500-fold smaller least-squares geometric mean (LSGM) AUC₀₋ₜ₍ₘₑₜ₎/AUC_{0-t(TBZ)}) ratios for RRR and SRR. Likewise, the RRR and SRR AUC₀₋₉₆ and Cₘₐₓ values for Treatment A (uncorrected for dose difference) are 8- to 10-fold lower than those values for Treatment B, as demonstrated by LSGM A/B ratios of approximately 10-13% for the RRR and SRR parameters. The LSGMs of RR+SS AUC₀₋₉₆ and Cₘₐₓ for Treatment A are about 38-fold and 3.4-fold higher than those for Treatment B, respectively. LSGM A/B ratios for total TBZ AUC₀₋₉₆, AUC_{0-∞} and Cₘₐₓ are low at 3-4%.

CONCLUSION: The TBZ patch, 8 mg/96 hr applied for a single 96-hour application provided higher concentrations of RR-tetrabenazine, with no inter-conversion to the SS isomer, and less extent of metabolism to the active HTBZ isomers with a lower dose compared to thrice-daily doses of TBZ tablets administered under fasted conditions from Day 1 through Day 4 (total dose of 150 mg) in healthy male subjects.

No patches (Treatment A [test]) were removed during the study because of irritation reactions. There were no serious adverse events reported during the study, and no subjects were discontinued because of adverse events.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A method of treating a hyperkinetic movement disorder in a subject in need thereof, the method comprising administering to the subject a pharmaceutical composition comprising an active ingredient comprising R,R-tetrabenazine, wherein the administering bypasses first-pass metabolism and delivers, e.g., continuously or substantially continuously delivers, to the subject about 0.1 mg/day to about 20 mg/day of R,R-tetrabenazine, e.g., at a substantially constant rate, preferably, about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine.

2. The method of claim 1, wherein the administering comprises applying the pharmaceutical composition to the skin of the subject to transdermally deliver to the subject about 0.1 mg/day to about 20 mg/day of R,R-tetrabenazine, preferably, about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine.

3. The method of claim 1 or 2, wherein the pharmaceutical composition comprises an adhesive composition, e.g., in a transdermal delivery device, which comprises the active ingredient dispersed in an adhesive (preferably a pressure sensitive adhesive), wherein the adhesive composition is applied to the subject to deliver about 0.1 mg/day to about 20 mg/day of R,R-tetrabenazine at a substantially constant rate for up to 24 hours post application, up to 48 hours post application, up to 96 hours post application, or up to 1 week post application.

4. The method of claim 3, wherein the adhesive composition comprises the active ingredient dispersed in a non-reactive acrylate pressure sensitive adhesive, preferably, the active ingredient is in an amount of about 1% to about 20%, such as about 2% to about 7% by weight and the non-reactive acrylate pressure sensitive adhesive is in an amount of about 50% to about 97% by weight.

5. The method of claim 3 or 4, wherein the adhesive composition further comprises a gallate antioxidant such as propyl gallate.

6. The method of any one of claims 3-5, wherein the adhesive composition further comprises a crystallization inhibitor in an amount effective to prevent formation of drug crystals after shelf storage for two weeks at ambient temperature.

7. The method of any one of claims 3-6, wherein the adhesive composition further comprises a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof.

8. The method of any one of claims 1-7, wherein the sole active ingredient in the pharmaceutical composition is a substantially pure R,R-isomer of tetrabenazine.

9. The method of any one of claims 1-8, wherein the hyperkinetic movement disorder is selected from Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.

10. The method of any one of claims 1-9, wherein the administering is conducted without regard to the fed status of the subject.

11. The method of any one of claims 1-10, wherein the subject is an extensive metabolizer.

12. A method of treating a hyperkinetic movement disorder in a subject in need thereof, the method comprising administering to the subject a pharmaceutical composition comprising an active ingredient comprising R,R-tetrabenazine, wherein the administering provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ, Cₘₐₓ of R,R-tetrabenazine/(Cₘₐₓ of R,R,R-HTBZ+Cₘₐₓ of S,R,R-HTBZ), ranges from about 1:1 to about 1:5, or the ratio of the steady state plasma concentration of R,R-tetrabenazine to the combined steady state plasma concentration of R,R,R-HTBZ and S,R,R-HTBZ, Cₛₛ of R,R-tetrabenazine/(Cₛₛ of R,R,R-HTBZ+Cₛₛ of S,R,R-HTBZ), ranges from about 1:1 to about 1:5, preferably, the ratio of maximum plasma concentration or steady state plasma concentration of R,R,R-HTBZ to S,R,R-HTBZ ranges from about 1:5 to about 1:30 (e.g., about 1:10 to about 1:20), for example, the ratio of maximum plasma concentration (Cₘₐₓ) or steady state plasma concentration (Cₛₛ) of R,R-tetrabenazine:R,R,R-HTBZ:S,R,R-HTBZ is about 17-40:3-10:50-80, preferably, the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75, such as about 0.15 to about 0.5, preferably, the administering provides no detectable S,S-tetrabenazine, R,S,S-HTBZ, or S,S,S-HTBZ.

13. The method of claim 12, wherein the administering bypasses first-pass metabolism and delivers to the subject R,R-tetrabenazine continuously or substantially continuously, e.g., at a substantially constant rate.

14. The method of claim 12 or 13, wherein the pharmaceutical composition is administered transdermally, intravenously, subcutaneously, intramuscularly, or via a depot.

15. The method of claim 12 or 13, wherein the pharmaceutical composition is administered transdermally.

16. The method of any one of claims 12-15, wherein the pharmaceutical composition is administered once a day, once in more than a day, such as once in two days, once in three days, once in four days, once in five days, once in six days, once in a week, or once in more than a week.

17. The method of any one of claims 12-16, wherein the pharmaceutical composition is formulated to provide a dose of about 2 mg/day of R,R-tetrabenazine for 4 days when administered once, and the method comprises administering the pharmaceutical composition to provide a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ S,R,R-HTBZ later than the mean Tₘₐₓ of R,R-tetrabenazine;
c) a mean Cₘₐₓ of R,R-tetrabenazine of about 300 pg/ml to about 700 pg/ml;
d) a mean Cₘₐₓ of R,R,R-HTBZ of about 60 pg/ml to about 200 pg/ml;
e) a mean Cₘₐₓ of S,R,R-HTBZ of about 1000 pg/ml to about 3000 pg/ml;
f) a mean AUC₀₋₉₆ of R,R-tetrabenazine of about 20 ng*h/ml to about 50 ng*h/ml;
g) a mean AUC₀₋₉₆ of R,R,R-HTBZ of about 4 ng*h/ml to about 12 ng*h/ml; and/or
h) a mean AUC₀₋₉₆ of S,R,R-HTBZ of about 70 ng*h/ml to about 200 ng*h/ml.

18. The method of any one of claims 12-16, wherein the pharmaceutical composition is formulated to provide a dose of about 4-6 mg/day of R,R-tetrabenazine for at least one day, for example, two days, three days, four days, or a week, when administered once, and the method comprises administering the pharmaceutical composition to provide a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 600 pg/ml to about 2100 pg/ml;
d) a mean Cₘₐₓ of R,R,R-HTBZ of about 120 pg/ml to about 600 pg/ml;
e) a mean Cₘₐₓ of S,R,R-HTBZ of about 2000 pg/ml to about 9000 pg/ml;
f) a mean AUC₀₋₉₆ of R,R-tetrabenazine of about 40 ng*h/ml to about 150 ng*h/ml;
g) a mean AUC₀₋₉₆ of R,R,R-HTBZ of about 8 ng*h/ml to about 36 ng*h/ml; and/or
h) a mean AUC₀₋₉₆ of S,R,R-HTBZ of about 140 ng*h/ml to about 600 ng*h/ml.

19. The method of any one of claims 12-16, wherein the pharmaceutical composition is formulated to provide a dose of about 1-10 mg/day of R,R-tetrabenazine for at least one day, for example, two days, three days, four days, or a week, when administered once, and the method comprises administering the pharmaceutical composition to provide a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 150 pg/ml to about 3500 pg/ml;
d) a mean Cₘₐₓ of R,R,R-HTBZ of about 30 pg/ml to about 1000 pg/ml;
e) a mean Cₘₐₓ of S,R,R-HTBZ of about 500 pg/ml to about 15 ng/ml;
f) a mean AUC₀₋₉₆ of R,R-tetrabenazine of about 10 ng*h/ml to about 250 ng*h/ml;
g) a mean AUC₀₋₉₆ of R,R,R-HTBZ of about 2 ng*h/ml to about 60 ng*h/ml; and/or
h) a mean AUC₀₋₉₆ of S,R,R-HTBZ of about 35 ng*h/ml to about 1000 ng*h/ml.

20. The method of any one of claims 12-19, wherein the administering provides a pharmacokinetic profile in the subject characterized by that (1) during a first time period, a maximum plasma concentration of R,R-tetrabenazine of about 150 pg/ml to about 3500 pg/ml is reached for the first period of time at a first time point, wherein the first period of time is from the time of initial administration to about 24 hours thereafter; and optionally (2) after the first time point, the plasma concentration of R,R-tetrabenazine remains substantially constant for a sustained period of about 24 hours, about 48 hours, about 72 hours, about 96 hours, or more, and preferably, the average terminal half-life of R,R-tetrabenazine is about 8.5 hours±40% CV.

21. The method of claim 20, wherein the plasma concentration of R,R-tetrabenazine at 24 hours post the first time point is about 50% to about 200%, for example, about 75% to about 150% of that at the first time point.

22. The method of any one of claims 12-21, wherein the pharmaceutical composition comprises an adhesive composition, e.g., in transdermal delivery device, which comprises the active ingredient dispersed in an adhesive (preferably a pressure sensitive adhesive).

23. The method of claim 22, wherein the adhesive composition comprises the active ingredient dispersed in a non-reactive acrylate pressure sensitive adhesive, preferably, the active ingredient is in an amount of about 1% to about 20% such as about 2% to about 7% by weight and the non-reactive acrylate pressure sensitive adhesive is in an amount of about 50% to about 97% by weight.

24. The method of claim 22 or 23, wherein the adhesive composition further comprises a gallate antioxidant such as propyl gallate.

25. The method of any one of claims 22-24, wherein the adhesive composition further comprises a crystallization inhibitor in an amount effective to prevent formation of drug crystals after shelf storage for two weeks at ambient temperature.

26. The method of any one of claims 22-25, wherein the adhesive composition further comprises a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof.

27. The method of any one of claims 12-26, wherein the sole active ingredient in the pharmaceutical composition is a substantially pure R,R-isomer of tetrabenazine.

28. The method of any one of claims 12-27, wherein the hyperkinetic movement disorder is selected from Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.

29. The method of any one of claims 12-28, wherein the administering is conducted without regard to the fed status of the subject.

30. The method of any one of claims 12-29, wherein the subject is an extensive metabolizer.

31. A method of treating a hyperkinetic movement disorder in a subject in need thereof, the method comprising applying to the subject a transdermal delivery device, preferably, a transdermal delivery device comprising a drug-in-adhesive layer, wherein the drug-in-adhesive layer comprises (1) an active ingredient comprising R,R-tetrabenazine, (2) a non-reactive acrylate pressure sensitive adhesive; (3) an optional crystallization inhibitor; and (4) an optional antioxidant, wherein the transdermal delivery device provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ, Cₘₐₓ of R,R-tetrabenazine/(Cₘₐₓ of R,R,R-HTBZ+Cₘₐₓ of S,R,R-HTBZ), ranges from about 1:1 to about 1:5, or the ratio of the steady state plasma concentration of R,R-tetrabenazine to the combined steady state plasma concentration of R,R,R-HTBZ and S,R,R-HTBZ, Cₛₛ of R,R-tetrabenazine/(Cₛₛ of R,R,R-HTBZ+Cₛₛ of S,R,R-HTBZ), ranges from about 1:1 to about 1:5, preferably, the ratio of maximum plasma concentration or steady state plasma concentration of R,R,R-HTBZ to S,R,R-HTBZ ranges from about 1:5 to about 1:30 (e.g., about 1:10 to about 1:20), for example, the ratio of maximum plasma concentration or steady state plasma concentration of R,R-tetrabenazine:R,R,R-HTBZ:S,R,R-HTBZ is about 17-40:3-10:50-80, preferably, the ratio of AUC_{0-∞} of SRR-HTBZ to the AUC_{0-∞} of R,R-tetrabenazine is about 1 to about 15, such as about 1.5 to about 11, and/or the ratio of AUC_{0-∞} of RRR-HTBZ to the AUC_{0-∞} of R,R-tetrabenazine is about 0.1 to about 0.75 such as about 0.15 to about 0.5, preferably, the administering provides no detectable S,S-tetrabenazine, R,S,S-HTBZ, or S,S,S-HTBZ.

32. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising R,R-tetrabenazine, (2) a non-reactive acrylate pressure sensitive adhesive; (3) an optional crystallization inhibitor; and (4) an optional antioxidant, wherein the transdermal delivery device is applied to transdermally deliver to the subject about 0.1 mg/day to about 20 mg/day of R,R-tetrabenazine, preferably, about 0.5 mg/day to about 10 mg/day of R,R-tetrabenazine, about 0.5 mg/day to about 8 mg/day of R,R-tetrabenazine, or about 2 mg/day to about 6 mg/day of R,R-tetrabenazine.

33. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising R,R-tetrabenazine, (2) a non-reactive acrylate pressure sensitive adhesive; (3) an optional crystallization inhibitor; and (4) an optional antioxidant, wherein the transdermal delivery device is applied to the subject to achieve a therapeutically effective plasma concentration of R,R-tetrabenazine, R,R,R-dihydrotetrabenazine (HTBZ) and S,R,R-HTBZ for at least 6 hours, or at least 12 hours, preferably, at least 24 hours.

34. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising R,R-tetrabenazine, (2) a non-reactive acrylate pressure sensitive adhesive; (3) an optional crystallization inhibitor; and (4) an optional antioxidant, wherein the transdermal delivery device is applied to the subject to achieve a substantially constant steady state plasma concentration of R,R-tetrabenazine above 150 pg/ml, for a sustained period of at least 6 hours, or at least 12 hours, preferably, at least 24 hours.

35. A method of treating a hyperkinetic movement disorder in a subject in need thereof, the method comprising applying to the subject a transdermal delivery device, preferably, a transdermal delivery device comprising a drug-in-adhesive layer, wherein the drug-in-adhesive layer comprises (1) an active ingredient comprising a deuterated R,R-tetrabenazine, (2) a non-reactive acrylate pressure sensitive adhesive; (3) an optional crystallization inhibitor; and (4) an optional antioxidant, wherein the transdermal delivery device provides a therapeutically effective plasma concentration of deuterated R,R-tetrabenazine, the dihydrotetrabenazine metabolite of the deuterated R,R-tetrabenazine, deuterated R,R,R-HTBZ and deuterated S,R,R-HTBZ, wherein, the ratio of the maximum plasma concentration of the deuterated R,R-tetrabenazine to the combined maximum plasma concentration of the deuterated R,R,R-HTBZ and deuterated S,R,R-HTBZ ranges from about 1:1 to about 1:7.5, or the ratio of the steady state plasma concentration of the deuterated R,R-tetrabenazine to the combined steady state plasma concentration of the deuterated R,R,R-HTBZ and deuterated S,R,R-HTBZ ranges from about 1:1 to about 1:7.5, preferably, the ratio of maximum plasma concentration or steady state plasma concentration of the deuterated R,R,R-HTBZ to deuterated S,R,R-HTBZ ranges from about 1:5 to about 1:30 (e.g., about 1:10 to about 1:20), preferably, the administering provides no detectable deuterated S,S-tetrabenazine, deuterated R,S,S-HTBZ, or deuterated S,S,S-HTBZ.

36. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising a deuterated R,R-tetrabenazine, (2) a non-reactive acrylate pressure sensitive adhesive; (3) an optional crystallization inhibitor; and (4) an optional antioxidant, wherein the transdermal delivery device is applied to transdermally deliver to the subject about 0.1 mg/day to about 20 mg/day of deuterated R,R-tetrabenazine, preferably, about 0.5 mg/day to about 10 mg/day of deuterated R,R-tetrabenazine, about 0.5 mg/day to about 8 mg/day of deuterated R,R-tetrabenazine, or about 2 mg/day to about 6 mg/day of deuterated R,R-tetrabenazine.

37. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising deuterated R,R-tetrabenazine, (2) a non-reactive acrylate pressure sensitive adhesive; (3) an optional crystallization inhibitor; and (4) an optional antioxidant, wherein the transdermal delivery device is applied to the subject to achieve a therapeutically effective plasma concentration of deuterated R,R-tetrabenazine, the dihydrotetrabenazine metabolite of the deuterated R,R-tetrabenazine, deuterated R,R,R-HTBZ and deuterated S,R,R-HTBZ, for at least 6 hours, or at least 12 hours, preferably, at least 24 hours.

38. A method of treating a hyperkinetic movement disorder in a subject in need thereof, comprising applying to the subject a transdermal delivery device, preferably, a transdermal delivery device comprising a drug-in-adhesive layer, wherein the drug-in-adhesive layer comprises (1) an active ingredient comprising a deuterated R,R-tetrabenazine, (2) a non-reactive acrylate pressure sensitive adhesive; (3) an optional crystallization inhibitor; and (4) an optional antioxidant, wherein the transdermal delivery device is applied to the subject to achieve a substantially constant steady state plasma concentration of the deuterated R,R-tetrabenazine above 150 pg/ml, for a sustained period of at least 6 hours, or at least 12 hours, preferably, at least 24 hours.

39. The method of any one of claims 31-38, wherein the transdermal delivery device is applied once a day, once in more than a day, such as once in two days, once in three days, once in four days, once in five days, once in six days, once in a week, or once in more than a week.

40. The method of any one of claims 31-39, wherein the transdermal delivery device comprises a drug-in-adhesive layer, and the drug-in-adhesive layer comprises the active ingredient dispersed in the non-reactive acrylate pressure sensitive adhesive, preferably, the active ingredient is in an amount of about 1% to about 20%, such as about 2% to about 7% by weight and the non-reactive acrylate pressure sensitive adhesive is in an amount of about 50% to about 97% by weight.

41. The method of any one of claims 31-40, wherein the transdermal delivery device comprises a drug-in-adhesive layer, and the drug-in-adhesive layer further comprises a gallate antioxidant such as propyl gallate.

42. The method of any one of claims 31-41, wherein the transdermal delivery device comprises a drug-in-adhesive layer, and the drug-in-adhesive layer further comprises a crystallization inhibitor in an amount effective to prevent formation of drug crystals after shelf storage for two weeks at ambient temperature.

43. The method of any one of claims 31-42, wherein the transdermal delivery device comprises a drug-in-adhesive layer, and the drug-in-adhesive layer further comprises a crystallization inhibitor selected from a polyvinylpyrrolidone polymer (e.g., Kollidon K30 or K90F (manufactured by BASF), Plasdone K20/32 or Plasdone K90 (manufactured by Ashland Chemical)), a cross-linked polyvinylpyrrolidone polymer (e.g., Kollidon CL), a polyvinylpyrrolidone copolymer (e.g., Plasdone S-630Copovidone (Asland)), a cellulose based polymer (e.g., hydroxylpropyl methyl cellulose, ethyl cellulose, hydroxypropyl cellulose), a polycarboxylic acid polymer (e.g., Cabopol (manufactured by Lubrizol)), a polymethacrylate (e.g., Plastoid B, Eudragit E100, Eudragit L100-55 (manufactured by Evonik)), a polyethylene glycol, polyvinyl acetate and polyvinylcaprolactame-based graft copolymer (PVAc-PVCap-PEG) (e.g., Soluplus (BASF), and combinations thereof.

44. The method of any one of claims 31-43, wherein the sole active ingredient in the transdermal delivery device, e.g., in the drug-in-adhesive layer, is, as applicable, a substantially pure R,R-isomer of tetrabenazine or a substantially pure deuterated R,R-isomer of tetrabenazine.

45. The method of any one of claims 31-44, wherein the hyperkinetic movement disorder is selected from Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.

46. The method of any one of claims 31-45, wherein the administering is conducted without regard to the fed status of the subject.

47. The method of any one of claims 31-46, wherein the subject is an extensive metabolizer.

48. A method of identifying a pharmaceutical composition for treating a hyperkinetic movement disorder, the method comprising administering a test pharmaceutical composition to a subject that bypasses first-pass metabolism to deliver R,R-tetrabenazine to the subject continuously or substantially continuously, and identifying a pharmaceutical composition that provides a therapeutically effective plasma concentration of R,R-tetrabenazine, R,R,R-dihydrotetrabenazine (HTBZ) and S,R,R-HTBZ, wherein the ratio of the maximum plasma concentration of R,R-tetrabenazine to the combined maximum plasma concentration of R,R,R-HTBZ and S,R,R-HTBZ ranges from about 1:1 to about 1:5 or the ratio of the steady state plasma concentration of R,R-tetrabenazine to the combined steady state plasma concentration of R,R,R-HTBZ and S,R,R-HTBZ ranges from about 1:1 to about 1:5, preferably, the ratio of maximum plasma concentration or steady state plasma concentration of R,R,R-HTBZ to S,R,R-HTBZ ranges from about 1:5 to about 1:30 (e.g., about 1:10 to about 1:20), preferably, administering the identified pharmaceutical composition provides no detectable S,S-tetrabenazine, R,S,S-HTBZ, or S,S,S-HTBZ.

49. A method of identifying a pharmaceutical composition for treating a hyperkinetic movement disorder, the method comprising administering a test pharmaceutical composition to a subject that bypasses first-pass metabolism, and identifying a pharmaceutical composition that when administered to provide a dose of about 2 mg/day for 4 days, provides a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 300 pg/ml to about 700 pg/ml;
d) a mean Cₘₐₓ of R,R,R-HTBZ of about 60 pg/ml to about 200 pg/ml;
e) a mean Cₘₐₓ of S,R,R-HTBZ of about 1000 pg/ml to about 3000 pg/ml;
f) a mean AUC₀₋₉₆ of R,R-tetrabenazine of about 20 ng*h/ml to about 50 ng*h/ml;
g) a mean AUC₀₋₉₆ of R,R,R-HTBZ of about 4 ng*h/ml to about 12 ng*h/ml; and/or
h) a mean AUC₀₋₉₆ of S,R,R-HTBZ of about 70 ng*h/ml to about 200 ng*h/ml.

50. A method of identifying a pharmaceutical composition for treating a hyperkinetic movement disorder, the method comprising administering a test pharmaceutical composition to a subject that bypasses first-pass metabolism, and identifying a pharmaceutical composition that when administered to provide a dose of about 4-6 mg/day for at least one day, for example, two days, three days, four days, or a week, provides a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 600 pg/ml to about 2100 pg/ml;
d) a mean Cₘₐₓ of R,R,R-HTBZ of about 120 pg/ml to about 600 pg/ml;
e) a mean Cₘₐₓ of S,R,R-HTBZ of about 2000 pg/ml to about 9000 pg/ml;
f) a mean AUC₀₋₉₆ of R,R-tetrabenazine of about 40 ng*h/ml to about 150 ng*h/ml;
g) a mean AUC₀₋₉₆ of R,R,R-HTBZ of about 8 ng*h/ml to about 36 ng*h/ml; and/or
h) a mean AUC₀₋₉₆ of S,R,R-HTBZ of about 140 ng*h/ml to about 600 ng*h/ml.

51. A method of identifying a pharmaceutical composition for treating a hyperkinetic movement disorder, the method comprising administering a test pharmaceutical composition to a subject that bypasses first-pass metabolism, and identifying a pharmaceutical composition that when administered to provide a dose of about 1-10 mg/day for at least one day, for example, two days, three days, four days, or a week, provides a pharmacokinetic profile in the subject characterized by
a) a mean Tₘₐₓ of R,R-tetrabenazine of about 10 hours to about 24 hours, e.g., about 20 hours;
b) a mean Tₘₐₓ of R,R,R-HTBZ and mean Tₘₐₓ of S,R,R-HTBZ later than the mean Tₘₐₓ of R,R-tetrabenazine;
c) a mean Cₘₐₓ of R,R-tetrabenazine of about 150 pg/ml to about 3500 pg/ml;
d) a mean Cₘₐₓ of R,R,R-HTBZ of about 30 pg/ml to about 1000 pg/ml;
e) a mean Cₘₐₓ of S,R,R-HTBZ of about 500 pg/ml to about 15 ng/ml;
f) a mean AUC₀₋₉₆ of R,R-tetrabenazine of about 10 ng*h/ml to about 250 ng*h/ml;
g) a mean AUC₀₋₉₆ of R,R,R-HTBZ of about 2 ng*h/ml to about 60 ng*h/ml; and/or
h) a mean AUC₀₋₉₆ of S,R,R-HTBZ of about 35 ng*h/ml to about 1000 ng*h/ml.

52. A method of identifying a pharmaceutical composition for treating a hyperkinetic movement disorder, the method comprising administering a test pharmaceutical composition to a subject that bypasses first-pass metabolism, and identifying a pharmaceutical composition that provides a pharmacokinetic profile in the subject characterized by that (1) during a first time period, a maximum plasma concentration of R,R-tetrabenazine of about 150 pg/ml to about 3500 pg/ml is reached for the first period of time at a first time point, wherein the first period of time is from the time of initial administration to about 24 hours thereafter; and optionally (2) after the first time point, the plasma concentration of R,R-tetrabenazine remains substantially constant for a sustained period of about 24 hours, about 48 hours, about 72 hours, about 96 hours, or more, and preferably, the average terminal half-life of R,R-tetrabenazine is about 8.5 hours±40% CV.

53. The pharmaceutical composition identified by any of the methods of claims 48-52.

54. A method of treating a hyperkinetic movement disorder in a subject in need thereof, the method comprising administering to the subject a therapeutically effective amount of the pharmaceutical composition of claim 53.

55. The method of claim 54, wherein the hyperkinetic movement disorder is selected from Huntington's disease, Wilson's disease, Tourette syndrome, restless leg syndrome, tardive dyskinesia, tic, dyskinetic cerebral palsy/cerebral palsy, other dystonia and dyskinesia disorder, and combinations thereof.
